SEQUENCE LISTING (414 sequences)

SEQ ID NO: 1 - DNA, 432 bp, Homo sapiens
gaactctgcaaacctcctggatttagcaggagacaacatgagggtaatcaccccggcacctggacccattagattaagtcaatttactgaggctcctgaggatgatgctcaggactcagaccttagttatagattaaaagaagttaaggccgggcgcggtggctcacgcctgtaatcccagcactttgggaggccaagatgggcggatcacgaggtcaggagatcgagaccatcttggctaacaccgcgaaaccccgtctctactaaaaatacaaaaaaatcagccgggcgtagtggcgggcgcctatagtgccagctacccggaggctgaggcaggagagtggcgtgaacccgggaggcggcgcttgcagtgagctgagattgcgccactgcactccagcctgggcgacagagcgagactccgcctcaaaa

SEQ ID NO: 2 - DNA, 491 bp, Homo sapiens
ctgtgcaagagaatattcccgctctccggagaagctcttccttcctttgcactgaaagctgtaactctaagtatcagtgtgaaacgggagaaaacagtaaaggcaacgtccaggatagagtgaagcgacccatgaacgcattcatcgtgtggtctcgcgatcagaggcgcaagatggctctagagaatcccagaatgcgaaactcagagatcagcaagcagctgggataccagtggaaaatgcttactgaagccgaaaaatggccattcttccaggaggcacagaaattacaggccatgcacagagagaaatacccgaattataagtatcgacctcgtcggaaggcgaagatgctgccgaagaattgcagtttgcttcccgcagatcccgcttcggtactctgcagcgaagtgcaactggacaacaggttgtacagggatgactgtacgaaagccacacactcaagaatggagcaccagctaggccactta

SEQ ID NO: 3 - DNA, 251 bp, Homo sapiens
atcacaaagaactatgtcggaattcttctgtgtagtttttatgtgaagatatttccttttccacaatagacgtcaaagtgatccagatatccacttgcagattccacaaaaagagtgtttcaaaagtgcacaaccaaaagaaaggttcaactaggtgagatgaatgcacacatcagaaggaagtttctcagaatgcttctgcatagcttttaagggaagatacttccttttccaacataggcctcaaagca

SEQ ID NO: 4 - DNA, 422 bp, Homo sapiens
gactggagtggaacggtctcgaatggaatggaatggattggaatggaaaggaatagaatggaatggaatcatatggaatggaatggaacagaatgagtcaaaacggaatagaatcaagtggaatgcaatcgaatggaatggaatacaatggactcgaatggaatggattctaatggaatagaatataatggaatggcatggaatggaatgaaatagccagctccctgtgcaggtgaaaatccatgtataacttttgactccccaaaagcttagttacttatcacctactgttgactagaagcctgactcgtaacatagtcaagtaatacacattttatatgttatgtgtattatatactgtactctaacaaaaagtaagctgaagaaaggaatatgttattaagaaaatcggagggaagtga

SEQ ID NO: 5 - DNA, 487 bp, Homo sapiens
gagattacaggcatgcaccaccatgcccagactcttactggtctttttaatatgtaagacagtggtaccttttttccttttaggttatgaaatggtctctgcaagatgttttgtccttgaattggaaatttttaaggctgatatatgctggtacttcatcttctatatgtggactataatttcttcccttaggataactacataaagagacaaaaaaaagaaaaaagagcaaagatctgtgctgtgtcaagtatgacagccatcactcatggctctccagtaggagggaacgacagccagggccaggttcttgatggccagtctcagcatctcttccaacagaaccaggtaggagtaagactgtgttgttttgagtactgtgaaatacaaaattgtgaaacatgtcctgaatgatttgtaaagtaatcataaaatatgtggttattttaagttacacgtgaaaaaagtgactgtgggctaagtgtgg

SEQ ID NO: 6 - DNA, 350 bp, Homo sapiens
ttacccctttcacccactgaaaataagtttgtagatgtaatctgcaaattataaagagaaagtgatgaatttttaaaaacaattgaagttttgggttatgaatttaaaattatatttatgttttaggaaaatggaaggtatgggcgccgcaaacaatatccaatatccttggttttagccccaacaagagaattggctgtacagatctatgaggaagccagaaaagtaaaatattcattttagtgattattgcttttcttatttgtcaaatgatgttgttatagtcactgacatgttctttgcttaaagttttcctaccgatctagagttcgtccttgtgtagtttatgg

SEQ ID NO: 7 - DNA, 335 bp, Homo sapiens
aggaatcatttgaggcccttgtgtacaaaaatggttaccagaagactttaaaagagtgggaacaaatgattgtggttttcactatcttcatcctttactgtgccatagttaccaggtccttacactttcttctaggattggcagcctccttttgcagtagaagttgacaatttcagatttactcctcgcgtccaaaggctaaatgaactggaggtaagattgggaggcacactttttttaaaggaatctgatctttaatcttgccgttgtagtttcataataatgtagaacttaagttttgaaatctaatgtattgaatttgaacccgaactcca

SEQ ID NO: 8 - DNA, 672 bp, Homo sapiens
gaggaaagcagatatttccaaatagtacttaactattcatgctttaatgatagcagtaaaaatgtttaaatgtagtcccacatattattttaccaaccctgcagggacctctcatggtgcaccacctgcaagagggcctcggatgtcttatggtggaagcacctgccacgcatatagtaatacacgagatagatatggcagaagttgggagagttactcgagctgtggtgattttcattattgtgatcgtgagcatgtttgcagaaaagaccaaaggaatccgccttctctgggtagggtgctccctgatcctcgtgaagcatatggtagctcaagttatgtggcatctatagtagatggtggggagagtcgatctgaaaaaggagactcgagcagatattaaagcaagcattgaaagtaatagttattgcataccaatccttgtttgcacatcaaaaattgaaatgttatttctgcattgttacctgcatattactgaaagaaacatgttggttttgtggagagaggtagatactaacttcctccatgaattttttgaggtattcaaaggaaaaggaattgttttcaaagtaatttcatacttgttgatgctatttgaaaagtgtttagatgtaatatctaccttaaaattttcacaataaaatttgacat

SEQ ID NO: 9 - DNA, 393 bp, Homo sapiens
gttggcaacttgcactgctcactgcagcctcctctgcagagcttcaagtgatcctccgacttctcagctacttgagtagctacaggctctcgctatcagagctagtaggcaatttttttgtgtgtttctttaatggacatgggtttccccatggtacccaagctggtctcaaacttgggttaagttaagttaagatctacctctccctccaaatcagagataaccaggcaactttaagaactaaagttgactgtggagaaaatacttagacagccctcttggaacatcagcctggtaattggttagagctcctagcttaagaggtgagaaagacaggtcacttctgggcaagctctggaacctcacgatacttggggactttgagaggaaagg

SEQ ID NO: 10 - DNA, 453 bp, Homo sapiens
tcatttgtgtccttctcttggatttactaatttactagttaagctgtgttatgttcactggacatttaagattttataaatatctatatggcaatagagtcatttctgaggtagtaagaatgtattctcctttcaacagaacctaattggaacctggttttattacaaagccttgtctgaaatatcaaatacatttaagaatgatttgcagaaaatcagagataaccaggcaactttaagaactaaagttgactgtggagaaaatacttagacagccctcttggaacatcagcctggtaattggttagagctcctagcttaagaggtgagaaagacaggtcacttctgggcaagctctggaacctcacgatacttggggactttgagaggaaaggtattcaaccaagtgtataggttctgaaagggaagcctggtggcaagttcctggcttag

SEQ ID NO: 11 - DNA, 601 bp, Homo sapiens
ctatcagacactattttggcaattttatgtacctaaacatgttaaataatcatgcttaccattttttccagacgttctaccctttcgagattagttaatatgtttacacacagagttttctttataggattataatttacaatgttttcacaattttcttaaacagtcgactttattttatttaactttaagacaacttttttattcttaagcaaaatacatagttatgccttataatttttaactaaaaccactttttaccatttttatacacttttatgcaaatccatgtttagcagttttaattacctgttataacggtaatttttagcaatttttaactttaatgtaaagcctattacgtgttttttattttgttttgttttgttttctttttgagacagagtcttgctctctcatcaggctagagtgtggtaactggatctcggctcactgcaacctccacttcttggatacaagcgattctgctgcctcagcctcctgagtagcttggattacagacgcctgccaccacacccagctaattattgtatttttagtagggaggaggtttcaccatgttttccaggattgtcttgat

SEQ ID NO: 12 - DNA, 601 bp, Homo sapiens
tttttaaaaatgaaagattattcttgttttcactgtgaagcacaataacaataaattttccccattggtacaagtgaatgatttacatggtaaattgatgtgcttaactactaccatttctggaagccggatttgatataaacttattttgggctgggcgcggtggctcacgcctgtaatctcagcagtttgggaggccgaggcaggtggatcacgaggtcaggagatggagaccatgctggctaacacagtgaaaccccgtctctactaaatacacaaaaaaattagccgggtgtagtggtgggcgcctgtgttcccagctactccggaggctgaggcaggagaatggcatgaaccaagggagcggagcttgcagtgagctgagatcgagccactgcactccagcctaggcgacagagccagactccgtcttaaaaaaaaaaacaacaaaaaacttattttgataaacatggcttatgatacttgataataaaattaataaagatgttgtttttataaacatcaaatgtgaatagctgttgtcatggtttaaaatgtcaaaggacagcctttgaaaattaagatactgataacagacatg

SEQ ID NO: 13 - DNA, 531 bp, Homo sapiens
tgatatcaggtgatgcgtcctcctcggcctcccaaagtgctgggattagaggtgtgagccactgctcccagcctcttttagcatttttgcatttctttggaaataaactgatatgttcattaaaccatcaaaagaaaaaccaaaacacacccttattaagagtgagtgaaagaaagagttgtctttacattactgaaaacttctgtgtttcagaaatctgtggaccgaagcatacaaatggtggtatcttgtctgtttaatccagagaagagactgataaattccgttgttactcaagatgactgcttcaaggtatgaaaggaatggcatgcataattaaaaagcacacttgttccctctcaagttagctgttttccttgtggcacatgtattttgggctttcttagaggaattttttttcttttttttttgttttgagacggagtctcctctgtcgcccaggctggagtgcagtgagtggccccatctaggctcactgcaagctccacctcccaggtttacttaacgcca

SEQ ID NO: 14 - DNA, 147 bp, Homo sapiens
cccagtcttcatcagctgcagctagccaaggctgggtgttaccagaaggcaaaatcgtgctaaacactgtttttgttggtggaattgatgctaggatggatgaactgagattggaagctggtttggtagatacggttcagtgaattg

SEQ ID NO: 15 - DNA, 527 bp, Homo sapiens
taaaaggcagaactgccaggtctgtgtcttattttctttgtcattctaatttatctttttttttttttttttttttttttttttttttttttttgagacggagtctcactctgtcgcccaggctggagtgcagtggcgggatctcggctcactgcaagctccgcctcccgggttcacgccattctcctgcctcagcctcccaagtagctgggactacaggcgcccgccgctactcccggctaattttttgtatttttagtagagacggggtttcaccgtttttagccgggatggtctcgatctcctgacctcgtgatccgcccgcctcggcctcccaaagtgctgggattacaggcgtgagccaccgcacctggccaagtgtcttcttttgagaagtgtctgttcatatacttcacccactttttgatggggttgtttgtttttttcttgtaatttttgtttgagttcattgtagattctggatattagccctttgtcagatgagtacgttgcagaacttttctccc

SEQ ID NO: 16 - DNA, 755 bp, Homo sapiens
tgggcgaggactttatgactaaaacaccaaaagcaatggcaacaaaagccaaaattgacaaatgggatctaattaaactaaagagcttccgcacagcaaaataaaccactgtcagagtgaataggcaacctacagaatgggagaaaagttctgcaacgtactcatctgacaaagggctaatatccagaatctacaatgaactcaaacaaaaattacaagaaaaaaacaaacaaccccatcaaaaagtgggtgaagtatatgaacagacacttctcaaaagaagacacttggccaggtgcggtggctcacgcctgtaatcccagcactttgggaggccgaggcgggcggatcacgaggtcaggagatcgagaccatcccggctaaaaacggtgaaaccccgtctctactaaaaatacaaaaaattagccgggagtagcggcgggcgcctgtagtcccagctacttgggaggctgaggcaggagaatggcgtgaacccgggaggcggagcttgcagtgagccgagatcccgccactgcactccagcctgggcgacagagtgagactccgtctcaaaaaaaaaaaaaaaaaaaaaaaaaaaaaaaaaaaaaaagataaattagaatgacaaagaaaataagacacagacctggcagttctgccttttaagggccagcctcagcctagtcaccgtgaatcacaatttcaggttctgcgtcagcgtgtcccaccttggaaaatagtggaactgggacccc

SEQ ID NO: 17 - DNA, 461 bp, Homo sapiens
aggaatggaatgggatggagtggaatggagtggagtggagtgcagtggagtggagaggagtggaatagagtggaatggaataggatgtaatggaatgtagaggagtggagtggagtcggagtcggagtgtgatggaatgtaatggaaaggaatggaatgcaattgaatgcaaagaaatggaaagttgacatgtaatgtgacctgagattgtgccactgcactccagcctctgtgacagagtgagatcctttggaaagaaaggaacggaatggtatggaatggagtggagtagagtggagtggaatggaatggaatggaatgcaatggaaagcaatggaatgcaatggaatagaatgcagtgaaaaggaaagttgacatgtaatgtgagcagagattgtgccactgcactccagcctgggtgacacagtgatatcctgtcaaaagaaaggaatggaatgcaa

SEQ ID NO: 18 - DNA, 645 bp, Homo sapiens
gagctgcaacataacaggcactataaatgaatgaatggataaagacagcactgaagaacttgaaattatagtttacacatgtaaacagtaacataaatctgaagaaataaagtttttttttgttgttttttcgagatggagtctcgctctgtcaccaggctggagtgcagtggtgcaatctggcctcactgcaacctccgcctcccgggttcaagcgattcccctgcctcagcctcagcgtcccaagtagctaggactccaggcacgtgccactacgcctggttatttttctttctttctttttattgtattttagtagagatgggtttcactatattggccaagacggtctcaatctcctgaccttgtgatctgcctgcctcatcctcccaaagtgctgggattacaggcgtgagccaccgggcctggccaagttgttgttgttgttgttgttgttgttgtttttattatactctaagttttaggttacatgtgcacattgtgcaggttagttacatatgtatacatgtgacatgctggtgcgctgcatccactaactcatcatctagcattaggtatatctcccaatgctatccctccccctccacccaccccaccacagtccccagagtgtgatgttcccct

SEQ ID NO: 19 - DNA, 998 bp, Homo sapiens
agatgtcccagatgcgtaggacagagtcatctagtcatcttccagggtttaatcctggactactggaaacaactagaagtgagaaaagcgtctgtgctttgcagtgaccattgtcctgtcagacaaccaataaatgctatctttcctctttcaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagggtggccactgcagcacctgccactgccgtcacctctccctcctcagctccagggcagtgtccctccctggcttgccttctacctttgtgctaaatcctagcagacactgccttgaagcctcctgaattaactatcatcagtcctgcatttctttggtggggggccaggagggtgcatccaatttaacttaactcttacctattttctgcagctgctcccagattactccttccttccaatataactaggcaaaaagaagtgaggagaaacctgcatgaaagcattcttccctgaaaagttaggcctctcagagtcaccacttcctctgttgtagaaaaactatgtgatgaaactttgaaaaagatatttatgatgttaacatttcaggttaagcctcatacgtttaaaataaaactctcagttgtttattatcctgatcaagcatggaacaaagcatgtttcaggatcagatcaatacaatcttggagtcaaaaggcaaatcatttggacaatctgcaaaatggagagaatacaataactactacagtaaagtctgtttctgcttccttacacatagatataattatgttatttagtcattatgaggggcacattcttatctccaaaactagcattcttaaactgagaattatagatggggttcaagaatccctaagtcccctgaa

SEQ ID NO: 20 - DNA, 909 bp, Homo sapiens
gtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccacca

SEQ ID NO: 21 - DNA, 20 bp, Artificial Sequence
  probe for STS
gaactctgcaaacctcctgg

SEQ ID NO: 22 - DNA, 18 bp, Artificial Sequence
  probe for STS
ttttgaggcggagtctcg

SEQ ID NO: 23 - DNA, 20 bp, Artificial Sequence
  probe for STS
gaatattcccgctctccgga

SEQ ID NO: 24 - DNA, 20 bp, Artificial Sequence
  probe for STS
gctggtgctccattcttgag

SEQ ID NO: 25 - DNA, 22 bp, Artificial Sequence
  probe for STS
tccttttccacaatagacgtca

SEQ ID NO: 26 - DNA, 26 bp, Artificial Sequence
  probe for STS
ggaagtatcttcccttaaaagctatg

SEQ ID NO: 27 - DNA, 20 bp, Artificial Sequence
  probe for STS
gactggagtggaacggtctc

SEQ ID NO: 28 - DNA, 20 bp, Artificial Sequence
  probe for STS
tcacttccctccgattttct

SEQ ID NO: 29 - DNA, 20 bp, Artificial Sequence
  probe for STS
gagattacaggcatgcacca

SEQ ID NO: 30 - DNA, 20 bp, Artificial Sequence
  probe for STS
ccacacttagcccacagtca

SEQ ID NO: 31 - DNA, 20 bp, Artificial Sequence
  probe for STS
ttacccctttcacccactga

SEQ ID NO: 32 - DNA, 24 bp, Artificial Sequence
  probe for STS
ccataaactacacaaggacgaact

SEQ ID NO: 33 - DNA, 20 bp, Artificial Sequence
  probe for STS
aggaatcatttgaggccctt

SEQ ID NO: 34 - DNA, 20 bp, Artificial Sequence
  probe for STS
tggagttcgggttcaaattc

SEQ ID NO: 35 - DNA, 20 bp, Artificial Sequence
  probe for STS
gcacctgccacgcatatagt

SEQ ID NO: 36 - DNA, 20 bp, Artificial Sequence
  probe for STS
gcaaacatgctcacgatcac

SEQ ID NO: 37 - DNA, 19 bp, Artificial Sequence
  probe for STS
gttggcaacttgcactgct

SEQ ID NO: 38 - DNA, 20 bp, Artificial Sequence
  probe for STS
cctttcctctcaaagtcccc

SEQ ID NO: 39 - DNA, 22 bp, Artificial Sequence
  probe for STS
tcatttgtgtccttctcttgga

SEQ ID NO: 40 - DNA, 20 bp, Artificial Sequence
  probe for STS
ctaagccaggaacttgccac

SEQ ID NO: 41 - DNA, 20 bp, Artificial Sequence
  probe for STS
ccagacgttctaccctttcg

SEQ ID NO: 42 - DNA, 20 bp, Artificial Sequence
  probe for STS
gagccgagatccagttacca

SEQ ID NO: 43 - DNA, 20 bp, Artificial Sequence
  probe for STS
actaccatttctggaagccg

SEQ ID NO: 44 - DNA, 20 bp, Artificial Sequence
  probe for STS
ctcccttggttcatgccatt

SEQ ID NO: 45 - DNA, 20 bp, Artificial Sequence
  probe for STS
tgatatcaggtgatgcgtcc

SEQ ID NO: 46 - DNA, 20 bp, Artificial Sequence
  probe for STS
tggcgttaagtaaacctggg

SEQ ID NO: 47 - DNA, 20 bp, Artificial Sequence
  probe for STS
gggtgttaccagaaggcaaa

SEQ ID NO: 48 - DNA, 22 bp, Artificial Sequence
  probe for STS
gaaccgtatctaccaaagcagc

SEQ ID NO: 49 - DNA, 20 bp, Artificial Sequence
  probe for STS
taaaaggcagaactgccagg

SEQ ID NO: 50 - DNA, 20 bp, Artificial Sequence
  probe for STS
gggagaaaagttctgcaacg

SEQ ID NO: 51 - DNA, 18 bp, Artificial Sequence
  probe for STS
tgggcgaggactttatga

SEQ ID NO: 52 - DNA, 20 bp, Artificial Sequence
  probe for STS
ggggtcccagttccactatt

SEQ ID NO: 53 - DNA, 20 bp, Artificial Sequence
  probe for STS
agtcggagtcggagtgtgat

SEQ ID NO: 54 - DNA, 20 bp, Artificial Sequence
  probe for STS
attccattgctttccattgc

SEQ ID NO: 55 - DNA, 20 bp, Artificial Sequence
  probe for STS
gagctgcaacataacaggca

SEQ ID NO: 56 - DNA, 20 bp, Artificial Sequence
  probe for STS
aggggaacatcacactctgg

SEQ ID NO: 57 - DNA, 20 bp, Artificial Sequence
  probe for STS
agatgtcccagatgcgtagg

SEQ ID NO: 58 - DNA, 22 bp, Artificial Sequence
  probe for STS
ttcaggggacttagggattctt

SEQ ID NO: 59 - DNA, 22 bp, Artificial Sequence
  probe for STS
gtctatgggacccttgatgttt

SEQ ID NO: 60 - DNA, 19 bp, Artificial Sequence
  probe for STS
tggtggggtgaattctttg

SEQ ID NO: 61 - DNA, 491 bp, Homo sapiens; identical to SEQ ID NO: 2

SEQ ID NO: 62 - DNA, 401 bp, Homo sapiens
cccatcttgcacctctgaagcttaaggggacagtatttcaacttcggccttttttataggagaatgagccaagctctggttactttacttactgtgtctctttccacacctcctaggcaggttaaaagcagtgataagagagaagctggcaaacctagttagtcctgagagcccaaagatacgggagagaggaaatgcagcagctgattaaaaaacaaaaaagtggctcctcgagttcatttattgtacagtttctggagtatttctgtcatcttttcagggagtttttctgtcatctggcactctgtttgtccttgtcattttcatatttacccagaatttgtcctgagacttcagaggactgtactcagtttaaatgtggagtttttaatatcttttaa

SEQ ID NO: 63 - DNA, 793 bp, Homo sapiens
tggcagactggctaaacagaaaccaaagaaaaagagaagacctgattccaggcagtaccaaacaggtgagttccacaggggtgttatgatggagttttagctagtaggccacatgtatttttatgtgttgaatttgaaagaaaaaatttcaaaattcagtgatattcatgaatggttttcttggataagaagaaacagttgtgcatcaaccattcaggaaaaagaattaaaatcctctggtgattttagtgaaaaggaaaataaatttctaaaatgttacctaactttaagtgaacgaaattacatggatctacttatactagcataaagcaggtataatttaccgagaagtggaagaagtacctaggttatttgtaggactgattactatcctatgttgttttgaatgctgctttgcatattaaaatttatttataggtgcagttaagctttactgtttgcatatatttggcttggagtcagtcaccaaagcagaaatgctggacttgatttttatgagttatttgctacacatttctaaattcatgttcttttgtcactgcttgattgatttttttttttaactggagggggtgagattggttcatacttaaccaaaccagttctctcgattaggacattattataactcttaacattgaaaagcagtaaaaggaatgttaataatttaaaagtatttgcccactaatgttcagaacacaagctttaaaaaattcatgaggagaccagaagtttgattaagcactcatactgctttcttttcctttcttag

SEQ ID NO: 64 - DNA, 381 bp, Homo sapiens
gaccaatttgtctttgttgcggatttctaactggtttatcaatgctcgcagacgcattctcccggatatgcttcaacagcgtagaaacgaccccatcattggccacaaaacgggcaaagatgcccatgccacccacctgcagagcaccgaggcgtctgtgccggccaagtcagggccagtggtccagacaatgtacaaagcctgcccctgtggcccttgccaaagggccagatgtcaagagagaagcaaccagatccggagtcggcccctagccagaagctcaccggaatagcccagccaaagaaaaaggtcaagatttctatcacttccccgtcttctccagaacttgtgtctccagaggagtacgccgacttcagcagc

SEQ ID NO: 65 - DNA, 385 bp, Homo sapiens
gggtcctagataggctccaaggaaaccaaattacttgaagaagcctgagattattcttaaagaaatatagatttccttgtatgtttacaatgaatgagtcttatttttacatgcatggaagtatcacagtcactggacactgtataatgaaaacttacaagttctcattttgaacaagcttcctttttccttagttgacaaaattatccaggtttcttttagcatgttcacaatttcagattttatacaattacaaactgaactttccttataaactgattgttatagctaatttttcctaataaaattcaaattaagacagcacctgtgaattctatttagagttctattgatactcaatgtagccaatcactgccaacatgaa

SEQ ID NO: 66 - DNA, 260 bp, Homo sapiens
cccagtgaagaatcgtccatttccagaatcaatgagaagtaaagctgaaaatcattcagttcagtctgtggcacttgattccacggctgtcaaccccaccggcagtcatcccaccaaccccatgagattgggctccctgaatgtgcgtcctggtcatccttgccccaaaccacaaaggactgtttagattgatggatttccttaagctgttgccccatcagacttgtgtgtgcttttagggcccagtgcatcttgttagc

SEQ ID NO: 67 - DNA, 420 bp, Homo sapiens
tagaactcacattctcaggctatcaatgttgacaggattgctttagtgagtctatatttcctaccgcatcagtgaatttctgcatgggatgaaagtaaattaaatcaaatggattctaatatatctttctcttaaggtgctcacccctttgaagtggtaccagagcatgataagaccaccagtatgtagacattttgttctttattccctgaaaatattaggcatgcatttaaattcccattttaagaaaataccatgtgtactccacatacagacactaatgggaaatttagtttgtaaaaaatcatgtctgtgtacacagttacaaatttttgcaaaggaaagataaatacaatattcctatggccataatggcaaagacaacactgctgcttctctggttggagtcacgtgagccaa

SEQ ID NO: 68 - DNA, 375 bp, Homo sapiens
ggtgtgctaacattgcatggcaccaactattgcctgaagaaccctctggacttgggatcctgagagaggctctctgagcttctttggtgctcccactcctaagctgtttttctgagtttgtatctctttctggccctcagagtgatggaacactattggctatgggttcatatgatggtttcgcaagaatatggacagaaaatggtaagtcctgcacccctcctgcatgggtcaggtaagaggagctgccatacataacccttgattgaacagaccatgacaacagaatcaacatgtttgtttttactaacaggtaacctggccagcaccttaggccaacataaaggccccatcttcgctctgaaatggaacaaa

SEQ ID NO: 69 - DNA, 362 bp, Homo sapiens
ggctgtgcaggttgtttcataggcaaacgtgggccatgctgctttgttgtttttcactttttaataatcgccattccgactggcatgagataacaactcattatggttttcatgtacatttcattacccaggtattgagcctggcatccattagctattcttcctgatgctctccttccccctgcgcccccttacaacaggccccagagtgtgctcttccccacaatgtgtccatgtgttctcatcgttcagctcccaattataagtgagaacatgcagtgtttggttttctcttcctgtgttagtttcctgaggataacagcttccagcttcatccacgttcctgcaaaggacataatctc

SEQ ID NO: 70 - DNA, 303 bp, Homo sapiens
acctgtctgggaaacacctcatctctacaaaatacaaaaaagttaactgggcatggtggagcatgcctgtagtctcagctactcaggaggctgagatgagaggatcacttgagcctgaggaggtccaggccacagtgatctgagatcacaccattgccctccagcctgggtgacagagtgagaccctgttccaaaaaaaaaaaaaaacccaaaaaacctcaatggctttttgcttttgaaggtggtatgaagagaacgtccactttaaggctttaaaagacagtgaagctggttgtagggctc

SEQ ID NO: 71 - DNA, 493 bp, Homo sapiens
tttttctaaccttgcctgcggtttgcaccatttattacatttttccaacaaccaaaggttggctttgtatgttttactaattttctctacatcattatcccctcactttagtttttcagaattgattctgttgtttctgttctaattctttctttaaatatctagtacattaattttcaagttgtagaaacatttgtctatgaactcctattgcaatatcacttttcctgctacccacaaatttaatctgtaatatttgcagtaccattaatttctattatgaatttatattatgataatccatgagttgctgagaaatagctgttataattttgttgttcaattcccatttaattttattttaacttgtgctaactcagttgaaaattctttactaattttttaaatctcatatcaagacttttattcacatcaattgttctataaatgctccctccttgaagaacactctcttgtaggctgcttctctgca

SEQ ID NO: 72 - DNA, 251 bp, Homo sapiens; identical to SEQ ID NO: 3

SEQ ID NO: 73 - DNA, 422 bp, Homo sapiens; identical to SEQ ID NO: 4

SEQ ID NO: 74 - DNA, 487 bp, Homo sapiens; identical to SEQ ID NO: 5

SEQ ID NO: 75 - DNA, 463 bp, Homo sapiens
aaggcaggtctgatgcatgtttacttttacttccatttccacttctttcactaagagaaatattttgttttcctgattattcactttaatgttctgccagcacttttaattatttatttcttttctgcaggcttcaagatatatgcctgatatttgtgtaattagggctatacagaaaattatctgggcatcagcatgtggggcattaggactagtttttagcccaaatgaagaaataactaaaatttatcagatggtaagaattattacagaaatagatttttaagaaaatgttgcttcattgtacatgtgattaaattttcatcatttctgtcaccttatagaccaagtcgcttgtatctaatggtttaaaatttattgctacctataaataaaatgagaatatattgttttattttggaaataaaatactctagaagcctgctatgaggcagctttcttt

SEQ ID NO: 76 - DNA, 350 bp, Homo sapiens; identical to SEQ ID NO: 6

SEQ ID NO: 77 - DNA, 292 bp, Homo sapiens
ttgcacccgtagtcaaatgtatgttccatagttttagcatttgcgtatgtctgacattttatattatccctactttttgttactttattacattagactgtattgtaactgatcttttcccatgatccattgcagttcaatttcatattgcccatttgtatgaaacccaggtaagtgttttaacttaagaacaagaatgaaacagtcacctatgctttagatgaaatgaaatgtacttgaaaatatcaggctttaaatgaaataaatatattagacgatttgagttgtgggt

SEQ ID NO: 78 - DNA, 574 bp, Homo sapiens
ctcttccaagccagcctttaattttaaacgctgtaattaacagttcacaggggtcaaattcctttattccggaacattccactttgagagggatctgtcctctttggtcccctgcgttttcaaatatttgaggaaaggtgtcgcctctttttctgtggaaagaggaagctcatgagcgcgaaacagcaggggacggagggcgagaagggctttctcaggttgcgggtcggagggcagaagcacagttcccagtacagagacccggacaggtggctgtttctcacgctcactttggattgctccctacggcttcctccgcagccatgtctgacaaacctggtatggctgagatcgagaaattcgataagtcgaaactgaagaagacagaaacgcaagagaagaatccattgtcttccaaagaaagtaagctccgatcctcccccatctttagaaaggctggaatgcgagcgggcggtgggagggcgggagactgggagctgccacgggaagaattcgggaggcaggggagggcgctcaaggagcagatagttggtgaatgtggcttgcaaaggtt

SEQ ID NO: 79 - DNA, 514 bp, Homo sapiens
  misc_feature 8, 125, 169, 218, 235, 240, 258, 296, 309, 349, 376,378, 403, 405, 435, 438, 442, 465, 503: n = A,T,C or G
tgtaccgnagtacaaacttatccatagatttaatacaagcagtgccccataacactttcataagtctctctaccgtagtcctaacattttataccattcactgaaacataccagccaggttagancataaaatgtgatgtaaaacttgtaaaacacccagcagagtggntgcttcttagtaatcttccaggcagagcgagctgctgtattaccattgngtatattattccaacangtttngtgtcctgattgcatctngcagggcaggtgcttctgtcccccactggcatgcatgnggggttgatattnaggaagatggtgtaatccaccctacctacccatatacaangcagcagagcctggggtgcagagcagntntgcccagccccaaagcccacttccntntttccatagctcacagactcatagctcctnttnactntgctaacctcctacatgtattanattttttggggcctgaattagctccatgcttgaattgncctggacaaag

SEQ ID NO: 80 - DNA, 225 bp, Homo sapiens
gaagggagaagcagttcgtggagggagacgcgggaagaaaggggctgcgacaaagatggcggccgtgacggcacctgaggcggagagcgggccagcggcacccggccccagcgaccagcccagccaggagctccctcagcacgagctgccgccggaggagccagtgagcgaggggacccagcacgaccccctgagtcaggagagcgagctggaggaaccactgag

SEQ ID NO: 81 - DNA, 466 bp, Homo sapiens
cctagctctctttttcttgcagagacatctttctcccatctctgtctgttagtacagagctcttattcagccactagctcggcctttcctgcttcaattgtaatgcttgttctgcccggggacacactattgacagcagaaacaatgaatttcctccaaacccggcaatgttggtggctcttgcattcctctggatgagcgaatctagttggggggttcccgaaggggaaggcgcctgggctttcaatacatcctcctgaatcatactgcgtttcaggttccttagaaaaatttggatgtgtaaaaagaactcttaacggcgatgcaggtcttccacagctaaggtaggtgcagttttaagacgtgtctttcgcatattattatccttattttaaaaagccgtttaaacaatttgacttgcagtggctctccagcaaaggagggaaagcctcactggcgatatttg

SEQ ID NO: 82 - DNA, 392 bp, Homo sapiens
  misc_feature 15, 38, 161, 309, 310, 391: n = A,T,C or G
tctccatgaggagantcagggatccaggttgctaatancttgtgcttctgtcatcttagtaggtggccttcccaatggcatccacaggagaaaagagttgaaggatcacttggcagcttttccttacttcactgacctaggggtcactttgtagaacaggncccatgtctccacactgacttgacgtggggctaagaggcagtctccccatatgttcaggaagaagaaagtgaaacagccttactgaacacatagaagtgattttcctactgcaggcagtgattttcaaatttgttttaaagctgaggnntatttcagatgaaattttatgagaagctaagtatgtcagaggcataaaagttgcagtgcttgggtgaactggaacagaggnc

SEQ ID NO: 83 - DNA, 200 bp, Homo sapiens
gacaccatggctggagttttcgaaaagtggaactcatcttcctagcaacacaaaaaataattccagcatggtgggtaagtatggatgcttatcttaatcatgctagtatatgctgccatcaattctcctgcttgtcagcagtgaaactgcagctgtcaaatgaggaattgataagagacacgaggtgggacatacaatcc

SEQ ID NO: 84 - DNA, 491 bp, Homo sapiens
tttcactacaccagtggtgaccaaatagaagaggttcatccatacacagaacctggtgaagagctggaggcagaaagaagtgtctatgtggagacgcaactgaaacaaaggtggcacagcaactgttccaatcccgtgtctttcctcatggcttcccaggagtttgaggttgaagctattgttgacaaaagacaggataaaaatgggaatacacagtatttggttcggtggaaaggttatgacaaacaggatgacacttgggaaccagagcagcacctcatgaactgtgaaaaatgtgtacatgattttaatagacgacagactgaaaaacagaaaaaactgacatggactacaaccagtagaattttttcaaacaatgccagaagaagaacttccagatctacaaaagcaaactattctaagaactctcctaaaactccagtgactgataaacaccacaggtccaaaaactgcaagttatttgctgccag

SEQ ID NO: 85 - DNA, 150 bp, Homo sapiens
tagagcaggggctaacatggagaatcataattctgccttagctgctgaagctagtgaagaaagtttattttcagcctctaaaaatttaaatatgcctctaacaagggaatcttctgtcagacagataattgcaaattcatctgtccccat

SEQ ID NO: 86 - DNA, 190 bp, Homo sapiens
agttcacagaatggagcctggattttataggtcagaaattaccctcagagtacacatttaagggtcacagagtggtaatcatggatagagtaacaagtaagagttacagagtagggatctgagatggaagaccaatctgaggttcacaggtcagagttcagaagaaagaagaccaaactggagtcacagg

SEQ ID NO: 87 - DNA, 530 bp, Homo sapiens
tggaaacattctcaacagggaaaccctactagactttgtaaagcaaataatggaaaagatacagaactttttgaagaatcatgggaaatttttataattaaataaatgctaaaattctgttttgtgaaacatttatgggaattatcactgacagtttttgtacactttcaaatagtgttaaagcagcaactccatgttgtaaatgcacaaaacaaatatttagttaataatcaactccaagaataaagctgtaacaataatagttactattctctcatgtggtccatatttacctccacttagctggcatcttcaagtgccacatacttagcagcagcctggcaccttctgatattttgagaagcaggaatccctgagcataaatgtaaatagcttagaactgtccaaaagcaaagacagcagaaaataaaattgttgcttgctatgttcaggaaaggaatgcttccattggatatggaagccagtctcaattgttacatcagcctgaggaaactcatgcgagaaatgcc

SEQ ID NO: 88 - DNA, 335 bp, Homo sapiens; identical to SEQ ID NO: 7

SEQ ID NO: 89 - DNA, 423 bp, Homo sapiens
tctccggtatcctgatggagtctactaggtgtcagtcatttggccgtgcctcagccgaagagaggcggggaaaagcatcgtaatcagctgcgtcgccttttggtgacgccagagagtgcgcgtcagcagtttattagagagctctgtagccagcctcttctgcgcacccacctgctgcatcttagttcagtcggctcttagagtagtaaccgccagaaaggagtcggaagaggtctcacgaggctgtcatcaccgccatgcccaagaataaaggtactgctgtaagcctctgggactatacctcggcttgctctgccagtaaccccgacgcctgttccaggccgcagtgactgttctaacggcggtactggccactgcgaccccagcactgtgttcgggaaaggagctgggaatgccctattt

SEQ ID NO: 90 - DNA, 672 bp, Homo sapiens; identical to SEQ ID NO: 8

SEQ ID NO: 91 - DNA, 201 bp, Homo sapiens
aagcttctattcgagggcttcatgacccctgcaggatgagaagcaggtagtcatatttggcttctgcttggtaatctagcctctatttcatttcatctgcataggcttttcattgtggaggggttctttcattgggctgttgctagataaagctgtctctcaccacagattatttagatgtcagggattgcagagagcaaa

SEQ ID NO: 92 - DNA, 968 bp, Homo sapiens
aaccccatctctagcaaaaatatgaaaatttgctgggcatggtggtgcacacctgtagtatgttacagttaattggggggctcaggcaggagaattgtttgaacctgggagcctgaggctgcagtgagccaatattgcactatgtactctagcctgggtgacagagcgagactccaaatcaaaaataattatataaatctacaaatatgtaaataataaataaggtatccttcatttcaagcatttattctttctttttgcttttttagacacagggtctccctctgttgtccagcctggactgcagtggcaccgtcaaggctcactgcagcctcgaactcctgggttcaaatgcacaagccttccatttcagcctcccaagtagttgggattacagacacacatcactgtgcccaacttttgtgtttgtgtgtgtgtggtagggacaatgctttggatatattgttcaggctggtctcaatctcccagaccgaaataatcctccttccctggcttcccaaagtgttgtgattatagccgtgagccactgagtctggcatatcttttctcattatgagcgacattccacctcactgagtctggcgtatcttttcttggtatcagcgacattccaccttcgctctattaattattttgagatgtacaataaatcattattaagtgtagtcatcctgtgccactgaacactagatattattccttctaagcaagtataatttaacccacccccatcccctctttgatccctcgcttaccagttcacattacttgtatcaaaatatcacatgtatgccaaaagtatctacaactgttaggtacaaattttcattcccttcctccttccctcccttcctttcttccttcctgtctttctttctttttgtctctgtatctttttctctcactgatttttttttttttaagaaagaatcctgccctgtcaccta

SEQ ID NO: 93 - DNA, 401 bp, Homo sapiens
atctatcgacacttttgggaagtttcagccactattcatttaaatattctttttttccccatctcacctattccagtgggactcccattatgaatatgttgatattcttgatggtgtcccatggaaaccttagttttgatcattattcttgtttctcttccttacactgaacaatatcaaacaatccatcttcaagttgcctcattaggcattcctgaggcaggctaaggcacccttcagagctgtcagtctgcctaagcagaggaaaatggtacaggcagagccttcctggtatagggaaaaatgctgcctgtaataagccaccactggacacaaaaatatagatgatagggcccttttgggcagtctctaaggttgggttccacagaagaagtcatttt

SEQ ID NO: 94 - DNA, 453 bp, Homo sapiens; identical to SEQ ID NO: 10

SEQ ID NO: 95 - DNA, 601 bp, Homo sapiens; identical to SEQ ID NO: 11

SEQ ID NO: 96 - DNA, 601 bp, Homo sapiens
ctcacctcgacactgagccagtgatacagcataatctcatttgtatgctgggcctagtcagaagagtcacttcacccgggtacagtttcacataatatgtcagcaagcccactatggacagggaagaaaaaagagaggacagtcagtccacctatgtgctggactccgcaatatgtaacaaccctctctcttggtagagtctagaatatgaaggagagtcacatcatgtaggtttttcaatcagcggtatgtcacaatttgtttgctgagcaaggctcaggcaggaggtgagaatcacattacctagatgttaagccaaacaatatttcacaatgtcttctgggtgcaggacactgtcagaagagacaaatcctctagcttatagacccatagatatgtgataatatccccttttggcagggtccagacagaagagacacattatgattctaacacagtgatatgttacaatccacccaaggaaaggaatttaagccaaatagtctcaacacctaagcactatgcctattaataggccaaatccccttgtctttgagagtgacattataaactctgagctgggtgtgtatatgagagtaac

SEQ ID NO: 97 - DNA, 531 bp, Homo sapiens; identical to SEQ ID NO: 13

SEQ ID NO: 98 - DNA, 147 bp, Homo sapiens; identical to SEQ ID NO: 14

SEQ ID NO: 99 - DNA, 527 bp, Homo sapiens; identical to SEQ ID NO: 15

SEQ ID NO: 100 - DNA, 801 bp, Homo sapiens
tgggttagtggattttgggaggcagatgtggagatataaaatggggctagattatagagaatcttcaaagttagcaaagttaagattttgtgtaataggcagtagagagcttgtgtaagcttttttattcgagatgagccatcttgcagaccactgcttaagaagactgactgtggcagtttgcagtgtggaagggggaaagatggtgaaagctccaagtaggaggctagggctgccattcgtgggtaagatgaagaggaccaagacagtagagatgatatatagataattgtaaccaaatcaggagtaattgttggaacagcataacacatttacatatttgctgatgtaattactcaagggtcatatcataaaaaggggtgggggtttcacattatggaaaccttggaattctctgtcttacggtggaagacagaacccagtgtagtgtagacaaaagtctaattttaattcccagagaaagactgatatcttatgggacaaattagtatattcccattcctaataatggtgtacctagctggtgggtaattgcaggctttcaacctccagttcatcttcatgagatcaatgttgattcagaacaataagtgtcaggtctatgacacctttaaagttatgcttaaatgtgagtctgtgaccactaatgaatgactacagttgtttcatgacttgctaaacatacctggtttttcacaagcaatagatttttgaaggagaggtattcacagtgggtgctaggaagatacacacattaattctgtagaatttagctaaaat

SEQ ID NO: 101 - DNA, 340 bp, Homo sapiens
acctaagggaacccaggagaacagtgattcaaaattgaaaatatcaataaaaatgtatgaattattttgaaaaatgaaatcaataatctgtaactagaaggactgaatagcatgtgtgagcatgtagaaagcctaatgaggcaacttgaagatggattgtttgatattgttcagtgtaaggaacaagaagagaaacaagaataatgatcaaaactaaggtttccatgggacaccatcaagaatatcaacatattcataatgggagtcccactggaataggtgagatggggaaaaaaagaatatttaaatgaatagtggttgaaacttcccaaaagtgtcg

SEQ ID NO: 102 - DNA, 666 bp, Homo sapiens
ttgtgaaatggtggtgatggttgcacaatgtgagcatagttaatgccactgaactatacactcaataatgtttggagtggtaaattttttatgtatgtttttccacatacaacaaaagcttcctcagaagtcctcctaatagttccatgtgtgatgaaagaagacaattctctgttatcatgagtagaatcctatttatttattcatgctggtatacacactcctagctcttctacacgaacagcatgatctcatgaagcataatgagtcccttctcacctggcccatttgtgcttttttccatcattccattctgctaatcaactcactatctacccgctacaaaccactgtaaattacaggtctgtttactgtctctgtagacatttcttctctgttatgtaatagaaatgaaatcatacagtatgtcatcgcttcagaccagcttttatcacttagctttatgtatgcctgatacatccatggctttgcatggcttcataaatcattcctttcttttgatgaatagtatttccttttatgaatttacctggcctggttaatattgaagggcatcctgattccttaaagtatttggccattgtcaagagagcagttacacataaatgcctgtggttattgtttagggacgagtttccaaatcag

SEQ ID NO: 103 - DNA, 467 bp, Homo sapiens
ttaaggagcttgcctcatacaatccaattgtactggaagaatgtaaggccctcgttcgctgtaatattaagttggagttggaacaggccaatgagagagagtgtgaggtgctgaggaagatctggagctcagcccaagggatagaatccatgttaaagtatgttgaaaataaaattgatgagttttaattgtcagtctgtctgctcaggacacaagaactaaggggcaacaaatgcatcatgagttgcaagatgccctaatccatcttcatagcccaaaacaatttcacccatagctaaggcttggaaacagaactggaaatgtccaagctatgtatttaaattatcacatcatttttaagcactgtagctttacaaggagtaacaaaacagcctctttgcccaaatgtgattattttatgcacacctaagcccaaatataaaaacagactcttcttgcaagctcta

SEQ ID NO: 104 - DNA, 601 bp, Homo sapiens
ctgcgaagagaactcttccttacccttggtcacgataagtatctttctctacaaacaaactacttctgggctttctgtggcattttggatatctctgaacagcagttaaagagattggtgctctaacttcacgaaaatacaggaggcagagattgatctccttggttcccccgcttgctgtcctctacgttggttagaaaaatttggtcaaagatgaaatccacactgggaaggctcaggttgactttagaagatatgcttgattaaaaatggtttttttttttttttggttccattacattctatgaggaggcaaggaggcaggtcaacaggtagtgatcattcagcaaggtatattaatgccataaacaaacaacacagtacacaatggtcttgtgttaacctcacctatacaaactgagagaggcattgagcttatcctggtcaaattttttaatttagtgtgttaaaaaaatcttatagcataaacataaattaagaattttcctgaatagaagtcactggtcaaattggccacacatgtctcttctataaacaaaactccagaatatatggcaatgtctaaagaattcttggtaaa

SEQ ID NO: 105 - DNA, 677 bp, Homo sapiens
ccgacttccacaatggctgaactagtttacagtcccaccaacagtgtaaaagtgttcttatttctccacatcctctccagcacctgttgtttcctgactttttaatgatcgccattctaactggtgtgagatggtatctcattgtggttttgatttgcatttctctgatggccagtgatggtgagcattttttcatgtgttttttggctgcataagtgtcttctttttttttttttttttttttgagacagagtctcgctctgtcgcccaggctggagtgcagtggcgggatctcggctcactgcaagctccgcctcccgggttcacgccattctcctgcctcagcctcccaagtagctgggactacaggcgcccgccactactcccggctaattttttgtatttttagtagagacggggtttcaccgtttttagccgggatggtctcgatctcctgacctcgtgatccgcccgcctcggcctcccaaagtgctgggattacaggcgtgagccaccgcacctggccaagtgtcttcttttgagaagtgtctgttcatatacttcacccactttttgatggggttgtttgtttttttcttgtaatttttgtttgagttcattgtagattctggatattagccctttgtcagatgagtacgttgcagaacttttctccc

SEQ ID NO: 106 - DNA, 169 bp, Homo sapiens
atcgttttgaatggcgtcaaatgaaatggcatggaaggcagtgaattggagtggagtggaatagagaggaatgaattggaatggagtgtagtggaatagagtggattccagtggagtggagtggattggattggaatggaatggaatagattggattggagtggtgcag

SEQ ID NO: 107 - DNA, 236 bp, Homo sapiens
  misc_feature 46: n = A,T,C or G
tacgggtctcgaatggaataaaaatatatggaatggaatgcaatgnaacggaatcgaatgtcatagaatgtaatgcaatgcaaaaacatggaatccaaaatcattgactggaaaggctgggtgtcgaaaggaattgactccaatggaatggaatcgaatggaatggaagtgaatagaatcgaactaaatcgaatggaatggaattgataggaacggaatggaaaggaatgcaatga

SEQ ID NO: 108 - DNA, 461 bp, Homo sapiens; identical to SEQ ID NO: 17

SEQ ID NO: 109 - DNA, 20 bp, Artificial Sequence
  probe for STS
gaatattcccgctctccgga

SEQ ID NO: 110 - DNA, 20 bp, Artificial Sequence
  probe for STS
gctggtgctccattcttgag

SEQ ID NO: 111 - DNA, 24 bp, Artificial Sequence
  probe for STS
ttaaggggacagtatttcaacttc

SEQ ID NO: 112 - DNA, 24 bp, Artificial Sequence
  probe for STS
ccacatttaaactgagtacagtcc

SEQ ID NO: 113 - DNA, 20 bp, Artificial Sequence
  probe for STS
aacaagtgagttccacaggg

SEQ ID NO: 114 - DNA, 20 bp, Artificial Sequence
  probe for STS
gcaaagcagcattcaaaaca

SEQ ID NO: 115 - DNA, 21 bp, Artificial Sequence
  probe for STS
gaccaatttgtctttgttgcg

SEQ ID NO: 116 - DNA, 18 bp, Artificial Sequence
  probe for STS
gctgctgaagtcggcgta

SEQ ID NO: 117 - DNA, 21 bp, Artificial Sequence
  probe for STS
gggtcctagataggctccaag

SEQ ID NO: 118 - DNA, 20 bp, Artificial Sequence
  probe for STS
ttcatgttggcagtgattgg

SEQ ID NO: 119 - DNA, 20 bp, Artificial Sequence
  probe for STS
cccagtgaagaatcgtccat

SEQ ID NO: 120 - DNA, 20 bp, Artificial Sequence
  probe for STS
gctaacaagatgcactgggc

SEQ ID NO: 121 - DNA, 21 bp, Artificial Sequence
  probe for STS
cctaccgcatcagtgaatttc

SEQ ID NO: 122 - DNA, 22 bp, Artificial Sequence
  probe for STS
tctgtatgtggagtacacatgg

SEQ ID NO: 123 - DNA, 20 bp, Artificial Sequence
  probe for STS
ggtgtgctaacattgcatgg

SEQ ID NO: 124 - DNA, 20 bp, Artificial Sequence
  probe for STS
tttgttccatttcagagcga

SEQ ID NO: 125 - DNA, 20 bp, Artificial Sequence
  probe for STS
ggctgtgcaggttgtttcat

SEQ ID NO: 126 - DNA, 23 bp, Artificial Sequence
  probe for STS
gagattatgtcctttgcaggaac

SEQ ID NO: 127 - DNA, 20 bp, Artificial Sequence
  probe for STS
acctgtctgggaaacacctc

SEQ ID NO: 128 - DNA, 20 bp, Artificial Sequence
  probe for STS
gagccctacaaccagcttca

SEQ ID NO: 129 - DNA, 20 bp, Artificial Sequence
  probe for STS
tttttctaaccttgcctgcg

SEQ ID NO: 130 - DNA, 20 bp, Artificial Sequence
  probe for STS
tgcagagaagcagcctacaa

SEQ ID NO: 131 - DNA, 22 bp, Artificial Sequence
  probe for STS
tccttttccacaatagacgtca

SEQ ID NO: 132 - DNA, 26 bp, Artificial Sequence
  probe for STS
ggaagtatcttcccttaaaagctatg

SEQ ID NO: 133 - DNA, 20 bp, Artificial Sequence
  probe for STS
gactggagtggaacggtctc

SEQ ID NO: 134 - DNA, 20 bp, Artificial Sequence
  probe for STS
tcacttccctccgattttct

SEQ ID NO: 135 - DNA, 20 bp, Artificial Sequence
  probe for STS
gagattacaggcatgcacca

SEQ ID NO: 136 - DNA, 20 bp, Artificial Sequence
  probe for STS
ccacacttagcccacagtca

SEQ ID NO: 137 - DNA, 20 bp, Artificial Sequence
  probe for STS
aaggcaggtctgatgcatgt

SEQ ID NO: 138 - DNA, 21 bp, Artificial Sequence
  probe for STS
aaagaaagctgcctcatagca

SEQ ID NO: 139 - DNA, 20 bp, Artificial Sequence
  probe for STS
ttacccctttcacccactga

SEQ ID NO: 140 - DNA, 24 bp, Artificial Sequence
  probe for STS
ccataaactacacaaggacgaact

SEQ ID NO: 141 - DNA, 20 bp, Artificial Sequence
  probe for STS
ttgcacccgtagtcaaatgt

SEQ ID NO: 142 - DNA, 20 bp, Artificial Sequence
  probe for STS
acccacaactcaaatcgtct

SEQ ID NO: 143 - DNA, 20 bp, Artificial Sequence
  probe for STS
ctcttccaagccagccttta

SEQ ID NO: 144 - DNA, 20 bp, Artificial Sequence
  probe for STS
aacctttgcaagccacattc

SEQ ID NO: 145 - DNA, 20 bp, Artificial Sequence
  probe for STS
cagtgccccataacactttc

SEQ ID NO: 146 - DNA, 20 bp, Artificial Sequence
  probe for STS
atggtaatacagcagctcgc

SEQ ID NO: 147 - DNA, 20 bp, Artificial Sequence
  probe for STS
gaagggagaagcagttcgtg

SEQ ID NO: 148 - DNA, 20 bp, Artificial Sequence
  probe for STS
ctcagtggttcctccagctc

SEQ ID NO: 149 - DNA, 22 bp, Artificial Sequence
  probe for STS
cctagctctctttttcttgcag

SEQ ID NO: 150 - DNA, 20 bp, Artificial Sequence
  probe for STS
caaatatcgccagtgaggct

SEQ ID NO: 151 - DNA, 19 bp, Artificial Sequence
  probe for STS
atcacttggcagcttttcc

SEQ ID NO: 152 - DNA, 20 bp, Artificial Sequence
  probe for STS
gcactgcaacttttatgcct

SEQ ID NO: 153 - DNA, 20 bp, Artificial Sequence
  probe for STS
gacaccatggctggagtttt

SEQ ID NO: 154 - DNA, 20 bp, Artificial Sequence
  probe for STS
ggattgtatgtcccacctcg

SEQ ID NO: 155 - DNA, 22 bp, Artificial Sequence
  probe for STS
tttcactacaccagtggtgacc

SEQ ID NO: 156 - DNA, 20 bp, Artificial Sequence
  probe for STS
ctggcagcaaataacttgca

SEQ ID NO: 157 - DNA, 20 bp, Artificial Sequence
  probe for STS
tagagcaggggctaacatgg

SEQ ID NO: 158 - DNA, 20 bp, Artificial Sequence
  probe for STS
atggggacagatgaatttgc

SEQ ID NO: 159 - DNA, 20 bp, Artificial Sequence
  probe for STS
agttcacagaatggagcctg

SEQ ID NO: 160 - DNA, 20 bp, Artificial Sequence
  probe for STS
cctgtgactccagtttggtc

SEQ ID NO: 161 - DNA, 21 bp, Artificial Sequence
  probe for STS
tggaaacattctcaacaggga

SEQ ID NO: 162 - DNA, 20 bp, Artificial Sequence
  probe for STS
ggcatttctcgcatgagttt

SEQ ID NO: 163 - DNA, 20 bp, Artificial Sequence
  probe for STS
aggaatcatttgaggccctt

SEQ ID NO: 164 - DNA, 20 bp, Artificial Sequence
  probe for STS
tggagttcgggttcaaattc

SEQ ID NO: 165 - DNA, 20 bp, Artificial Sequence
  probe for STS
tctccggtatcctgatggag

SEQ ID NO: 166 - DNA, 20 bp, Artificial Sequence
  probe for STS
aaatagggcattcccagctc

SEQ ID NO: 167 - DNA, 20 bp, Artificial Sequence
  probe for STS
gcacctgccacgcatatagt

SEQ ID NO: 168 - DNA, 20 bp, Artificial Sequence
  probe for STS
gcaaacatgctcacgatcac

SEQ ID NO: 169 - DNA, 20 bp, Artificial Sequence
  probe for STS
agcttctattcgagggcttc

SEQ ID NO: 170 - DNA, 20 bp, Artificial Sequence
  probe for STS
ctctctgcaatccctgacat

SEQ ID NO: 171 - DNA, 24 bp, Artificial Sequence
  probe for STS
aaccccatctctagcaaaaatatg

SEQ ID NO: 172 - DNA, 20 bp, Artificial Sequence
  probe for STS
taggtgacagggcaggattc

SEQ ID NO: 173 - DNA, 21 bp, Artificial Sequence
  probe for STS
cgacacttttgggaagtttca

SEQ ID NO: 174 - DNA, 20 bp, Artificial Sequence
  probe for STS
ttgtgtccagtggtggctta

SEQ ID NO: 175 - DNA, 22 bp, Artificial Sequence
  probe for STS
tcatttgtgtccttctcttgga

SEQ ID NO: 176 - DNA, 20 bp, Artificial Sequence
  probe for STS
ctaagccaggaacttgccac

SEQ ID NO: 177 - DNA, 20 bp, Artificial Sequence
  probe for STS
ccagacgttctaccctttcg

SEQ ID NO: 178 - DNA, 20 bp, Artificial Sequence
  probe for STS
gagccgagatccagttacca

SEQ ID NO: 179 - DNA, 20 bp, Artificial Sequence
  probe for STS
tcagtccacctatgtgctgg

SEQ ID NO: 180 - DNA, 20 bp, Artificial Sequence
  probe for STS
attcctttccttgggtggat

SEQ ID NO: 181 - DNA, 20 bp, Artificial Sequence
  probe for STS
tgatatcaggtgatgcgtcc

SEQ ID NO: 182 - DNA, 20 bp, Artificial Sequence
  probe for STS
tggcgttaagtaaacctggg

SEQ ID NO: 183 - DNA, 20 bp, Artificial Sequence
  probe for STS
gggtgttaccagaaggcaaa

SEQ ID NO: 184 - DNA, 22 bp, Artificial Sequence
  probe for STS
gaaccgtatctaccaaagcagc

SEQ ID NO: 185 - DNA, 20 bp, Artificial Sequence
  probe for STS
taaaaggcagaactgccagg

SEQ ID NO: 186 - DNA, 20 bp, Artificial Sequence
  probe for STS
gggagaaaagttctgcaacg

SEQ ID NO: 187 - DNA, 20 bp, Artificial Sequence
  probe for STS
gtgggggtttcacattatgg

SEQ ID NO: 188 - DNA, 23 bp, Artificial Sequence
  probe for STS
ggtcacagactcacatttaagca

SEQ ID NO: 189 - DNA, 20 bp, Artificial Sequence
  probe for STS
acctaagggaacccaggaga

SEQ ID NO: 190 - DNA, 21 bp, Artificial Sequence
  probe for STS
cgacacttttgggaagtttca

SEQ ID NO: 191 - DNA, 20 bp, Artificial Sequence
  probe for STS
ttgtgaaatggtggtgatgg

SEQ ID NO: 192 - DNA, 20 bp, Artificial Sequence
  probe for STS
ctgatttggaaactcgtccc

SEQ ID NO: 193 - DNA, 24 bp, Artificial Sequence
  probe for STS
ttaaggagcttgcctcatacaatc

SEQ ID NO: 194 - DNA, 24 bp, Artificial Sequence
  probe for STS
tagagcttgcaagaagagtctgtt

SEQ ID NO: 195 - DNA, 20 bp, Artificial Sequence
  probe for STS
attgatctccttggttcccc

SEQ ID NO: 196 - DNA, 20 bp, Artificial Sequence
  probe for STS
gacatgtgtggccaatttga

SEQ ID NO: 197 - DNA, 18 bp, Artificial Sequence
  probe for STS
ccgacttccacaatggct

SEQ ID NO: 198 - DNA, 20 bp, Artificial Sequence
  probe for STS
gggagaaaagttctgcaacg

SEQ ID NO: 199 - DNA, 20 bp, Artificial Sequence
  probe for STS
atcgttttgaatggcgtcaa

SEQ ID NO: 200 - DNA, 19 bp, Artificial Sequence
  probe for STS
ctgcaccactccaatccaa

SEQ ID NO: 201 - DNA, 20 bp, Artificial Sequence
  probe for STS
tacgggtctcgaatggaata

SEQ ID NO: 202 - DNA, 20 bp, Artificial Sequence
  probe for STS
tcattgcattcctttccatt

SEQ ID NO: 203 - DNA, 20 bp, Artificial Sequence
  probe for STS
agtcggagtcggagtgtgat

SEQ ID NO: 204 - DNA, 20 bp, Artificial Sequence
  probe for STS
attccattgctttccattgc

SEQ ID NO: 205 - DNA, 432 bp, Homo sapiens; identical to SEQ ID NO: 1

SEQ ID NO: 206 - DNA, 362 bp, Homo sapiens
gtgggtgcatgatgtactgcgttggattcagtttgccagtattttattgagaatttttgcatcaatgttcatcagggatattggcctgatgttttcttttttgttgtgtatttgccaggttttggtatcagaatgatgctgacctcatagaagaagggagtggtttctccttttcagtagtttggaatagtatcagaagaaagggtatcagcttttctctgtacttctggtccccagaataatggtccccaactccatcccagttactgtgaatgccattatttcattcctgtttatggctgagtagtattccatggtataaatgtatagcacattttctttatccactcgttggttaacgg

SEQ ID NO: 207 - DNA, 385 bp, Homo sapiens; identical to SEQ ID NO: 65

SEQ ID NO: 208 - DNA, 508 bp, Homo sapiens
aggctactgtgaatcacgccactgcactccagcctgggcaacagagtaagaccttgtttctatctctgtttctctctgtctctctcacacacacagacacacacaaataaagggaaagatattcattaacaatctccagttgtaattgctttataatgatatcttaatcttgaaatgaattacttagatttacataaaataggcacatctagctgtggtaaggatacactgttcatttagtatggagccagcacagaaatgggagaaagacagttgtagagaaactgtcaagggaattcaagtccccaaaaccccagctaagcagcgtgaagaagtagaatagctgggggaatggcgcagcttgctgttttttggtgctggttatgaccaaaccttagaaaaagcatcatactgtgatgaaatcagccacatagaccagtggaacagaagagagagcccacatgtattcaactactttttgacgagggcacaaagggaacacatgcac

SEQ ID NO: 209 - DNA, 440 bp, Homo sapiens
cgtcggtattttacgacacggaagaatataacatatacatcggtattttacgacaccgacgattataacagatacgtcggtattttacgacaccgacgaatataacatatacgtcggtattttacggcacctacgaatataacatatacattggtattttacgacaccccaccccccaaaaaaaggcgtcacatttacataaacataattatcttaaaagccagtataattttaattttattgtagtcatcaccttcagactttattttggagaagtgattacggaaatctgaaatatcaaggcctgatgagaacacttaaattaaccacactccagaagtccaaatctgaaaagcaaagatgtttctgatataatagtctaaattctgcatttcctctctattgggcagtatgatattgcgcacatgaaaaacaaatgc

SEQ ID NO: 210 - DNA, 416 bp, Homo sapiens
tacacctccgaagactgaaccaggaagaagctgaatccctgaatacactaataacaagttctgaaattgaggcagtaataaataatctaccaaccaaaaaagctcaagaccagatggatttcccgatccatcttctattctattctattctattcattattctaattgaagcaacacaataattataaaccaaacacaggatgttctcacttagaagtgggagctatgctatgaggatgcaaatgaataagaatgatgtaatggactctgtggactcaagggggaaggtgggagggggtgagagataaaagggtacacattgggttcagtgtacactgcttgggtgatgggtgcaccaaaatcacagaaatcaccactaaaaacttatccatgtaaccaaacactacctgtttccc

SEQ ID NO: 211 - DNA, 298 bp, Homo sapiens
ccagacgttctaccctttcgagattagttaatatgtttacacacagagttttctttataggattataatttacaatgttttcacaattttcttaaacagtccactttattttatttaactttaagacaacttttttattcttaagcaaaatacatagttatgccttataatttttaactaaaaccactttttaccatttttatacacttttatgcaaatccatgtttagcagtagtgggcgcctgtattcccagctactccagaggctgaggcaggagaatggcatgaaccaagggag

SEQ ID NO: 212 - DNA, 834 bp, Homo sapiens
tcccctcagccatctgtattctggatttcaggatatcacttttagcctgtgatcccaacatggactacatgggatcacaggctaccccatgggtatcatgactaccccatgtgtttactggcactctttaaaccataatttgattgaacgggaaactgagtagttttcaattaagtacctacttgcatttttaggccattgctgatgaaacagatttttttaaatagtcattagcaaatagcccataaataggtagttataagaatataacagatgacaaaacgactgtggccagacaaatcaactactgtctgtcacctaagcttctatttccaacacataatttacatatatctagcctattatttccttccttttccccagacctaccatttccacaaggggcaaataagaaaaagaatttaacaccccccatataaagtacaaacagtaatctgaaaagaaccacaagggaaaaaaattcaaaatttacaactatctaccctaaaagaagctgaaagtccctcaaaaactttctagaggccatgtccttctattacaaaaatgatcataagaactgcaggagtagaagaataaaaatgcatcttaaaacttgctaaacacttcaagtctcccataagaattgtaatggaaaatggatcagtcagcagttttttccatacaattatgaacgaattatatttcctcatacacagatttgttttttcaatattctaaggaattaacttttatactaatagtaggtgatgtaagaaagcaggcctttatcaagataactgacactggatgtccataccattactcaggtgggcc

SEQ ID NO: 213 - DNA, 493 bp, Homo sapiens; identical to SEQ ID NO: 71

SEQ ID NO: 214 - DNA, 493 bp, Homo sapiens
atctgcacacttgggtaggcaaggcaggtagattaccagatgtcagaagttcaggaccagcctggtcaacatagtgaaaccccatctctactaaatattcaaaaattagccaggtatggtggcaagtgcctggaatcccagctactcgggaggctgaggcaggagaattagttgaacccatgaggtggaggttgcagtaagccaatatcagaccactgcaatccagcctgggccacaagagcaaaactttttctccaccccacccccccaaaaaaggcgtcacatttacataaacataattatcttaaaagccagtataattttaattttattgtagtcatcaccttcagacattgtttattttggagaagtgattatggaaatctgaaatatcaaggcctgatgagaatacttaaattaaccacactccagaagtccaaatctgaaaagcaaagatgtttctgatataatagcccaaattctgcatttcctc

SEQ ID NO: 215 - DNA, 500 bp, Homo sapiens
gctacttgtgaatcacgccactgcactccagcctgggcaacagagtaagaccttgtttctatctctgtttctctctgtctctctcacacacacagacacacacaaataaagggaaagatattcattaacaatctccagttgtaattgctttataatgatatcttaatcttgaaatgaattacttagatttacataaaataggcacatctagctgtggtaaggatacactgttcatttagtatggagccagcacagaaatgggagaaagacagttgtagagaaactgtcaagggaattcaccaattcctgcctcccagcagctgggacagagggttacatggccagggcataatccacactccgcagaaggaatacacatgtggaatagagtgagattagtgttaccactctcctatttctaaaatctaattttaggtttttaggaatatgcttgagaaattcagtatcttcaattctgagacaatgcttcgaaatatgca

SEQ ID NO: 216 - DNA, 578 bp, Homo sapiens
tgctcctccttctcacgtttctcctgccgctctaatcccgccttggccatgaaggaaatcgtgctcacgcaggccgggcagtgcgggaaccagattggcgccaaggttgtatttgactccctaaaatttaaaattttgtatgagagaaaaatacctcaaagtcaaaagtcaacagactggggaaatagatctgcaacatacatgacagacaaaaagctaatttgggatatatatatacacatatatatacacacacacatatatatacacacacatatacatacatacatataaatatatatacatataaatatatatagagatagatagatatatgtaaatatccattttttttgtttttttttttttttggtgaacaagtgaaaggaagtttattgtgtttggtacatatatctgtttctgttttccatacaagcacatcaaccacaagacttacagagcacaatggaataataatcacaaaatatttaaagatataaaaagacctaacaattccaatcataagcaagtccctcatacaggacataaaccacaaaagcaacaagacatgtacggga

SEQ ID NO: 217 - DNA, 420 bp, Homo sapiens
ccatcctcacgacttggactcctggctcatcctcgtgaggcccggaggccgctttctacctacatggttgatcctaccagaaccatatgcttgtctcaaagattaagccatacatgtctaagtacgcagggccggtccagtgaaactgcaaatggctcattaaatcagttatggttcttttggtcactcgctcctctcctaattggaaaactgtggtaattctaaatctaatacatgccaaagggcgctgacccccttcgcggggaagatgcgtgcatttatcagatcaaaaccaacccagtcagcccctctccagccccggccggagggtcaggtgccaccagcttttgtgacactagataacctcaggccaatggcatgcccccagtggcagcgacgacccattcaaaaatctgcctt

SEQ ID NO: 218 - DNA, 312 bp, Homo sapiens
tggaataccctggattggaatagagtggaatcgaaaggaatggaatggaatggagtgcattggagtggagtggattggagtggaatggaatgggacgaaatggaatgcagtgtagtggactggagcactttgacgcctatggtggaaagggaaatatattcacataaaacctagacagaagcattctgagaaacttctgtgtgatttgttcattcttctcacagagttgaacctttctttttattgaacagattggaaaaactctttttgcagaatctgcaagtggacatatggagcgatttgaggcataag

SEQ ID NO: 219 - DNA, 422 bp, Homo sapiens; identical to SEQ ID NO: 4

SEQ ID NO: 220 - DNA, 216 bp, Homo sapiens
ttgactgcttattctacacaaggcaatttcacattaattaatgcatgtaatcctcatagcttctctgtaaagtagacactggttttcctcattttacacatgaagaaaccaaatcacaggaacatctccatgatgatattggaacaggaattaaaagaaattaaagagtgtgtaagcaaaaactcagttgtttgtaggaaaacccaatttcccctg

SEQ ID NO: 221 - DNA, 110 bp, Homo sapiens
gggtcggtgcacctaaataattaaataattcctcctcaacccctaggtctctctgattccttaattatcctgctgcaaatactcagaatttattatcactctttagtgca

SEQ ID NO: 222 - DNA, 239 bp, Homo sapiens
tcaggtactgtgatgccgttagttttgtttgttttgctcagagttgctttggctctttggtctctatcagtttcatacaaattttagtatttttttttctatttctattgaaaataacattgatattttgatacagactgcattgaatctgtagatggcttctagtagtatggtcattttaatgatgtcaattcttttgatcaatgacatgcaatgtttttccctttgtgtcctcttca

SEQ ID NO: 223 - DNA, 131 bp, Homo sapiens
tcacagctcagttctttgggtgttaatctgactgggtcggtgcacctaaataattaaataattcctcctcaacccctaggtctctctgattccttaattatcctgctgcatttcctcatccaacatggaca

SEQ ID NO: 224 - DNA, 567 bp, Homo sapiens
cctttttgcaactgtggagacaagaagtagatttcatatttccatatattagaaatacacaggacttttctgtatatagtagaatttactatatacaaatatacataaatatatgtaagtaaaatatacaattatatttattgtaggcaaacctatgtatttatatatgaaatatataaatacaaacatacaaatatggaatttatctttatatataaaatacataaatatgaatatatatgaatatatattatatatacacactttagtaatacacaggtcttttctatgtatactagaacctaccatatatttatatattaacatgtattttcatatttttatatttatatatagattgtatatagaaatattgatatatagatatatattttatatataaatatctatataaaacatatttatatgtatatattatatataatcatttattttagaagttaaggtgttatgattgttgaagcatggttcacaggcaaaaagagaaaggaaattatcacttttagaagaataaagtacaaaattatcacgatggaatacccagct

SEQ ID NO: 225 - DNA, 349 bp, Homo sapiens
atctagaattgggccttgccttcccactagccacaacatcagtactctctgcattcatgtctctgtcctaatgatatcttactcactaagggaggaagggcttaccttatcttatcctgcttctggccccataagtgttattccataaaatcttgttaacaacagaattgggatttaaatctgttttgaaacctagttaatatgtcttgcccacctgtctatgtcataaacaaagacaactattattaaaacaacaacaatacaatgtttcttacttccaaacacaaatacgtatccattcaaaaaactagtaattaagtatgcactacacgttaggcaacgttcccca

SEQ ID NO: 226 - DNA, 335 bp, Homo sapiens
tgaaatgtaaaaccgtggacatttggaaaagaaaatgatcctacacaatatttattaaaacttcctccctgagattgtttttccaaaaataagtaatgtgtgtggcaaaaaagttaagagaatttcagggttacaatcttgttaacaacagaattgggatttaaatctgttttgaaacctagttaatatgtcttgcccacctgtctatgtcataaacaaagacaactattattaaaacaacaacaatacaatgtttcttacttccaaacacaaatacgtatccattcaaaaaactagtaattaagtatgcactacacgttaggcaacgttcccca

SEQ ID NO: 227 - DNA, 554 bp, Homo sapiens
agggtcccctccctacaatctaatataattaagaagaaaaattcaaagtagagcaatatctatctacttgaaaaaagcctttttgcaactgaggagacaaaaagtacatttcatatttccatatattagaaagacacaggtcctttatatagtagaattcactgtatacaaatgtatagaaacatatgtaaataaaatgtaatatacaactatatttactgtatacaaagctatatataatatatatttacatataaatatataaatacaaatataggaatatggattatatatttatatgtaatagaaatataaatatatattgatatagatgatatatatctgttagaaatacgcaggtcttttctataaatactggaacctactatataaatgtatatattaatatatgtgttttcttatgatttgtagattatatatagaaatatttatatattttatgtataaatacatatataaacaattattcatatatgatatataatcatttattttagagggtaagctgttagtattgttcaagcaggggtctc

SEQ ID NO: 228 - DNA, 500 bp, Homo sapiens
ggccccgtaggtctatacaaagccaacttctgtgaaaaacagtgttgatgcttcaacagtgattcctattcttggctccacattagaatgacccaaggagctctttgtgtaaaatgctgatgtaaaggacacctccttaaagcaataaaaccgatgctgaccttgatttacaatgtgcagctatagttgagagccacttgcctagaggagatcttctcaatcttaaatctgcatgggaacttccttcttatcttgttaacatgtagggtctaattaactcagtctgtggtgaagacttgaaattctgcatttctaacaacctccttggaggacaaagctgaccagtcctggagtagtgtggttgagttttggcaacattgagattactcacacaactcgatttttaagtgcttcagtatgtgtttatatgtgtcctctttttatctttattaattattcacagagcattcaacagaacaccaacatggcaagtcatgacc

SEQ ID NO: 229 - DNA, 438 bp, Homo sapiens
tccaagcatatagttgcccccaattctttaatttcttctggctagaaaagccataggctttctatatgaattttagctgaagtgttaattgagatgtatattctggctaaaatctgtaagaatggaaacttcaaaattgtggtttgttttatccaggtaccaacttccctccaaaatcagcccgcttgtatttactttcttctgacttcagatagttgatttgcgtctcttccccagattttattcttattacctgagcatttgttggtgcagtattggcttcactggacacacagaagtgaaattcctgaactgaacttctacagaatttgatacttaattaagcaaaaatcagagagtgatgaactacatgcaagataaatatgcctatcagagattctggggcacattcgagtgtgctccatgtgcagtgaaaga

SEQ ID NO: 230 - DNA, 439 bp, Homo sapiens
agaaaacttgcaatgcaggagtaggtttatgtttcctctgcatcagtcatttgagagaaatatctttaaatatgaaattgcaaaaccgaatcctgtttgtgtctttgtctttgataatgttcaaatatcaaggctcataagtatgatcttcaacaaaaaggagcataatggaaaaaactgcaatatcttttgaagatttttaactcaggctgggcacagtggcccatgcctgtaatcctagcagttctggagggcaacctgggtgaatctgctgaggttagaagtttgaaatcagtttctcttgtggtttgttttatccaggtaccaacttccctccaaaatcagcccgcttgtatttactttcttctgacttcagatagttgatttgcgtctcttccccagattttattcttattacctgagcatttgttggtgcagt

SEQ ID NO: 231 - DNA, 432 bp, Homo sapiens
gctccacattagaatgacccaaggagctctttgtgtaaaatgctgatgtaaaggacacctccttaaagcaataaaaccgatgctgaccttgatttacaatgtgcagctatagttgagagccacttgcctagaggagatcttctcaatcttaattctgcatggaacttcattgtatttctcacatatatcacatgttcatgggggaaaaaatgaaaaagaattgtgaactataaagtactccacattttacatacaaaaacatcctgaatatatgttgaggttagcaggaagaaatcatctcttactccttaagattggaatctacagagggaagaataccttatgtcttaaaaataatattttggacacatagcaaatttcaaatccagctttattttttgaagaattaaatgggggtcacaatatttttcc

SEQ ID NO: 232 - DNA, 402 bp, Homo sapiens
ttgcaacattgcacttgtaggaatttggagatgtcctaagtggttaactctattgaaagagtcacataagtgcataggatatttctgctctctgatgacctatagctatttagacaatgtagataaatatacaggctcagccacaacgtgttctcaagatattttgtttggcaggaaactacatttcagggcattaactacaatgcatgtaacggggtgaaatagttgaaatgatgctgaaagaggaattttgctttcctttctttaatttgttatcagtagtatactgtttgatatcagtttcaactataagagcatgctggggaaccgaattgcctttgaaatagtgcccaggttttgagagtagctcttgggtagcacaggcaccaagaccagaatcat

SEQ ID NO: 233 - DNA, 428 bp, Homo sapiens
cctatctctgcatggcctgttttttcatagcttatgattatagagcaaggattaatacagtattggaataaagagtaattgttacaaactaacgattaatgatatccatatacgatcatatctatgatctatgtctagtataactcttgttgttttatacagtttattataatagaacagctcacgccctctgtctcttgcctctgaaactaggtggcttgctacccacactctcccacaacccatgggaattgtgggagccacaattcaagatgagatttgggtggggacacagacaaatcatatcaaaggttaaacagcacacttgttcttcaagttgcccacttgagtcttttccaagcatactttcctttttttcgtgtcttaaagcctttttaaataaacttctctgctccaaaacttgcctt

SEQ ID NO: 234 - DNA, 343 bp, Homo sapiens
atctcacatggtgccagacaagagaagtgaacgtgtgcagggaaactaacctttataaaataatcagatctcatgagtcttattcactaccatgagagcagcatgggaaagaccgactcccatgaattaattacttccactggggccctcccacaacccatgggaattgtgggagccacaattcaagatgagatttgggtggggacacagacaaatcatatcaaaggttaaacagcacacttgttcttcaagttgcccacttgagtcttttccaagcatactttcctttttttcgtgtcttaaagcctttttaaataaacttctctgctccaaaacttgcctt

SEQ ID NO: 235 - DNA, 403 bp, Homo sapiens
tgcacttgtaggaatttggagatgtcctaagtggttaactctattgaaagggtcacataagtgcataggatatttctgctctctgatgacctatagctatttagacaatgtagataaatatacaagctcagccacaacgtgttctcaagatattttgtttggcaggaaactacatttcagggcattaactacaatgcatgtaacggggtgaaatagttgaaatgatgctgaaagacaaaagtttctgctgtgccagaaactttctgagccgctatagtttagggctgtctgattttggaatctttgatcaattaaactctgttaaatttaatatactgaaagtttttattttaacaatagtatacatatatatatacagtgtatgtgtatatatgtctgtttt

SEQ ID NO: 236 - DNA, 790 bp, Homo sapiens
ggcttaaacttgggagggtgtatgtgtccaggaatttatccatttcttttagattttctagtgtatttgcatagaggtgttcagagtattatctgatgatcagcaatgatattccctatgtcattttttattgcatctatgtgatttttctctcttttcttctttgttagtctggctagtggtctatctatattgttgatcttttcaaaaaaccagcttacggatttattgatttttgaaaggtttttgtgtctctgtcttcttcagttctgctctgatcatagttattttatgtcttctgctaggttttgtattcgtttgctctcactaaactagttcttttaattttgatgttagggtgtcaattttagatctttcctgctttctcttttgggcatttagtgctatcagtttttctctaaacaccactttaaatgtgtctcagagtttgtggcacattgtggttggtggcctggagagacattggtcaaaaatacaaacttttagttaaacaggaagaataactcaaagaaatctattgtacaacatagtggcatcacactatgttaagggtctaacactacctgatacaatgttatatctaataacaacgtattgtactcttgaaaatggctacaataatagatttaagtgttctaacattaaataaatgaaaagtatataatgtagtgcatgttaattagcttgaaatgaatacatatttcaaagaggtcttaaaaagcaccatcctagacatttaattataagaataagcctcggtttgaagtgc

SEQ ID NO: 237 - DNA, 318 bp, Homo sapiens
gcaaagttcttgcactgtgttttttagctacatcaggacatttatgttttcctctaaactagttataatcataattattactatttttgagacggagtctcactctgtcaccagcctgaagtgcagtggtgtcaactcagctcacttcaacctccacctcccgtggattcagctgctcttagctgagactacatagcgggccatcttggcaccctaaagtgaagcctgagctgtccaaccatggagtaaatgtagagagcctaatcgacatagtatagttgcttcttcacacaactctttctacaccacaggccacaa

SEQ ID NO: 238 - DNA, 386 bp, Homo sapiens
gcaaagttcttgcactgtgttttttagctacatcaggacatttatgttttcctctaaactagttataatcataattattactatttttgagacggagtctcactctgtcaccagagagcttctggaaatctgaacttatagattatcctagacaattgtgtgcatataaaaccagacattaatcattttaaacgtctaagaatatgtacgtgtttttcctttgttttgaagcaagtttctgcatttaatcatgtgtaaagaattttagcttattaaatagaatataatctaagttaatctaaaaagcacactttatgaccataaaaacttagatatttctacctaaatgattccatgattgactaatcctgctcaaggtttccttt

SEQ ID NO: 239 - DNA, 314 bp, Homo sapiens
ggttttgtattcgtttgctctcactaaactagttcttttaattttgatgttagggtgtcaattttagatctttcctgctttctcttttgggcatttagtgctatcagtttttctctaaacaccattttaaatgtgtctcagagtttgtgacacattgtgtcttcattcttattggtttcaaagaacatctttatttctaccttatttcagtatttacccagcaatgatttacagcctgctcctaaatgttcagtttctatgtagttgtgcaggtttgtgtgagtttctgaatcctgggttctcatttcattgca

SEQ ID NO: 240 - DNA, 302 bp, Homo sapiens
gtcaggaggcttaggaaggctaagtaaaaatattttaaatctgttttgtcagagactaggaatgcaatttccctttatttctttccttaaaactatcgctagtgtgactgaataactgaatttttaatctatgcttaataaactacatgtgcataatgactaccatacagggtagcataattctaaggtacatggctggtatctgttgcttaactcttactaccaaaggaaatttctggcttgaagggatattaagaaacaatctacgggccacgcatgatggtccttgccagtaatcccag

SEQ ID NO: 241 - DNA, 318 bp, Homo sapiens
gtgctgttaagcaccatccagtatgactcgctgtgaaaattgaaatgtttgtatctatgttgttgactgtggtaggtactagcctcatgtggctattgagcacttgaaatttggctagtgtgactgaataactgaatttttaatctatgcttaataaactacatgtgcataatgactaccatacagggtagcataattctaaggtacatggctggtatctgttgcttaactcttactaccaaaggaaatttctggcttgaagggatattaagaaacaatctacgggccacgcatgatggtccttgccagtaatcccag

SEQ ID NO: 242 - DNA, 320 bp, Homo sapiens
gcaacatagatggacccagaaaatattatgttaagtcaaataagccaggcacacaaagacagatactacataatctcatttatacctagaaataaaagcagtcaaacttacagaagcatagagtaaaggggtggttaacagtaaaggttcattggtttcaaagaacaactttatttctaccttatttcattatttacccagcaatgatttacagcctgctcctaaatgttcagtttctatgtagttgtgcaggtttgtgtgagtttctgaatcctgggttctcatttcattgcactgtggtctgggaggctctttgctat

SEQ ID NO: 243 - DNA, 461 bp, Homo sapiens
ccatctcctgacctcgtgatctgcccaccttggcctcccaaagtgctgggattacaggggtgagccaccacgcccaggcatagaggcacttttaaccataaatgaacactgttatgatttgtattaccacagtatcattattctgtcctgtttgccttacattttatttatttattatactgtaagttctgggatacatgtgcagaatgtgcaggtttgttacagagatatatgcttgtttgctgcacctgtcagtttttcatctacattaggtatttctcctaatgctattccctgttaggtccccaccctccaacagtctccagtgtttgatgttcccctccctatgtccatgtattctcattttacaactcccacctatgagtgagaaattgcagtgtttgtgtgtttggaacttattccttccagtgggtttgtggtctcgctcactgcaaaaatga

SEQ ID NO: 244 - DNA, 370 bp, Homo sapiens
ctggcaggttcatggtctttctcacttcaagaatgaagctgcagaccttactggtgagtgttgcagcacttaaatgtgttatgtccagggtttgttccttcatatgtgtccatagtttcttccttctggctggttcatggtcttgctcacttcaagaatgaagctgcagaccttagtggtgagtgtctgctaaaaacaatgtaaaaatatgtgtctgctaaaaacagtttaaaagtgttcctctttctccacagccttgccagcatcagttgtttcctgaccttttaataatctccactatgactggtgtgagatgatctctcatcgtcgttttgatttgcatttctctgatggttggtgatgttgggct

SEQ ID NO: 245 - DNA, 267 bp, Homo sapiens
tcgggttcaacagattctcctgcctcagcctcccaaatagctaggattacaggaacctgccaacacacagataatttttgaatatttagtagagatgaggtttcaccatgttgaccaggctggatttgaacttctgacatctggtaatccaccagccttggcctcccaaggggattacaggagtgaaccaccccgtgtgtggttaaaacatatacataaacatatacataagaactgtttacaaaaattgctgtggcctgtagaaaa

SEQ ID NO: 246 - DNA, 268 bp, Homo sapiens
caatggcattcactgtgaacactagacaaagctcacagctcaggcctggtagcttgagaagagcgcatgtgcattcggaaggcaggcacaggcgcccaaatatcagatctgtgagccttcctaagcagaggaaaatgctacaggcagagccagcctggtatctggagaaaggctttctgcgataacgcactatgggaccctgtaagtctcaaccttagggctccgtcgggccatttttgtggcctggtccagctggaggagtttcaag

SEQ ID NO: 247 - DNA, 489 bp, Homo sapiens
catgcagaagatcatccacgtactggagcaaaacgcagcataggtctttggtggggaacttttggaggtctcaagccagcacctctcctgaagatggtgggtgagtgcttaaacccttggagaagcaggattcaagtgtactgagtggtgacacctaaccccggatctttcttcccactggaaggcaaacagtttctggctctcaggagatagtctgatgctaaataaggtgtccttcaggtctaggcaagtgaaccacctgtcctcagctgacaataacccaacaatgtgtatgggttaggtactgttggatgtaaagtcactgtagtgtgactgaccaagcacaagtcctgtacttgcctgtagtcctcagtccctggcttaggaacaggcaggaggggagtgttccatggcgactgacaagggactataattccaaaggctctgaggcatttgagatgaacctggctaccttcaataacttgtctg

SEQ ID NO: 248 - DNA, 209 bp, Homo sapiens
gcactgaaggtgtttcacgatggtgatggatccaggctgtgtatgggttaggtactgttggatgtaaagtcactgtagtgtgattgactaagaacaagtcctgtattggcctgtagtccttggtccctggcttgagaacaggcaagaggggagtgttccatggtgactgacaagggactataattccaaaggctctctggcatttgaga

SEQ ID NO: 249 - DNA, 183 bp, Homo sapiens
ttcttcaggttggctcacgtctgccctctcctaggatcatgggactctcccgtgatcccacagagaaaacagccagaatccaccacctatgcacctccacggaggtctccttctccaccaagccacagggacttgctgctatgcaatggtggcattcattgtgatgccagacagagctgacag

SEQ ID NO: 250 - DNA, 968 bp, Homo sapiens; identical to SEQ ID NO: 92

SEQ ID NO: 251 - DNA, 401 bp, Homo sapiens; identical to SEQ ID NO: 93

SEQ ID NO: 252 - DNA, 393 bp, Homo sapiens; identical to SEQ ID NO: 9

SEQ ID NO: 253 - DNA, 453 bp, Homo sapiens; identical to SEQ ID NO: 10

SEQ ID NO: 254 - DNA, 737 bp, Homo sapiens
ctcaaactcccagaccgaaataatcctccttccctggcttcccaaagtgttgtgattatagccgtgagccactgagtctggcatatcttttctcattatgagcgacattccacctcactgagtctggcgtatcttttcttggtatcagcgacattccaccttcgctctattaattattttgagatgtacaataaatcattattaagtgtagtcatcctgtgccactgaacactagatattattccttctaagcaagtataatttaacccacccccatcccctctttgatccctcgcttaccagttcacattacttgtatcaaaatatcacatgtatgccaaaaatacctacaacttttacgtacaaattttttaaataagtaaaaattaataaaaaagggtatctccaacaaagtgataaaataggaggctctaatttgttcctccatccacaaatgcaacaaataaagagccacacccacatcaattccctatgagataaacttacaaacaagttgggatactcttgcatgtagggttatgaaaatacttacttaaaaaaaggtaagaaaaactgaatcatgatctttttctagcgtttatctctgacacattgccctagaatcaatagggaactgttatttcacagcttctctcagagaactgaagtattaatccacatatgtaaagccccagctgttaacagctgcttctcaatgaaatgattcctcacttgcc

SEQ ID NO: 255 - DNA, 601 bp, Homo sapiens; identical to SEQ ID NO: 12

SEQ ID NO: 256 - DNA, 601 bp, Homo sapiens; identical to SEQ ID NO: 11

SEQ ID NO: 257 - DNA, 452 bp, Homo sapiens
aaaggcagaactgccaggtctgtgtcttattttctttgtcattctaatttatcttttttttttttttttttttttttttttttttttttttttttttttttgagacggagtctcactctgtcgcccaggctggagtgcagtggcgggatctcggctcactgcaagctccgcctcccgggttcacgccattctcctgcctcagcctcccaagtagctgggactacaggcgcccgccgctacgcccggctaattttttgtatttttagtagagacggggtttccccgttttagccgggatggtctcgatctcctgacctcgtgatccgcccgcctcggcctcccaaagtgctgggattacaggcgtgagccaccgcgcccggcctctaatttatcttttggtatggagagcagaaaacagtctgaggatgtgacagccctcctcctagagttgg

SEQ ID NO: 258 - DNA, 484 bp, Homo sapiens
gacaacaccaccgtactccagcctagctgacagaacgagacctgtctcttaaaaacggaacaaaacaaaataaaacttgttgactgactaggtattggaaataacaaaaaaggtttccctccacttccttcctttttttaaaaattatgtatattatctctgtgcctggtttttctttattccatgattttcctttgactgtattctcttctttagtcctttcagttccttgtacttgctaatgcaggctgaaatgctagctagaggtacataaagtttttgccaaaagggttttgaaattgtggacttttttttttttcagtcaatgccacgtaagtcacagaaaaaaaaaaaatccttcatgaggaaaaaagaaacatttcacccgctcctttttgtggtgatcagagattgattaatttagttttgcattaatcaagttgtcatcagaaccttccctgctaatctgtaaatgctgcccctg

SEQ ID NO: 259 - DNA, 423 bp, Homo sapiens
ggtctgctgcaccttcattttagccaatttcacccatttggaatgggtgtatttactcaatgcctgtaccctcattgtatctaggaagttactaacttacttttgattttacaggctcatacgtgagacttgccttgtctcaggtgagactttgaacttggacttttgggtgaaatgttggaacctggacccaatgctagctagaggtacataaagtttttgccaaaagggttttgaaattgtggactttttttttttcagtcaatgccacgtaagtcacagaaaaaaaaaaaatccttcatgaggaaaaaagaaacatttcacccgctcctttttgtggtgatcagagattgattaatttagttttgcattaatcaagttgtcatcagaaccttccctgctaatctgtaaatgctgcccctg

SEQ ID NO: 260 - DNA, 527 bp, Homo sapiens; identical to SEQ ID NO: 15

SEQ ID NO: 261 - DNA, 755 bp, Homo sapiens; identical to SEQ ID NO: 16

SEQ ID NO: 262 - DNA, 801 bp, Homo sapiens; identical to SEQ ID NO: 100

SEQ ID NO: 263 - DNA, 340 bp, Homo sapiens; identical to SEQ ID NO: 101

SEQ ID NO: 264 - DNA, 996 bp, Homo sapiens
tgcctgcctctgtagactctgcctgtgggggcagggcatagccaaacaaaaggcagcagaaacctatgcagacttaaatatctctgtctgacagctttgaagaaaatagtggttctcccagcacgcagcttgaaatctcagaacagacagactgcctcttcaagtgggtccctgaaccccacatagcctaactgggaggcaccccccagtagctgcagactgacacctcacatggctgggtactcctctgaggcaaaacttccagaggaacaatcagccagcaacatttgctgttcaccaatatctgctgttctgcaggctccgccgctgatacccagggaaacagggtctggagtggacctccagcaaactccaacagacctgcagctgagggtcctgactgttagaaggaaaactaacaaacagaaaggacatccacaccaaaaccccatctgtatgtcaccgtcatcaaagaccaaaggtagataaaaccacaaagattgggaaaaaacagagcagaaaaactggaaactctaaaaatcagaacacctctcctcctccaaatgaacgcagctccttaccagcagtggaacaaagctggatggagaatgactttgacgtgttgaaagaagaaggtttcagatgatcaaactactccgagctaaaggaggaagttcgaacccatggcaaaggagctgaaaaccttgaaaaaaaaaattagatgaatggctaactagaataaccaaagcagagaagcccttaaaggacctgatggagctgaaaaccatggcacgagaactacgtgatgaacgcacaagcctcattagccgatttgatcaactggaagaaagggtatcagtgatgtaagattaaatgaatgaaatgaagtgagaagagaagttaagagaaaaaagaataaaaagaaatgaacaaagcctccaaaaaatttgggactatgtgaaaagaccaaagtgacatctgattggtatacctgaa

SEQ ID NO: 265 - DNA, 721 bp, Homo sapiens
tgtgccacagaaaaccaattttaaaaaacatactctcagctctacttagtaaaaaatgaaaaaattaatggtcatactgcccaaggtaatttatagattcaatgccatccccatcaagctaccaaagactttcttcactgaattggaaaaaactattttaaagttcatatagaaccaaaaaagagcccacatcaccaagtcaatcctaagccaaaagaacaaagctggaggcatcacgctacctgacttcaaactatactacaaggctacagtaaccaaaacagcatggtactggtaccaaaacagagatatagattaatggaacagaaaataatgccgcatatctacaactatctgatctttgacaaacctgagaaaaacaagcaatggggaaaggattccctacttaataaatggtgcggggaaaactggctagccataggtagaaagctgaaactggatcccttccttacgccttacacaaaaattaattcaagatgtattaaagacttaagcgttagacctgaaaccataaaaacccgagaagaaaacctaggcattaccatccaggacataggcatgggcaaggacttcatgtctaaaacaccaaaagcaatggcaacaaaagccaaaattgacaaatgggatctaattaaactaaagagcttctgcacagcaaaagaaactaccatcagagtgaacaagcaacctacaaaatggg

SEQ ID NO: 266 - DNA, 256 bp, Homo sapiens
tgagcttagacccggaaagctctggatgcaaggctcagtcctctcttctgcagaaaagagtcatgacctaatactctagccagctgcccagagccttctgtaatcctaaactggctagccataggtagaaagctgaaactggatcccttccttacgccttacacaaaaattaattcaagatgtattaaagacttaagcgttagacctgaaaccataaaaacccgagaagaaaacctaggcattaccatccaggaca

SEQ ID NO: 267 - DNA, 410 bp, Homo sapiens
aacaatcagccagcaacatttgctgttcaccaatatctgctgttctgcaggctccgccgctgatacccagggaaacagggtctggagtggacctccagcaaactccaacagacctgcagctgagggtcctgactgttagaaggaaaactaacaaacagaaaggacatccacaccaaaaccccatctgtatgtcaccgtcatcaaagaccaaaggtagataaaaccacaaagattgggaaaaaacagagcagaaaaactggaaactctaaaaatcagagcaagcactatgggaggccatggcaggtggatcacctgaggacaggagtttgagaccagcctggccaacatggtgaaattctgtctctactaaaagtacaaaaattagccgggcgtggtggtaggcacatgta

SEQ ID NO: 268 - DNA, 601 bp, Homo sapiens; identical to SEQ ID NO: 104

SEQ ID NO: 269 - DNA, 677 bp, Homo sapiens; identical to SEQ ID NO: 105

SEQ ID NO: 270 - DNA, 507 bp, Homo sapiens
attccactccattccactgcgctccattcctctccactccatcgtattccattttttcccattccattccattccactcctttccactctgctccactccactcctctccattcaattctattccaccccattcctttccactccattctactccactccacttcactgtattccatttgatttcattcaatgccattcgaatccatttgattcgattccattcgattccatttgatttcatttttatgtatggtagatatacataaaatatatatgtttgaatatacatcaatttctatgtaataaacatgtatatcgccgttatgttttgccagacagtgatcaaagcactttcttcccaaagatagatgaattctactgttctcccaaatatttgttagcttctaaaatatacacattttgctatcatatataccaggttggtgggcaggtgtggtgtttcatgcctgtaatcccaggactttggaagggaggccatgacatta

SEQ ID NO: 271 - DNA, 169 bp, Homo sapiens; identical to SEQ ID NO: 106

SEQ ID NO: 272 - DNA, 461 bp, Homo sapiens; identical to SEQ ID NO: 17

SEQ ID NO: 273 - DNA, 645 bp, Homo sapiens; identical to SEQ ID NO: 18

SEQ ID NO: 274 - DNA, 20 bp, Artificial Sequence
  probe for STS
gaactctgcaaacctcctgg

SEQ ID NO: 275 - DNA, 18 bp, Artificial Sequence
  probe for STS
ttttgaggcggagtctcg

SEQ ID NO: 276 - DNA, 20 bp, Artificial Sequence
  probe for STS
gtgggtgcatgatgtactgc

SEQ ID NO: 277 - DNA, 20 bp, Artificial Sequence
  probe for STS
ccgttaaccaacgagtggat

SEQ ID NO: 278 - DNA, 21 bp, Artificial Sequence
  probe for STS
gggtcctagataggctccaag

SEQ ID NO: 279 - DNA, 20 bp, Artificial Sequence
  probe for STS
ttcatgttggcagtgattgg

SEQ ID NO: 280 - DNA, 20 bp, Artificial Sequence
  probe for STS
aggctactgtgaatcacgcc

SEQ ID NO: 281 - DNA, 20 bp, Artificial Sequence
  probe for STS
gtgcatgtgttccctttgtg

SEQ ID NO: 282 - DNA, 20 bp, Artificial Sequence
  probe for STS
cgtcggtattttacgacacg

SEQ ID NO: 283 - DNA, 20 bp, Artificial Sequence
  probe for STS
gcatttgtttttcatgtgcg

SEQ ID NO: 284 - DNA, 20 bp, Artificial Sequence
  probe for STS
acctccgaagactgaaccag

SEQ ID NO: 285 - DNA, 20 bp, Artificial Sequence
  probe for STS
cccttgagtccacagagtcc

SEQ ID NO: 286 - DNA, 20 bp, Artificial Sequence
  probe for STS
ccagacgttctaccctttcg

SEQ ID NO: 287 - DNA, 20 bp, Artificial Sequence
  probe for STS
ctcccttggttcatgccatt

SEQ ID NO: 288 - DNA, 20 bp, Artificial Sequence
  probe for STS
tcccctcagccatctgtatt

SEQ ID NO: 289 - DNA, 20 bp, Artificial Sequence
  probe for STS
ggcccacctgagtaatggta

SEQ ID NO: 290 - DNA, 20 bp, Artificial Sequence
  probe for STS
tttttctaaccttgcctgcg

SEQ ID NO: 291 - DNA, 20 bp, Artificial Sequence
  probe for STS
tgcagagaagcagcctacaa

SEQ ID NO: 292 - DNA, 20 bp, Artificial Sequence
  probe for STS
atctgcacacttgggtaggc

SEQ ID NO: 293 - DNA, 20 bp, Artificial Sequence
  probe for STS
gaggaaatgcagaatttggg

SEQ ID NO: 294 - DNA, 20 bp, Artificial Sequence
  probe for STS
gctacttgtgaatcacgcca

SEQ ID NO: 295 - DNA, 21 bp, Artificial Sequence
  probe for STS
tgcatatttcgaagcattgtc

SEQ ID NO: 296 - DNA, 20 bp, Artificial Sequence
  probe for STS
tgctcctccttctcacgttt

SEQ ID NO: 297 - DNA, 20 bp, Artificial Sequence
  probe for STS
tcccgtacatgtcttgttgc

SEQ ID NO: 298 - DNA, 20 bp, Artificial Sequence
  probe for STS
ccatcctcacgacttggact

SEQ ID NO: 299 - DNA, 20 bp, Artificial Sequence
  probe for STS
aaggcagatttttgaatggg

SEQ ID NO: 300 - DNA, 20 bp, Artificial Sequence
  probe for STS
tggaataccctggattggaa

SEQ ID NO: 301 - DNA, 20 bp, Artificial Sequence
  probe for STS
cttatgcctcaaatcgctcc

SEQ ID NO: 302 - DNA, 20 bp, Artificial Sequence
  probe for STS
gactggagtggaacggtctc

SEQ ID NO: 303 - DNA, 20 bp, Artificial Sequence
  probe for STS
tcacttccctccgattttct

SEQ ID NO: 304 - DNA, 24 bp, Artificial Sequence
  probe for STS
ttgactgcttattctacacaaggc

SEQ ID NO: 305 - DNA, 18 bp, Artificial Sequence
  probe for STS
caggggaaattgggtttt

SEQ ID NO: 306 - DNA, 20 bp, Artificial Sequence
  probe for STS
gggtcggtgcacctaaataa

SEQ ID NO: 307 - DNA, 27 bp, Artificial Sequence
  probe for STS
tgcactaaagagtgataataaattctg

SEQ ID NO: 308 - DNA, 20 bp, Artificial Sequence
  probe for STS
tcaggtactgtgatgccgtt

SEQ ID NO: 309 - DNA, 21 bp, Artificial Sequence
  probe for STS
tgaagaggacacaaagggaaa

SEQ ID NO: 310 - DNA, 20 bp, Artificial Sequence
  probe for STS
tcacagctcagttctttggg

SEQ ID NO: 311 - DNA, 20 bp, Artificial Sequence
  probe for STS
tgtccatgttggatgaggaa

SEQ ID NO: 312 - DNA, 20 bp, Artificial Sequence
  probe for STS
cctttttgcaactgtggaga

SEQ ID NO: 313 - DNA, 20 bp, Artificial Sequence
  probe for STS
agctgggtattccatcgtga

SEQ ID NO: 314 - DNA, 20 bp, Artificial Sequence
  probe for STS
atctagaattgggccttgcc

SEQ ID NO: 315 - DNA, 18 bp, Artificial Sequence
  probe for STS
tggggaacgttgcctaac

SEQ ID NO: 316 - DNA, 21 bp, Artificial Sequence
  probe for STS
tgaaatgtaaaaccgtggaca

SEQ ID NO: 317 - DNA, 18 bp, Artificial Sequence
  probe for STS
tggggaacgttgcctaac

SEQ ID NO: 318 - DNA, 19 bp, Artificial Sequence
  probe for STS
agggtcccctccctacaat

SEQ ID NO: 319 - DNA, 20 bp, Artificial Sequence
  probe for STS
gagacccctgcttgaacaat

SEQ ID NO: 320 - DNA, 20 bp, Artificial Sequence
  probe for STS
ggccccgtaggtctatacaa

SEQ ID NO: 321 - DNA, 20 bp, Artificial Sequence
  probe for STS
ggtcatgacttgccatgttg

SEQ ID NO: 322 - DNA, 20 bp, Artificial Sequence
  probe for STS
tccaagcatatagttgcccc

SEQ ID NO: 323 - DNA, 20 bp, Artificial Sequence
  probe for STS
tctttcactgcacatggagc

SEQ ID NO: 324 - DNA, 21 bp, Artificial Sequence
  probe for STS
agaaaacttgcaatgcaggag

SEQ ID NO: 325 - DNA, 20 bp, Artificial Sequence
  probe for STS
actgcaccaacaaatgctca

SEQ ID NO: 326 - DNA, 21 bp, Artificial Sequence
  probe for STS
gctccacattagaatgaccca

SEQ ID NO: 327 - DNA, 21 bp, Artificial Sequence
  probe for STS
ggaaaaatattgtgaccccca

SEQ ID NO: 328 - DNA, 21 bp, Artificial Sequence
  probe for STS
ttgcaacattgcacttgtagg

SEQ ID NO: 329 - DNA, 20 bp, Artificial Sequence
  probe for STS
atgattctggtcttggtgcc

SEQ ID NO: 330 - DNA, 20 bp, Artificial Sequence
  probe for STS
cctatctctgcatggcctgt

SEQ ID NO: 331 - DNA, 20 bp, Artificial Sequence
  probe for STS
aaggcaagttttggagcaga

SEQ ID NO: 332 - DNA, 20 bp, Artificial Sequence
  probe for STS
atctcacatggtgccagaca

SEQ ID NO: 333 - DNA, 20 bp, Artificial Sequence
  probe for STS
aaggcaagttttggagcaga

SEQ ID NO: 334 - DNA, 20 bp, Artificial Sequence
  probe for STS
tgcacttgtaggaatttgga

SEQ ID NO: 335 - DNA, 27 bp, Artificial Sequence
  probe for STS
aaaacagacatatatacacatacactg

SEQ ID NO: 336 - DNA, 20 bp, Artificial Sequence
  probe for STS
ggcttaaacttgggagggtg

SEQ ID NO: 337 - DNA, 20 bp, Artificial Sequence
  probe for STS
gcacttcaaaccgaggctta

SEQ ID NO: 338 - DNA, 22 bp, Artificial Sequence
  probe for STS
gcaaagttcttgcactgtgttt

SEQ ID NO: 339 - DNA, 20 bp, Artificial Sequence
  probe for STS
ttgtggcctgtggtgtagaa

SEQ ID NO: 340 - DNA, 22 bp, Artificial Sequence
  probe for STS
gcaaagttcttgcactgtgttt

SEQ ID NO: 341 - DNA, 20 bp, Artificial Sequence
  probe for STS
aaaggaaaccttgagcagga

SEQ ID NO: 342 - DNA, 23 bp, Artificial Sequence
  probe for STS
ggttttgtattcgtttgctctca

SEQ ID NO: 343 - DNA, 20 bp, Artificial Sequence
  probe for STS
tgcaatgaaatgagaaccca

SEQ ID NO: 344 - DNA, 20 bp, Artificial Sequence
  probe for STS
gtcaggaggcttaggaaggc

SEQ ID NO: 345 - DNA, 20 bp, Artificial Sequence
  probe for STS
ctgggattactggcaaggac

SEQ ID NO: 346 - DNA, 20 bp, Artificial Sequence
  probe for STS
gtgctgttaagcaccatcca

SEQ ID NO: 347 - DNA, 20 bp, Artificial Sequence
  probe for STS
ctgggattactggcaaggac

SEQ ID NO: 348 - DNA, 21 bp, Artificial Sequence
  probe for STS
gcaacatagatggacccagaa

SEQ ID NO: 349 - DNA, 20 bp, Artificial Sequence
  probe for STS
atagcaaagagcctcccaga

SEQ ID NO: 350 - DNA, 20 bp, Artificial Sequence
  probe for STS
ccatctcctgacctcgtgat

SEQ ID NO: 351 - DNA, 20 bp, Artificial Sequence
  probe for STS
tcatttttgcagtgagcgag

SEQ ID NO: 352 - DNA, 20 bp, Artificial Sequence
  probe for STS
ctggcaggttcatggtcttt

SEQ ID NO: 353 - DNA, 19 bp, Artificial Sequence
  probe for STS
agcccaacatcaccaacca

SEQ ID NO: 354 - DNA, 20 bp, Artificial Sequence
  probe for STS
tcgggttcaacagattctcc

SEQ ID NO: 355 - DNA, 20 bp, Artificial Sequence
  probe for STS
ttttctacaggccacagcaa

SEQ ID NO: 356 - DNA, 21 bp, Artificial Sequence
  probe for STS
caatggcattcactgtgaaca

SEQ ID NO: 357 - DNA, 20 bp, Artificial Sequence
  probe for STS
cttgaaactcctccagctgg

SEQ ID NO: 358 - DNA, 22 bp, Artificial Sequence
  probe for STS
catgcagaagatcatccacgta

SEQ ID NO: 359 - DNA, 24 bp, Artificial Sequence
  probe for STS
cagacaagttattgaaggtagcca

SEQ ID NO: 360 - DNA, 20 bp, Artificial Sequence
  probe for STS
gcactgaaggtgtttcacga

SEQ ID NO: 361 - DNA, 20 bp, Artificial Sequence
  probe for STS
tctcaaatgccagagagcct

SEQ ID NO: 362 - DNA, 19 bp, Artificial Sequence
  probe for STS
ttcttcaggttggctcacg

SEQ ID NO: 363 - DNA, 20 bp, Artificial Sequence
  probe for STS
ctgtcagctctgtctggcat

SEQ ID NO: 364 - DNA, 24 bp, Artificial Sequence
  probe for STS
aaccccatctctagcaaaaatatg

SEQ ID NO: 365 - DNA, 20 bp, Artificial Sequence
  probe for STS
taggtgacagggcaggattc

SEQ ID NO: 366 - DNA, 20 bp, Artificial Sequence
  probe for STS
ggtgtcccatggaaacctta

SEQ ID NO: 367 - DNA, 20 bp, Artificial Sequence
  probe for STS
ttacaggcagcatttttccc

SEQ ID NO: 368 - DNA, 19 bp, Artificial Sequence
  probe for STS
gttggcaacttgcactgct

SEQ ID NO: 369 - DNA, 20 bp, Artificial Sequence
  probe for STS
cctttcctctcaaagtcccc

SEQ ID NO: 370 - DNA, 22 bp, Artificial Sequence
  probe for STS
tcatttgtgtccttctcttgga

SEQ ID NO: 371 - DNA, 20 bp, Artificial Sequence
  probe for STS
ctaagccaggaacttgccac

SEQ ID NO: 372 - DNA, 20 bp, Artificial Sequence
  probe for STS
ctcaaactcccagaccgaaa

SEQ ID NO: 373 - DNA, 21 bp, Artificial Sequence
  probe for STS
ggcaagtgaggaatcatttca

SEQ ID NO: 374 - DNA, 20 bp, Artificial Sequence
  probe for STS
actaccatttctggaagccg

SEQ ID NO: 375 - DNA, 20 bp, Artificial Sequence
  probe for STS
ctcccttggttcatgccatt

SEQ ID NO: 376 - DNA, 20 bp, Artificial Sequence
  probe for STS
ccagacgttctaccctttcg

SEQ ID NO: 377 - DNA, 20 bp, Artificial Sequence
  probe for STS
gagccgagatccagttacca

SEQ ID NO: 378 - DNA, 18 bp, Artificial Sequence
  probe for STS
aaaggcagaactgccagg

SEQ ID NO: 379 - DNA, 20 bp, Artificial Sequence
  probe for STS
ccaactctaggaggagggct

SEQ ID NO: 380 - DNA, 20 bp, Artificial Sequence
  probe for STS
gacaacaccaccgtactcca

SEQ ID NO: 381 - DNA, 20 bp, Artificial Sequence
  probe for STS
caggggcagcatttacagat

SEQ ID NO: 382 - DNA, 20 bp, Artificial Sequence
  probe for STS
ggtctgctgcaccttcattt

SEQ ID NO: 383 - DNA, 20 bp, Artificial Sequence
  probe for STS
caggggcagcatttacagat

SEQ ID NO: 384 - DNA, 20 bp, Artificial Sequence
  probe for STS
taaaaggcagaactgccagg

SEQ ID NO: 385 - DNA, 20 bp, Artificial Sequence
  probe for STS
gggagaaaagttctgcaacg

SEQ ID NO: 386 - DNA, 18 bp, Artificial Sequence
  probe for STS
tgggcgaggactttatga

SEQ ID NO: 387 - DNA, 20 bp, Artificial Sequence
  probe for STS
ggggtcccagttccactatt

SEQ ID NO: 388 - DNA, 20 bp, Artificial Sequence
  probe for STS
gtgggggtttcacattatgg

SEQ ID NO: 389 - DNA, 23 bp, Artificial Sequence
  probe for STS
ggtcacagactcacatttaagca

SEQ ID NO: 390 - DNA, 20 bp, Artificial Sequence
  probe for STS
acctaagggaacccaggaga

SEQ ID NO: 391 - DNA, 21 bp, Artificial Sequence
  probe for STS
cgacacttttgggaagtttca

SEQ ID NO: 392 - DNA, 21 bp, Artificial Sequence
  probe for STS
tgcctgcctctgtagactctg

SEQ ID NO: 393 - DNA, 25 bp, Artificial Sequence
  probe for STS
ttcaggtataccaatcagatgtcac

SEQ ID NO: 394 - DNA, 18 bp, Artificial Sequence
  probe for STS
tgtgccacagaaaaccaa

SEQ ID NO: 395 - DNA, 20 bp, Artificial Sequence
  probe for STS
cccattttgtaggttgcttg

SEQ ID NO: 396 - DNA, 19 bp, Artificial Sequence
  probe for STS
tgagcttagacccggaaag

SEQ ID NO: 397 - DNA, 19 bp, Artificial Sequence
  probe for STS
tgtcctggatggtaatgcc

SEQ ID NO: 398 - DNA, 20 bp, Artificial Sequence
  probe for STS
aacaatcagccagcaacatt

SEQ ID NO: 399 - DNA, 20 bp, Artificial Sequence
  probe for STS
tacatgtgcctaccaccacg

SEQ ID NO: 400 - DNA, 20 bp, Artificial Sequence
  probe for STS
attgatctccttggttcccc

SEQ ID NO: 401 - DNA, 20 bp, Artificial Sequence
  probe for STS
gacatgtgtggccaatttga

SEQ ID NO: 402 - DNA, 18 bp, Artificial Sequence
  probe for STS
ccgacttccacaatggct

SEQ ID NO: 403 - DNA, 20 bp, Artificial Sequence
  probe for STS
gggagaaaagttctgcaacg

SEQ ID NO: 404 - DNA, 20 bp, Artificial Sequence
  probe for STS
attccactccattccactgc

SEQ ID NO: 405 - DNA, 20 bp, Artificial Sequence
  probe for STS
taatgtcatggcctcccttc

SEQ ID NO: 406 - DNA, 20 bp, Artificial Sequence
  probe for STS
atcgttttgaatggcgtcaa

SEQ ID NO: 407 - DNA, 19 bp, Artificial Sequence
  probe for STS
ctgcaccactccaatccaa

SEQ ID NO: 408 - DNA, 20 bp, Artificial Sequence
  probe for STS
agtcggagtcggagtgtgat

SEQ ID NO: 409 - DNA, 20 bp, Artificial Sequence
  probe for STS
attccattgctttccattgc

SEQ ID NO: 410 - DNA, 20 bp, Artificial Sequence
  probe for STS
gagctgcaacataacaggca

SEQ ID NO: 411 - DNA, 20 bp, Artificial Sequence
  probe for STS
aggggaacatcacactctgg

SEQ ID NO: 412 - DNA, 495 bp, Homo sapiens
tgtgtgtgcactttctcaaaacactttattcatatggtacttcttctcttcaaatataaaaatcagattgtaagttctttttttttttttaaagatccaggtttaaaaatgtctactttttgaccaaccttaccccatggttttaaatttttaattgttaatatttgaaaagtgttacaatgctggtctctttcaccctcttagaataatcatgtacatggacagccatatacaggaccagcagcacatcacttgaacaaccctcagaaaacaggccaacgaacacaagaaaattatgaaggcaatgaagaagtatcctcacctcagatgaaggatcagtgaaaagcaataattaactgcttcctttatgactatgcactaaggtcttatagtccaaactttctctgtgtctggctagtattgaaaactagataaactgctccaaaccaacatggagtaaagagcatattcactggtttatttgcagtaatttgc

SEQ ID NO: 413 - DNA, 22 bp, Artificial Sequence
  probe for STS
tgtgtgtgcactttctcaaaac

SEQ ID NO: 414 - DNA, 23 bp, Artificial Sequence
  probe for STS
gcaaattactgcaaataaaccag

(19) United States
(12) Patent Application Publication
(10) Pub. No.: US 2006/0166228 A1
(43) Pub. Date: Jul. 27, 2006

(54) Markers of alterations in the Y chromosome and uses therefor

(76) Inventors: David C. Page, Winchester, MA (US); Steven G. Rozen, Concord, MA (US); Helen Skaletsky, Milton, MA (US); Sjoerd Repping, Amsterdam (NL); Tomoko Kuroda-Kawaguchi, Musashino City (JP); Julian Lange, Boston, MA (US); Laura Brown, East Sandwich, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 11/195,344

(22) Filed: Aug. 1, 2005

Related U.S. Application Data
(60) Provisional application No. 60/592,719, filed on Jul. 30, 2004.

(51) Int. Cl. C12Q 1/68; C12M 1/34

(52) U.S. Cl. 435/6; 435/287.2

(57) ABSTRACT

Novel sequence tagged sites (STSs), probes and primers useful, e.g., for detecting the presence or absence of an STS in a sample, and methods of using these STSs, probes and primers, e.g., in methods of detecting alterations in the Y chromosome are disclosed. These compositions are also useful in methods of diagnosing or aiding in the diagnosis and/or cause of reduced sperm count and in methods of predicting or aiding in the prediction of the likelihood of success of infertility treatments.

### RELATED APPLICATIONS

This application claims the benefit of U.S. Provisional Application Ser. No. 60/592,719 filed Jul. 30, 2004, the entire disclosure of which is incorporated herein by reference.

### GOVERNMENT SUPPORT

Work described herein was funded, in whole or in part, by grants from the National Institutes of Health (Grant Nos. NICHD-HD32907 and NHGRI-HG00257). The United States Government has certain rights in the invention.

### BACKGROUND OF THE INVENTION

At least one in every ten couples of reproductive age is unable to bear children despite an extended period of unprotected sexual intercourse. In recent years, there has been an intensive search for genetic causes of infertility in both men and women. Spermatogenic failure is the most common form of male infertility, and here the most striking genetic findings have emerged from studies of the Y chromosome's long arm (Yq). It is now widely accepted that deletions in any one of three Yq regions—AZFa, AZFb, or AZFc—can severely diminish or extinguish sperm production. The number and type of Y chromosomal deletions in a male can have widely varying effects on the success of infertility treatments that a couple may choose to undergo. However, despite the region's biological and medical importance, efforts to develop physical maps have been stymied by the region's unusually repetitive sequence composition, and past studies have suggested that it would be difficult or impossible to identify single-copy DNA markers, localize deletion breakpoints, and accurately identify alterations of the Y chromosome.

### SUMMARY OF THE INVENTION

The invention pertains in part to novel sequence tagged sites (STSs), to probes and primers useful, e.g., for detecting the presence or absence of an STS in a sample, and to methods of using these STSs, probes and primers, e.g., in methods of detecting alterations in the Y chromosome. These compositions are also useful in methods of diagnosing or aiding in the diagnosis and/or cause of reduced sperm count (oligospermia or azospermia) and in methods of predicting or aiding in the prediction of the likelihood of success of infertility treatments.

Described herein are results of the assessment and characterization of the human Y chromosome, particularly the AZFc region of the human Y chromosome. As a result of this work, important sequence landmarks of the Y chromosome, particularly AZFc, have been identified. In particular, STSs that can be used in evaluating Y chromosomal DNA for alterations, e.g., deletions such as microdeletions, have been identified; these alterations may be associated with reduced sperm count (e.g., azoospermia and/or oligospermia). The identified STSs and probes and primers therefor can be used in methods of analyzing Y chromosomal DNA for such alterations and for determining or confirming that a deletion or set of deletions is linked to (indicative of) reduced sperm count (azoospermia or severe oligospermia) in humans.

Accordingly, in some embodiments the invention pertains to a method of detecting an alteration in the human Y chromosome comprising assessing a nucleic acid sample from an individual to be tested for the presence or absence of one or more nucleic acid molecules comprising a nucleic acid sequence selected from the group consisting of SEQ ID NOS: 1-20, 61-108, 205-273 and 412, wherein the absence of one or more of said nucleic acid sequences is indicative of an alteration in the human Y chromosome in the individual. In one embodiment the AZFc region of the Y chromosome is altered. In a particular embodiment the alteration is a deletion in the Y chromosome, e.g., a deletion selected from the group consisting of the deletions shown in FIGS. 2, 3A-3B, 4A-4B and 8.

In some embodiments the nucleic acid sample is a is a genomic DNA sample. In particular embodiments the sample is derived from blood, skin, sperm, hair root, saliva or buccal cells, or from cells cultured from blood or skin. In other embodiments the individual to be tested is a male with reduced sperm count.

In a particular method of the invention, the presence or absence of said one or more nucleic acid molecules is determined using one or more probes complementary to the nucleic acid sequence. For example, said one or more probes can be immobilized on a solid support, such as a microarray.

In another method of the invention, the presence or absence of said one or more nucleic acid molecules is determined by amplification using one or more primers complementary to the nucleic acid sequence. For example, the primers selected from the group consisting of SEQ ID NOS: 21-60 can be used to determine the presence or absence of a nucleic acid molecule comprising a nucleic acid sequence selected from the group consisting of SEQ ID NOS: 1-20, and the primers selected from the group consisting of SEQ ID NOS: 109-204 can be used to determine the presence or absence of a nucleic acid molecule comprising a nucleic acid sequence selected from the group consisting of SEQ ID NOS: 61-108. Similarly, primers selected from the group consisting of SEQ ID NOS: 274-411 can be used to determine the presence or absence of a nucleic acid molecule comprising a nucleic acid sequence selected from the group consisting of SEQ ID NOS: 205-273, and the primers selected from the group consisting of SEQ ID NOS: 413-414 can be used to determine the presence or absence of a nucleic acid molecule comprising a nucleic acid sequence of SEQ ID NO: 412.

FIGS. 5A-5F, 6A-6K and 7A-7P show the relationship between the primers of SEQ ID NOS 21-60 and 413-414 and the STSs of SEQ ID NOS: 1-20 and 412, respectively, the primers of SEQ ID NOS: 109-204 and the STSs of SEQ ID NOS: 61-108, and the primers of SEQ ID NOS: 274-411 and the STSs of SEQ ID NOS: 205-273, respectively. As used herein, a primer “corresponds” to an STS if it serves as a specific primer for that STS in an amplification reaction. For example, SEQ ID NOS: 21 and 22 are primers which serve as specific primers for SEQ ID NO: 1, and thus SEQ ID NOS: 21 and 22 are primers which correspond to the STS of SEQ ID NO: 1.

The invention also pertains to a method of predicting or aiding in the prediction of the likelihood of success of an infertility treatment of a male having reduced sperm count, comprising assessing a nucleic acid sample from said male for the presence or absence of one or more nucleic acid molecules comprising a nucleic acid sequence selected from the group consisting of SEQ ID NOS: 1-20, 61-108, 205-273 and 412, wherein the absence of one or more of said nucleic acid sequences is indicative of an alteration in the human Y chromosome in the individual, and determining the likelihood of success of a fertility treatment in view of the type of alteration present, if any.

In one embodiment the AZFc region of the Y chromosome is altered. In a particular embodiment the alteration is a deletion in the Y chromosome, e.g., a deletion selected from the group consisting of the deletions shown in FIGS. 2, 3A-3B, 4A-4B and 8.

In some embodiments the nucleic acid sample is a genomic DNA sample. In particular embodiments the sample is derived from blood, skin, sperm, hair root, saliva or buccal cells, or from cells cultured from blood or skin. In other embodiments the individual to be tested is a male with reduced sperm count. In a particular method of the invention, the presence or absence of said one or more nucleic acid molecules is determined using one or more probes complementary to the nucleic acid sequence. For example, said one or more probes can be immobilized on a solid support, such as a microarray.

In another method of the invention, the presence or absence of said one or more nucleic acid molecules is determined by amplification using one or more primers complementary to the nucleic acid sequence. For example, the primers selected from the group consisting of SEQ ID NOS: 21-60 can be used to determine the presence or absence of a nucleic acid molecule comprising a nucleic acid sequence selected from the group consisting of SEQ ID NOS: 1-20, and the primers selected from the group consisting of SEQ ID NOS: 109-204 can be used to determine the presence or absence of a nucleic acid molecule comprising a nucleic acid sequence selected from the group consisting of SEQ ID NOS: 61-108. Similarly, primers selected from the group consisting of SEQ ID NOS: 274-411 can be used to determine the presence or absence of a nucleic acid molecule comprising a nucleic acid sequence selected from the group consisting of SEQ ID NOS: 205-273, and primers selected from the group consisting of SEQ ID NOS: 413-414 can be used to determine the presence or absence of a nucleic acid molecule comprising a nucleic acid sequence of SEQ ID NO: 412.

The invention also pertains to an isolated nucleic acid molecule comprising a nucleic acid sequence selected from the group consisting of SEQ ID NOS: 21-60, 109-204, 274-411 and 413-414, as well as to an isolated nucleic acid molecule consisting of a nucleic acid sequence selected from the group consisting of SEQ ID NOS: 1-20, 61-108, 205-273 and 412. The invention also pertains to nucleic acid probes capable of specifically hybridizing to a nucleic acid molecule selected from the group consisting of SEQ ID NOS: 1-20, 61-108, 205-273 and 412, and to nucleic acid primers capable of serving as specific primers for amplification of a nucleic acid molecule selected from the group consisting of SEQ ID NOS: 1-20, 61-108, 205-273 and 412.

In another embodiment, the invention relates to a kit comprising one or more isolated nucleic acid molecules capable of serving as a specific primer for amplification of one or more nucleic acid molecules selected from the group consisting of SEQ ID NOS: 1-20, 61-108, 205-273 and 412, amplification reagents, and instructions for using said nucleic acid molecules and reagents to detect the presence or absence of one or more nucleic acid molecules comprising a nucleic acid sequence selected from the group consisting of SEQ ID NOS: 1-20, 61-108, 205-273 and 412. In one embodiment the isolated nucleic acid molecules comprise or consist of a nucleic acid sequence selected from the group consisting of SEQ ID NOS: 21-60, 109-204, 274-411 and 413-414.

In an additional embodiment, the invention relates to a kit comprising one or more isolated nucleic acid molecules capable of serving as a specific probe for one or more nucleic acid molecules selected from the group consisting of SEQ ID NOS: 1-20, 61-108, 205-273 and 412, hybridization reagents, and instructions for using said nucleic acid molecules and reagents to detect the presence or absence of one or more nucleic acid molecules comprising a nucleic acid sequence selected from the group consisting of SEQ ID NOS: 1-20, 61-108, 205-273 and 412. For example, the nucleic acid molecules capable of serving as a specific probes may be selected from the group consisting of SEQ ID NOS: 21-60, 109-204, 274-411 and 413-414.

### BRIEF DESCRIPTION OF THE DRAWINGS

FIGS. 1A-1B are a table listing landmark STSs and their Y chromosomal location.

FIG. 2 is a table showing plus/minus results for STSs distinguishing different types of Y chromosomal deletions. STSs are shown along the top, and deletions are shown down the left side. A minus (“−”) indicates the absence of the indicated STS, while a filled-in square indicates the presence of the indicated STS.

FIGS. 3A-3B are a table showing plus/minus results for a larger set of STSs distinguishing different types of Y chromosomal deletions. A minus (“−”) indicates the absence of the indicated STS, while a filled-in square indicates the presence of the indicated STS.

FIGS. 4A-4B are a table showing plus/minus results for a larger set of STSs distinguishing different types of Y chromosomal deletions. A minus (“−”) indicates the absence of the indicated STS, while a filled-in square indicates the presence of the indicated STS.

FIGS. 5A-5F show the nucleotide sequences of the STSs in FIG. 2 (SEQ ID NOS: 1-20 and 412), as well as the nucleotide sequences of probes and primers which can be used to identify the presence or absence of the corresponding STS (SEQ ID NOS: 21-60 and 413-414).

FIGS. 6A-6K show the nucleotide sequences of the STSs in FIGS. 3A-3B (SEQ ID NOS: 61-108), as well as the nucleotide sequences of probes and primers which can be used to identify the presence or absence of the corresponding STS (SEQ ID NOS: 109-204).

FIGS. 7A-7P show the nucleotide sequences of the STSs in FIGS. 4A-4B (SEQ ID NOS: 205-273), as well as the nucleotide sequences of probes and primers which can be used to identify the presence or absence of the corresponding STS (SEQ ID NOS: 274-411).

FIG. 8 is an abbreviated table showing plus/minus results distinguishing different types of deletions involving AZFc.

FIG. 9 shows a genealogical analysis of SFV patterns associated with b2/b3 and gr/gr deletions. In the SFV patterns, “C” indicates the cut variant described by Fernandes et al. (N. Am J Hum Genet 74:180-187 (2004)), “U” indicates the uncut variant, “B” indicates both variants, and “+” and “−” indicate the presence or absence, respectively, of the Y-DAZ3 variant. The order of SFVs is as shown in table 2 in the work of Fernandes et al. (2004): DAZ-SNV I, DAZ-SNV II, sY586 (DAZ-SNV III), DAZ-SNV IV, sY587 (DAZ-SNV V), DAZ-SNV VI, AZFc SFV 18 (assayed by Y-DAZ3), TTY4-SNV I, BPY2-SNV, GOLY-SNV I, and AZFc SFV 20 (AZFc-P1-SNV I) (Saxena et al., Genomics 67:256-267 (2000); Kuroda-Kawaguchi et al., Nat Genet 29:279-286 (2001); Fernandes et al., Mol Hum Reprod 8:286-298 (2002); Fernandes et al., N. Am J Hum Genet 74:180-187 (2004)). The genealogical tree of extant human Y chromosomes and the branch designations are from the studies by Underhill et al. (Nat Genet 26:358-361 (2000) and the Y-Chromosome Consortium (Genome Res 12:339-348 (2002)).

### DETAILED DESCRIPTION OF THE INVENTION

A description of preferred embodiments of the invention follows.

Sequence tagged sites (STSs) are short sequences for which the exact location in the genome and order of bases are known. Because each is unique, STSs are helpful for chromosome placement of mapping and sequencing data and serve as landmarks on the physical map of the human genome. The primary sequence and presence or absence of alterations in the sequence of the Y chromosome, particularly deletions, are particularly difficult to determine due to the extensive blocks of sequence repeats within this region. As described herein, STSs have been identified which are uniquely suited for use in methods of detecting alterations in the Y chromosome. In particular, these STSs, individually or more preferably in combination, allow the detection of deletions in the Y chromosome which are difficult to detect and/or distinguish from other alterations in the Y chromosome using other markers and methods.

The ability to detect Y chromosomal alterations, e.g., deletions, and to differentiate between different types of alterations has significant implications for infertility treatment regimens. Infertility treatments can be invasive and the procedures, together with the accompanying stress, can impose a significant burden on both partners physiologically, emotionally and financially. In the end, in some instances these procedures do not result in successful pregnancies. The compositions and methods of the invention provide the couple with additional information which can inform their decision on whether to proceed with infertility treatments and which procedures are likely to be effective, based on the alterations, if any, detected in the male's Y chromosome. Many Y chromosomal deletions have known effects on the ability of the male to produce viable sperm. Using the methods and compositions of the invention it can be determined which, if any, of the described deletions are present in the male's Y chromosome and what the effect of the deletion(s) is on the ability to obtain viable sperm from the male. For example, if a p5/p1 Y chromosomal deletion is detected in the male, the likelihood of obtaining viable sperm from him is very low. This information can then guide the couple in determining which, if any, infertility treatments to pursue, rather than proceeding blindly with a course of action unlikely to produce results.

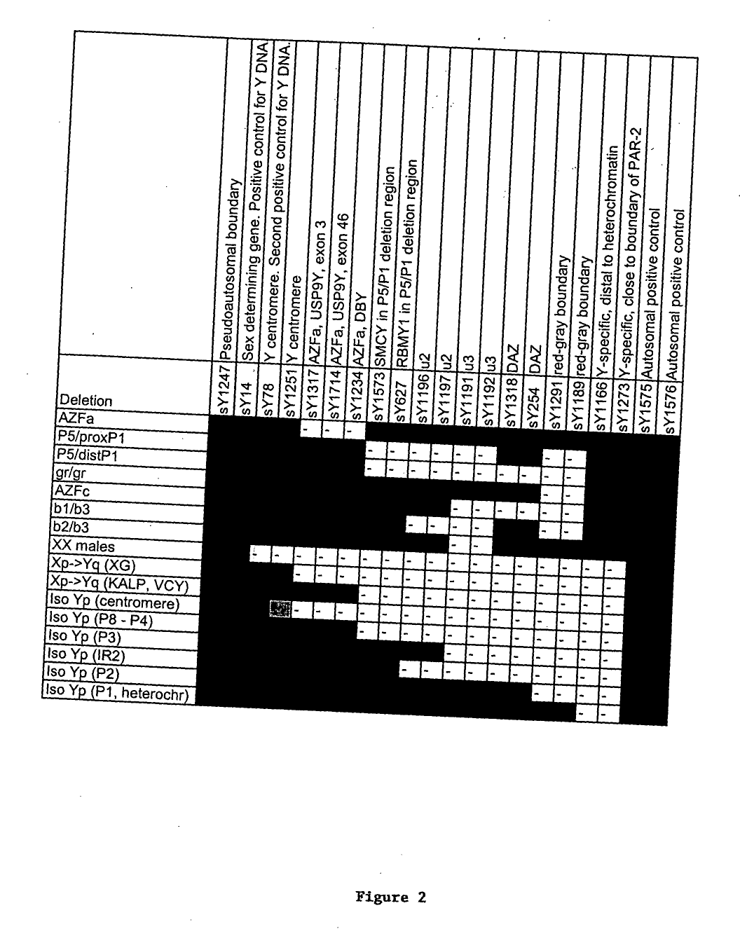

Thus, the invention pertains, in part, to a method of detecting an alteration in the human Y chromosome comprising assessing a nucleic acid sample from an individual to be tested for the presence or absence of one or more nucleic acid molecules comprising a nucleic acid sequence selected from the group consisting of SEQ ID NOS: 1-20, 61-108, 205-273 and 412, wherein the absence of one or more of said nucleic acid sequences is indicative of an alteration in the human Y chromosome in the individual. While the presence or absence of a single nucleic acid molecule from this group can be informative, the greatest informational value is obtained when the presence or absence of multiple nucleic acid molecules is assessed in combination. That is, the greatest specificity of information can be obtained by assessing the pattern of presence and absence of particular markers. Preferred combinations will be apparent with reference to FIGS. 2, 3A-3B, 4A-4B and 8.

For example, with reference to FIG. 2, assessment of the presence or absence of a single marker, e.g., sY1317, is informative in that the absence of this marker is expected only in AZFa, Xp-→Xq(XG), Xp-→(KALP, VCY), and Iso Yp (centromere) alterations. Thus, absence of this marker indicates that one of these alterations is present in the tested Y chromosome.

However, the pattern of presence or absence of a set of markers provides more specific information regarding the particular alteration present in the sample. For example, again with reference to FIG. 2, an AZFa deletion is indicated by the absence of markers sY1317 and sY1234 along with the presence of the other indicated markers.

Thus, based on the additional markers assessed the type of alteration can be narrowed from a potential list of alterations (AZFa, Xp->Xq(XG), Xp->(KALP, VCY), and Iso Yp (centromere) alterations) to specific identification of an AZFa deletion. The same analytical framework can be applied to the other STSs shown in FIGS. 2, 3A-3B, 4A-4B and 8.

Accordingly, preferred methods of the invention include the assessment of multiple STSs in combination. For example, one preferred combination of markers to be assessed is all or a subset of nucleic acid molecules comprising SEQ ID NOS: 1-20, SEQ ID NOS: 61-108, SEQ ID NOS: 205-273 or SEQ ID NO: 412. Particularly useful subsets of these markers will be apparent from review of FIGS. 2, 3A-3B, 4A-4B and 8 and can be selected, for example, on the basis of the alteration to be assessed. For example, the control markers shown in the Figs. may be substituted or omitted in the judgment of the practitioner. In preferred embodiments the presence or absence of all of SEQ ID NOS: 1-20 or SEQ ID NOS: 1-20 and 412 is assessed, in other preferred embodiments the presence or absence of all of SEQ ID NOS: 61-108 is assessed, and in other preferred embodiments the presence or absence of all of SEQ ID NOS: 205-273 is assessed. Markers in addition to those described herein can also be assessed in conjunction with assessment of the markers described herein.

The Y chromosomal alteration to be detected (determined) can include any disruption of the chromosome, such as deletion of one or more nucleotides, addition of one or more nucleotides, or change in one or more nucleotides, including total loss of the chromosome. In preferred embodiments the alteration detected is a deletion, and more preferably one of the deletions shown in FIGS. 2, 3A-3B, 4A-4B and 8. The deletions shown in the Figs are referred to by their art-recognized names. For example, in FIG. 2 the AZFa deletion has been described in Sun et al., Hum. Mol. Genet., 9:2291-2296 (2000); the P5/proxP1 and P5/distP 1 deletions have been described in Repping et al., J. Hum. Genet., 71:906-922 (2002); the gr/gr and b1/b3 deletions have been described in Repping et al., Nature Genetics, 35:247-251 (2003); and the AFZc deletion has been described in Kuroda-Kawaguchi et al., Nature Genetics, 29:279-286 (2001).

The nucleic acid sample from the individual to be tested will preferably be genomic DNA and can be obtained from any nucleic acid source. The sample will preferably comprise human nucleic acid molecules in a form suitable for hybridization to probes and primers of the invention or will be treated to render the nucleic acid molecules suitable for hybridiation prior to carrying out the methods of the invention. The nucleic acid molecules in the sample may be isolated, cloned or amplified. As used herein, an “isolated” nucleic acid molecule is intended to mean a nucleic acid molecule which is not flanked by DNA sequences which normally (in nature) flank the nucleic acid molecule. Thus, an isolated nucleic acid molecule can include a nucleic acid molecule which is biologically isolated or synthesized chemically or by recombinant means.

Methods of isolating cell and tissue samples (sources of nucleic acid molecules) are well known to those of skill in the art and include, but are not limited to, scrapings, aspirations, tissue sections, needle biopsies, and the like. Frequently the sample will be a “clinical sample” which is a sample derived from a patient, including sections of tissues such as frozen sections or paraffin sections taken for histological purposes. The sample can also be derived from supernatants (of cells) or the cells themselves from cell cultures, cells from tissue culture and other media in which it may be desirable to detect chromosomal abnormalities. In some cases, the nucleic acids may be amplified using standard techniques such as PCR, prior to carrying out the methods of the invention. The sample may be isolated nucleic acid molecules immobilized on a solid. The sample may also be prepared such that individual nucleic acids remain substantially intact. Suitable sources include, but are not limited to, blood, skin, sperm, hair root, saliva or buccal cells, or cells cultured from blood or skin.

The nucleic acid sample will be obtained from a human male, typically from a human male who is part of a couple having difficulty conceiving a child. Even more typically the male will have been, at least preliminarily, determined to have a reduced sperm count. Reduced sperm count is understood to encompass both oligospermia and azoospermia, i.e., a sperm count of less than 20 million per ml, including total absence of sperm. Azoospermia is defined as a condition wherein the concentration of sperm in a semen sample is 0 to occasional sperm per ml, and oligospermia is defined as a condition wherein the concentration of sperm in a semen sample ranges from occasional to less than 20 million per ml.

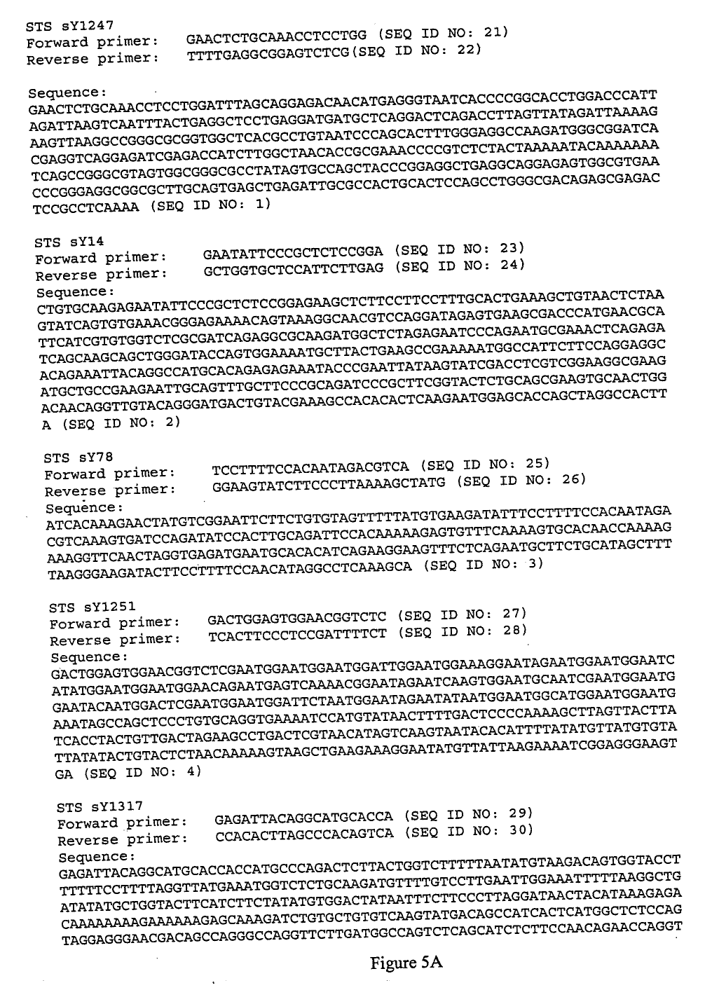

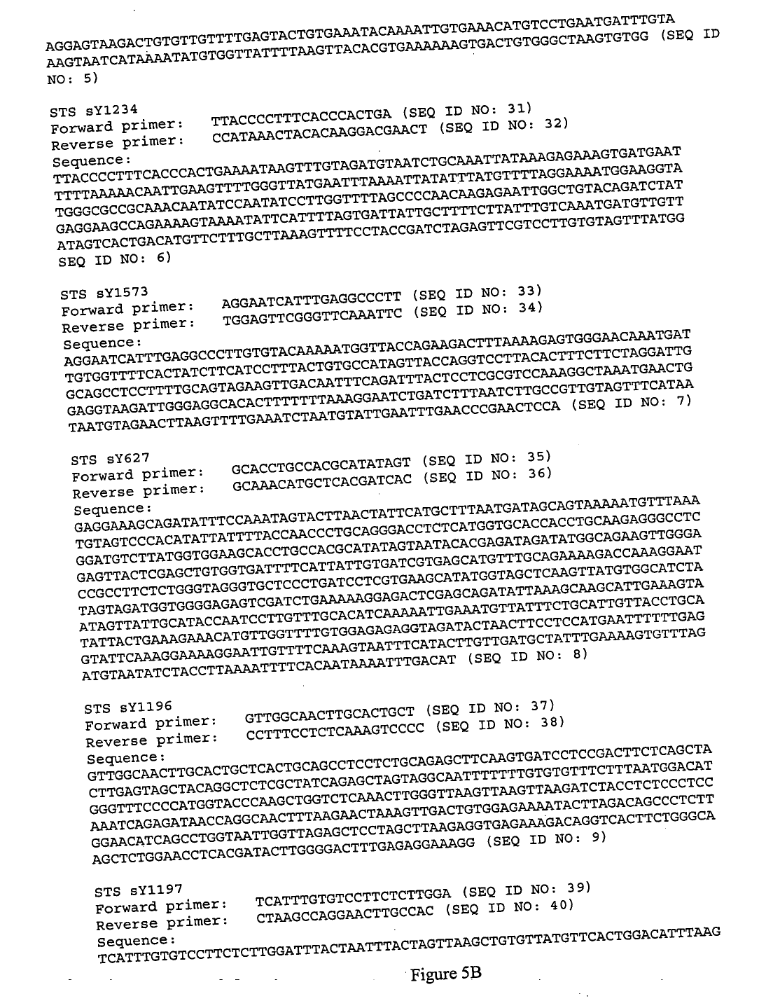

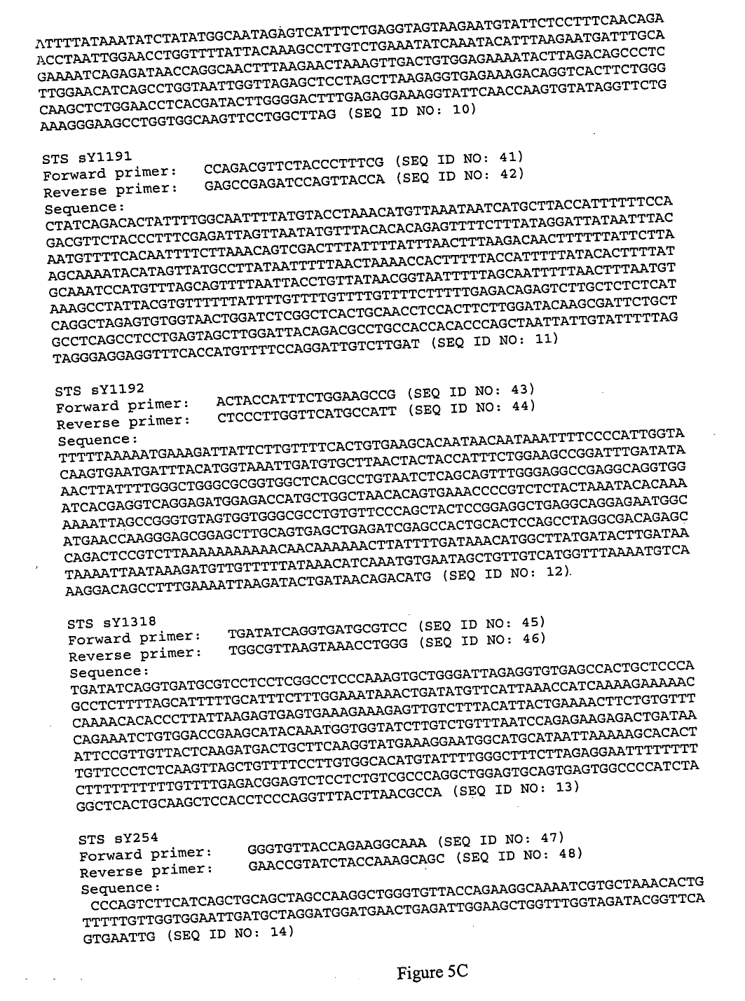

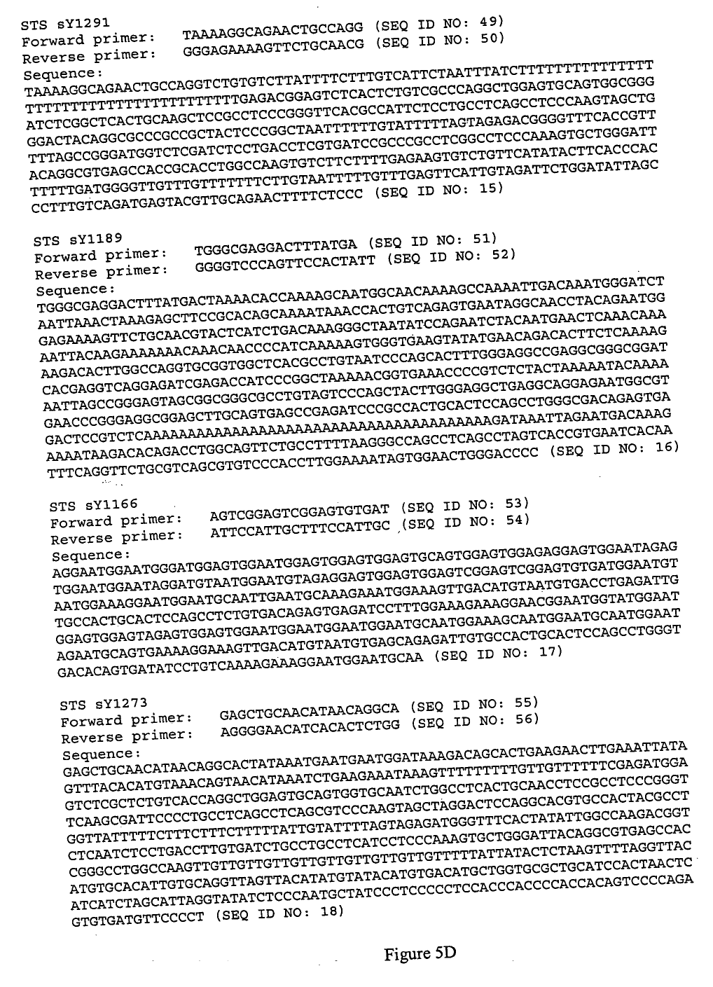

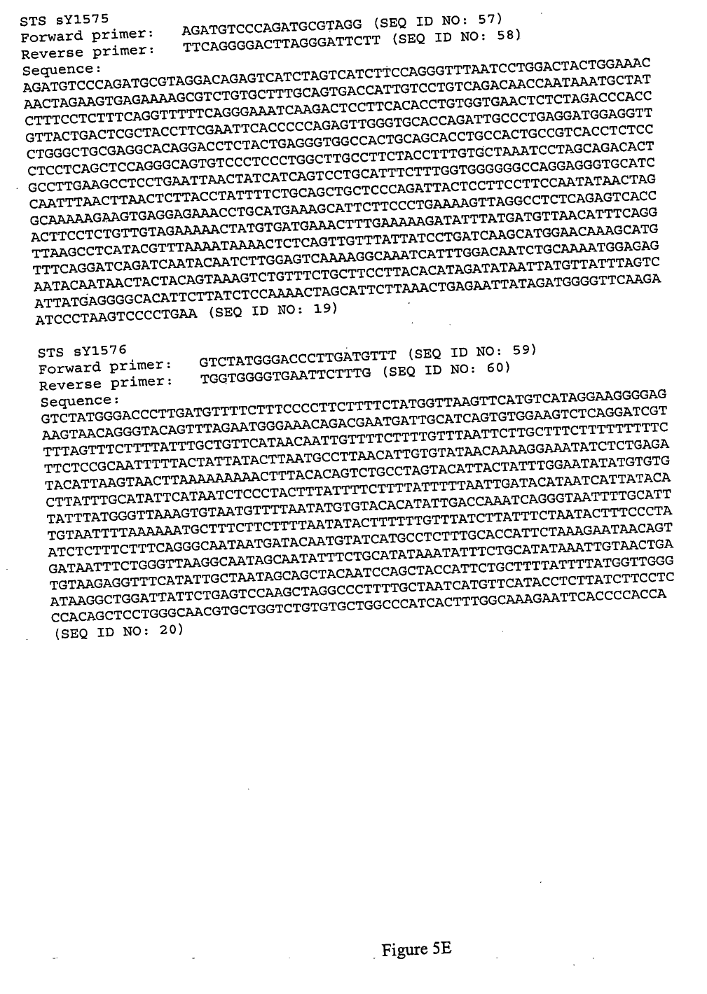

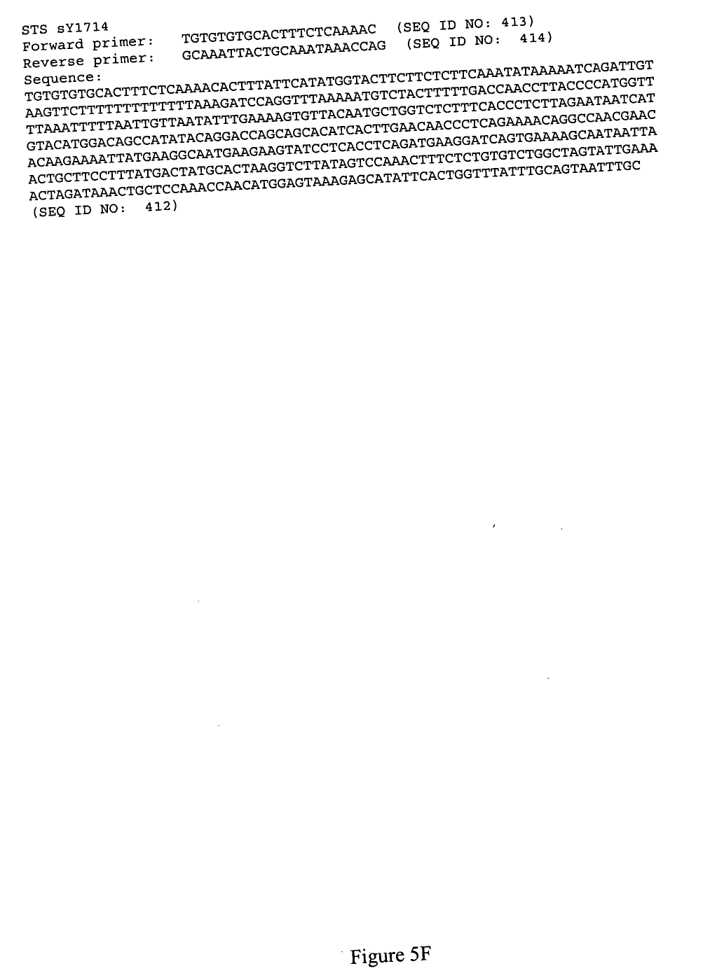

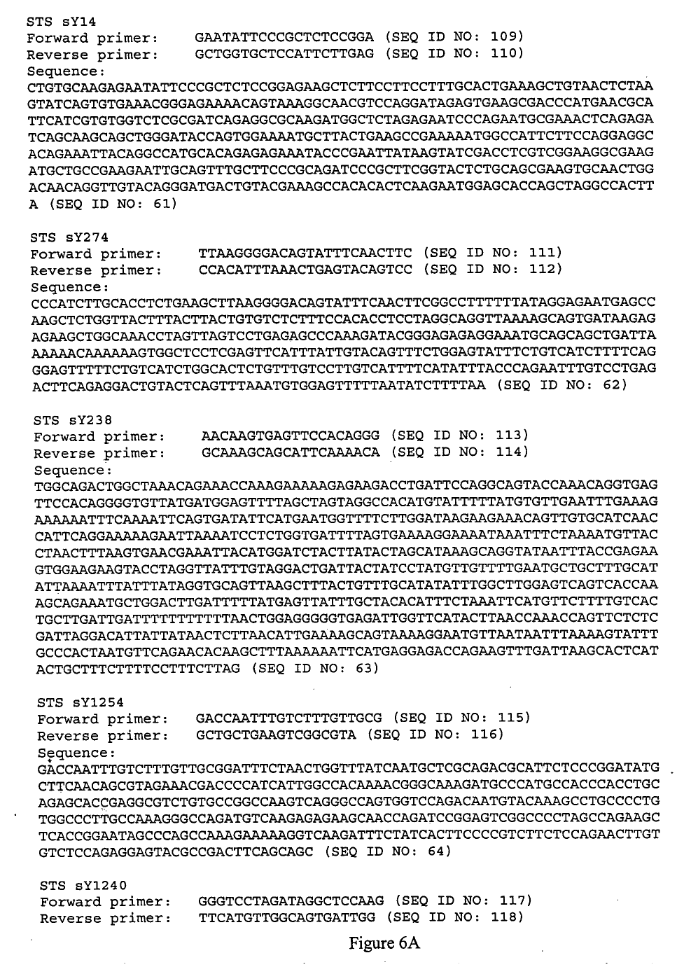

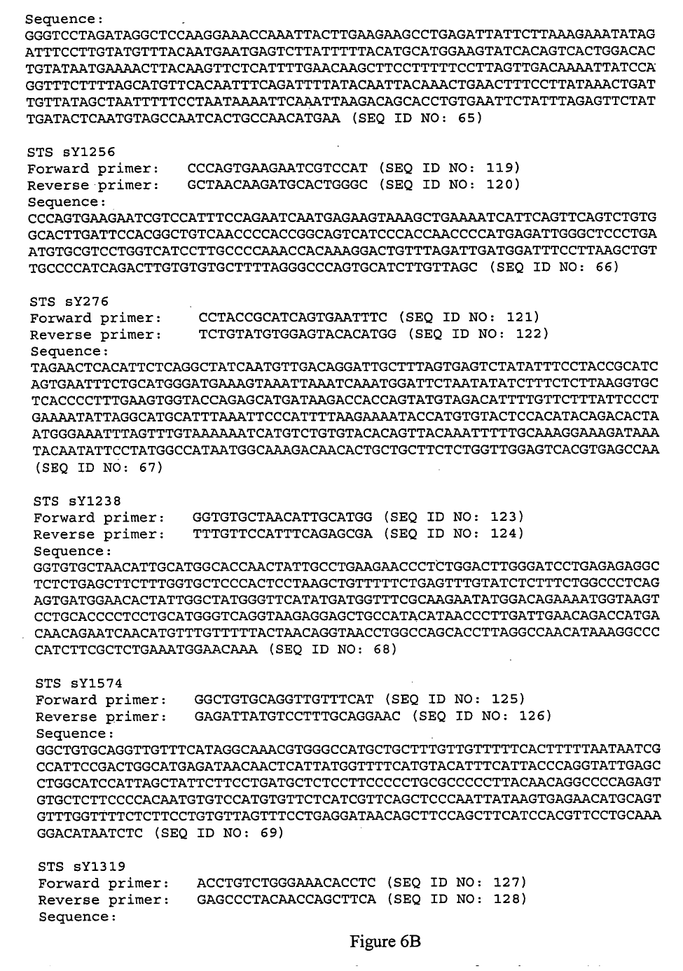

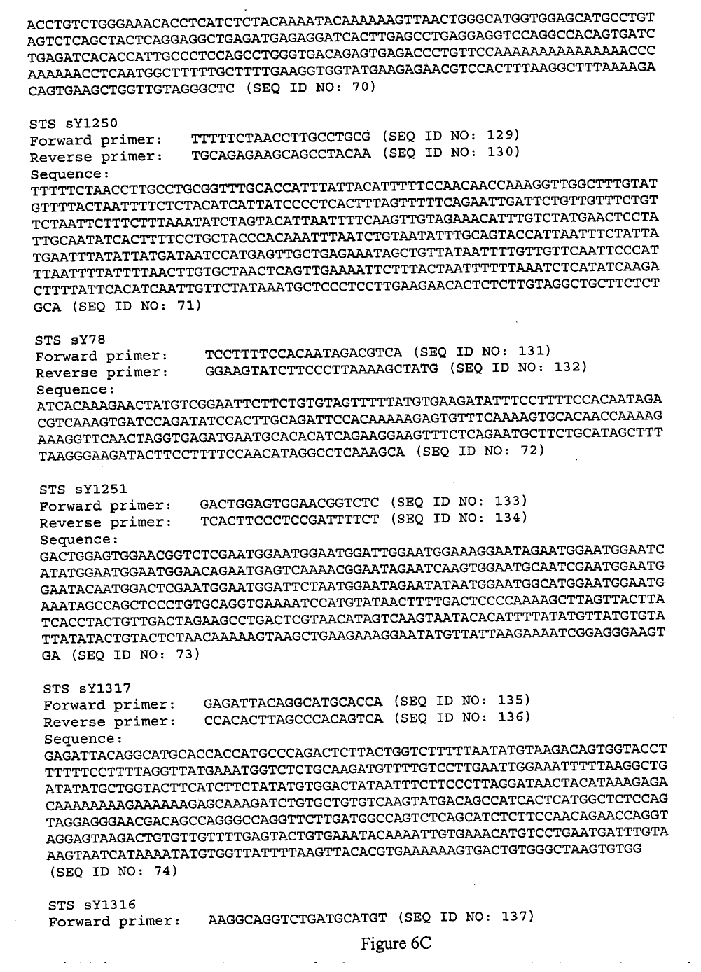

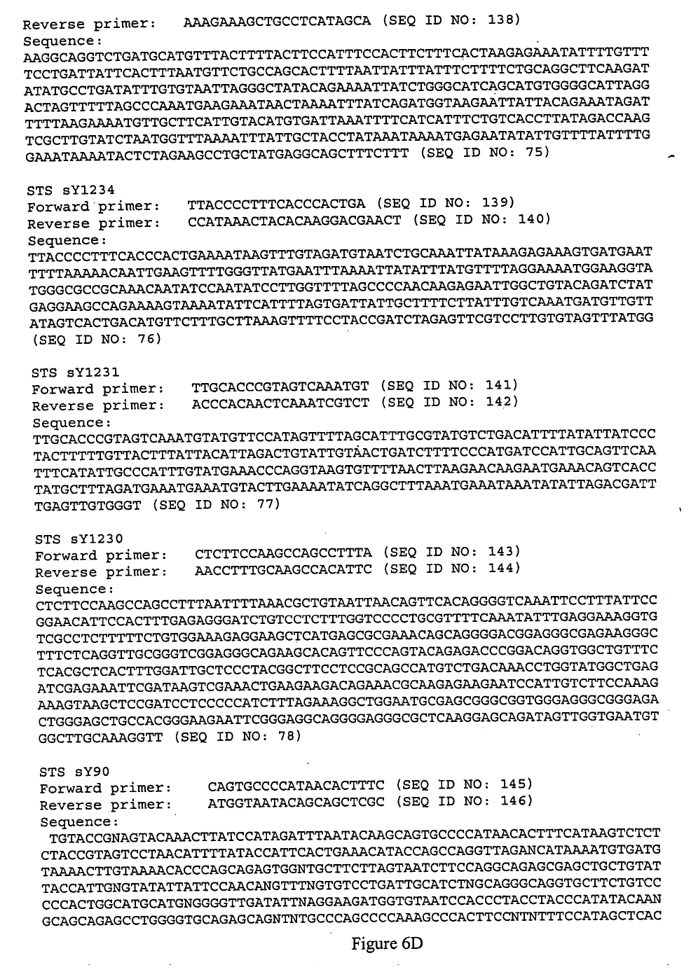

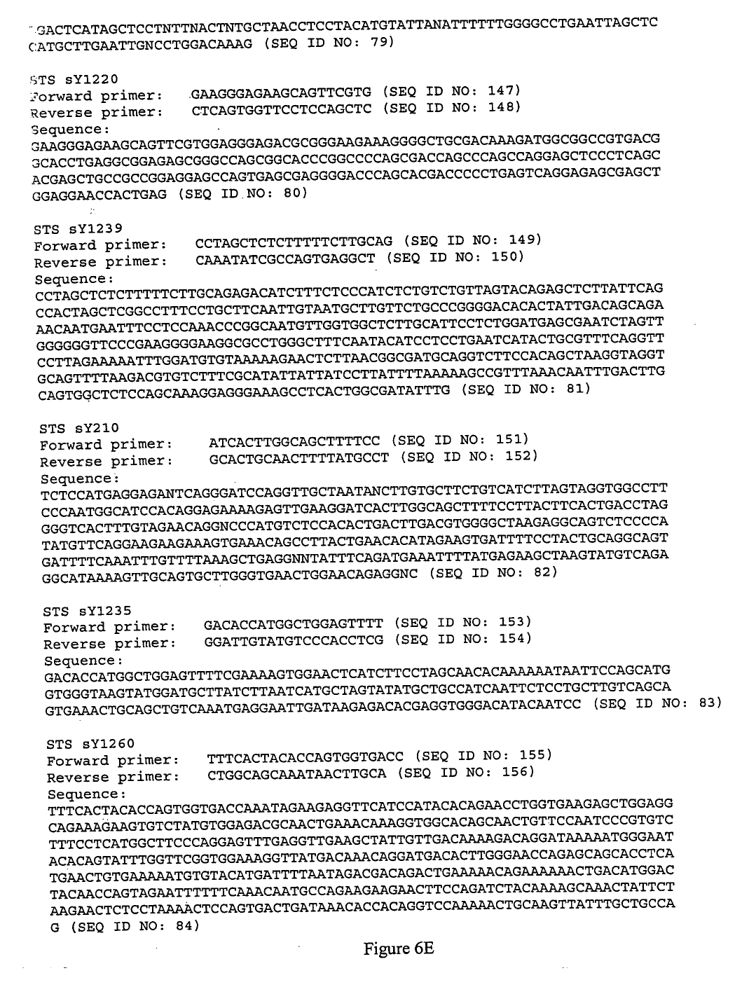

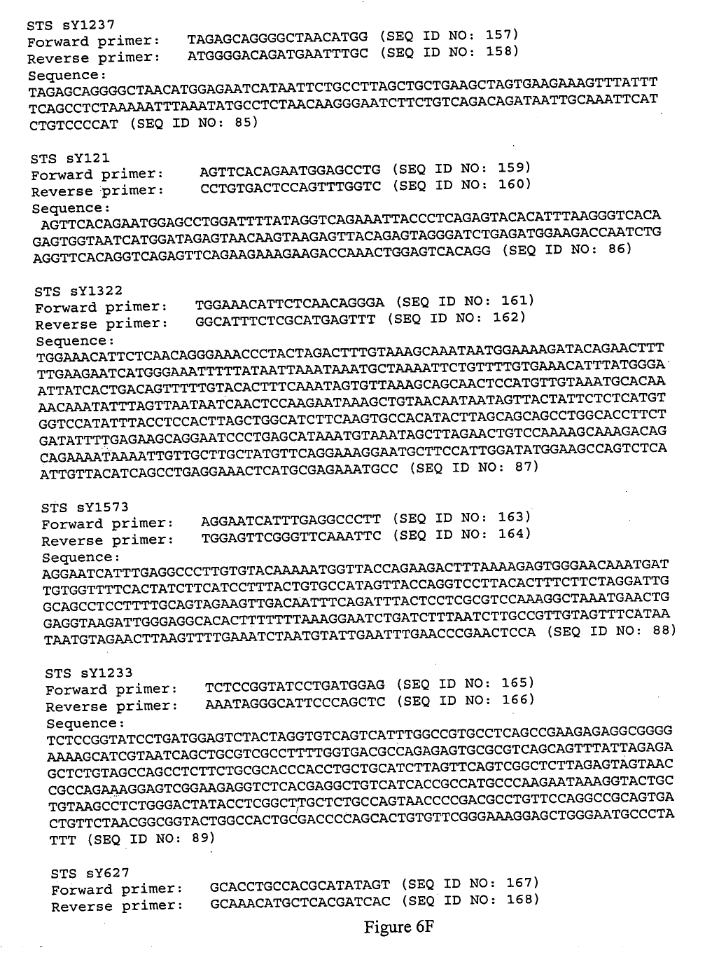

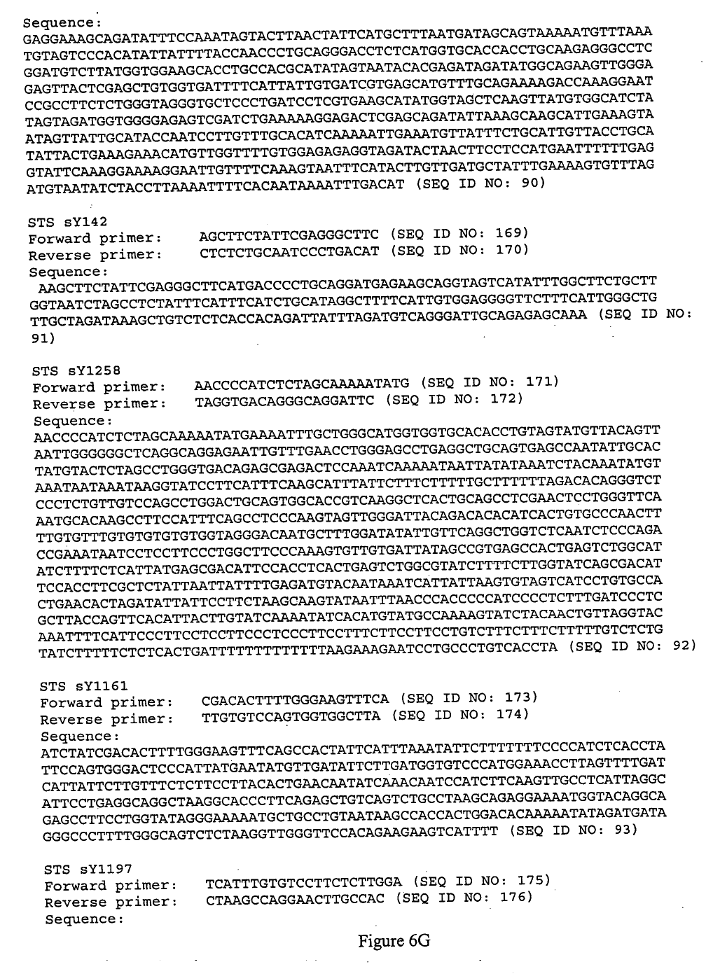

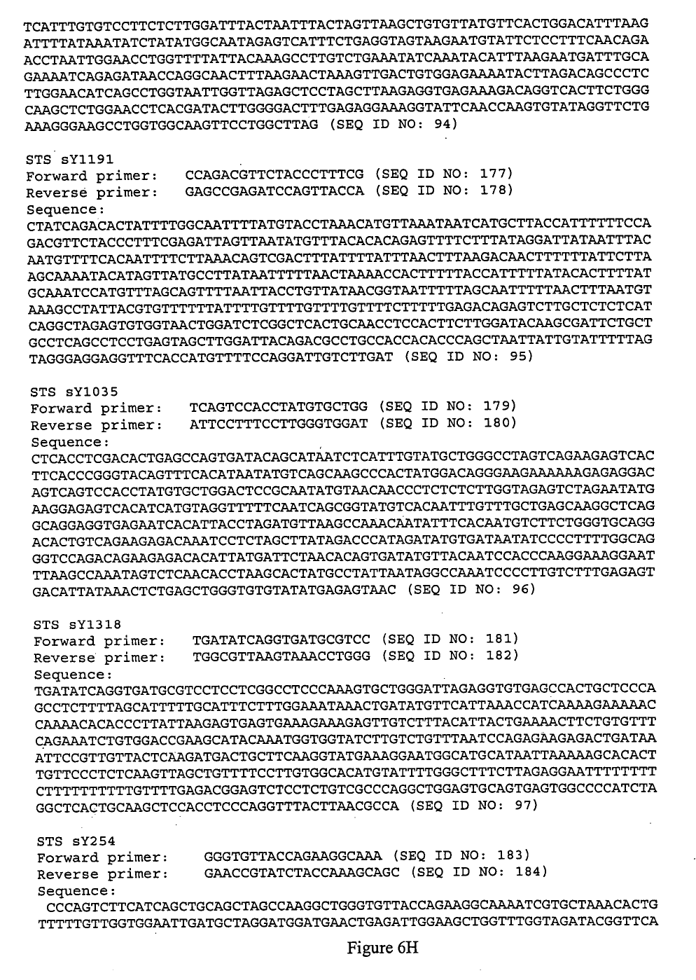

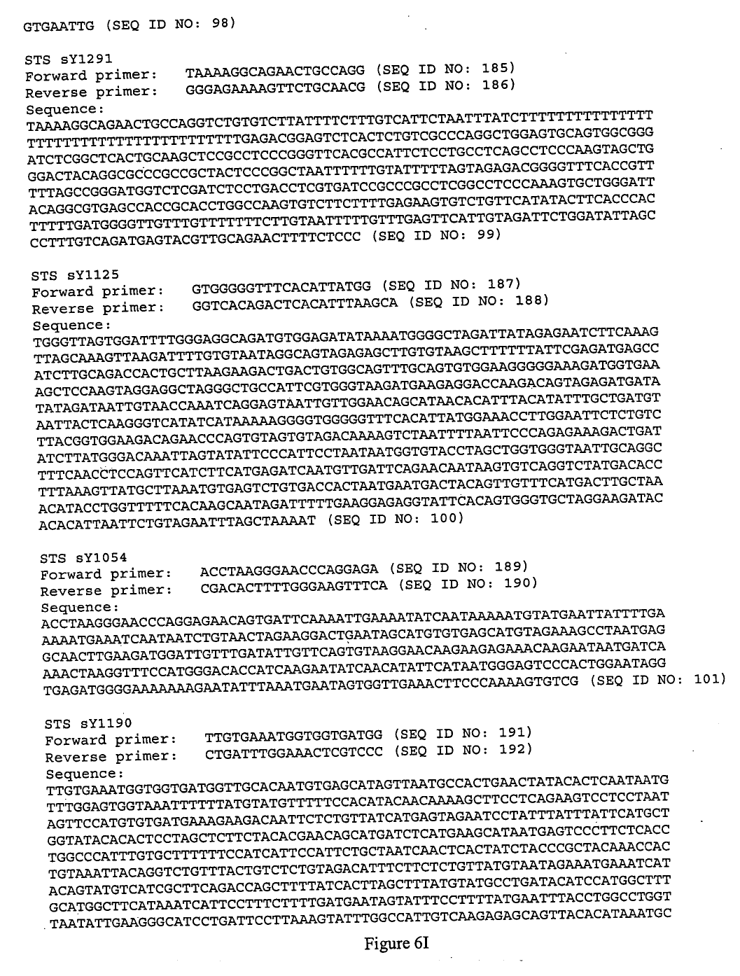

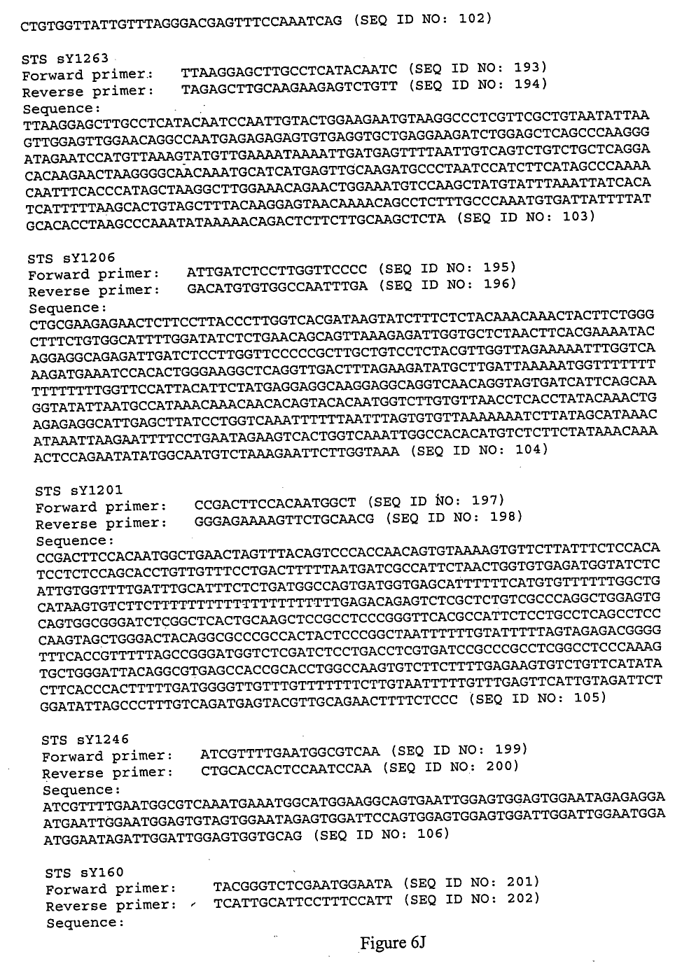

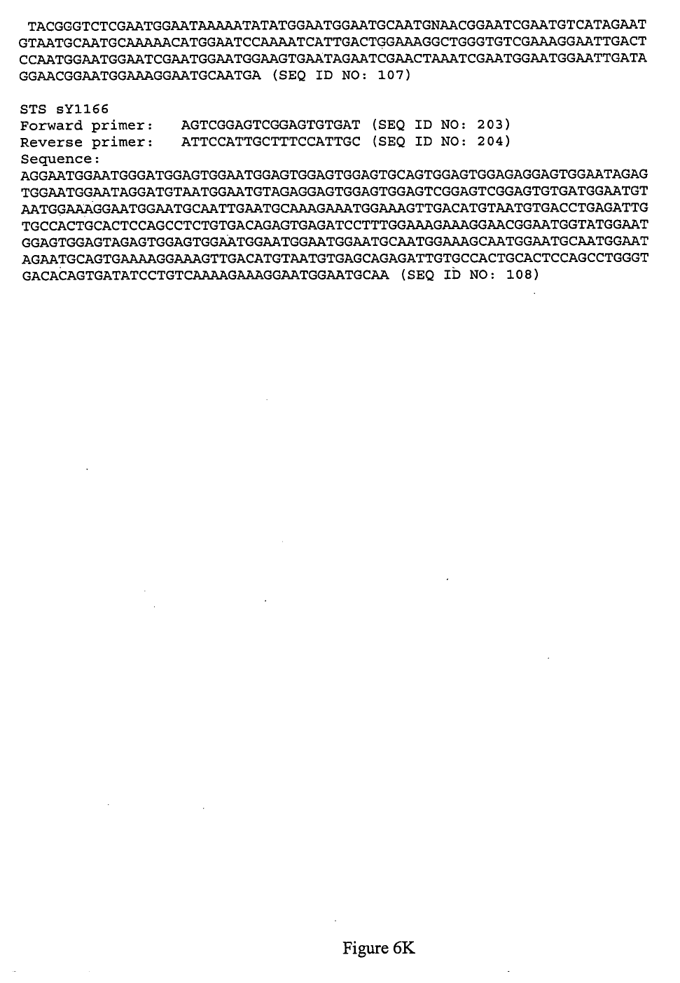

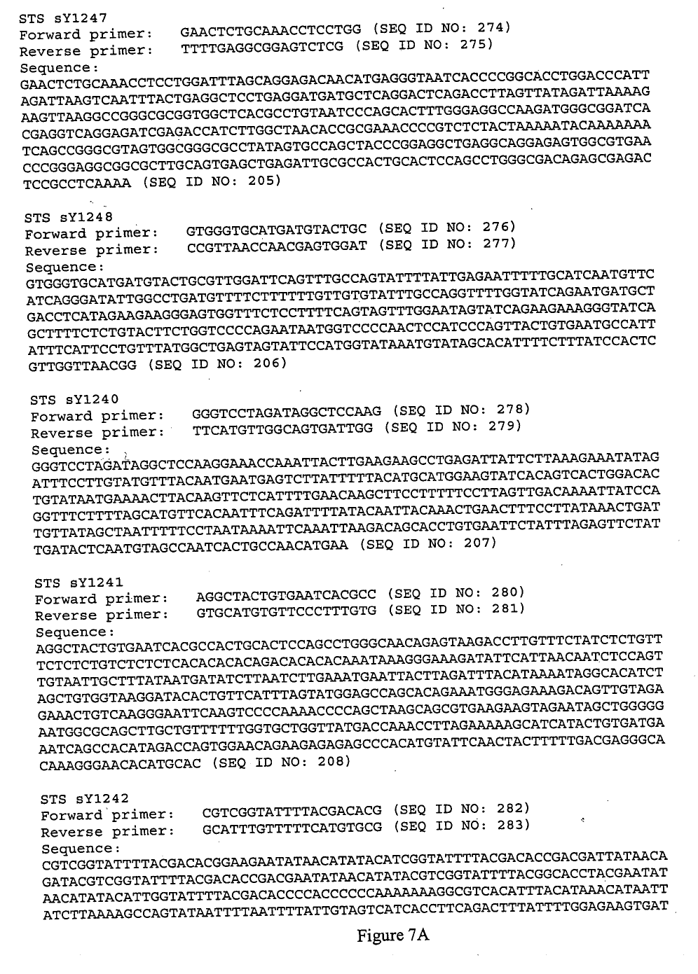

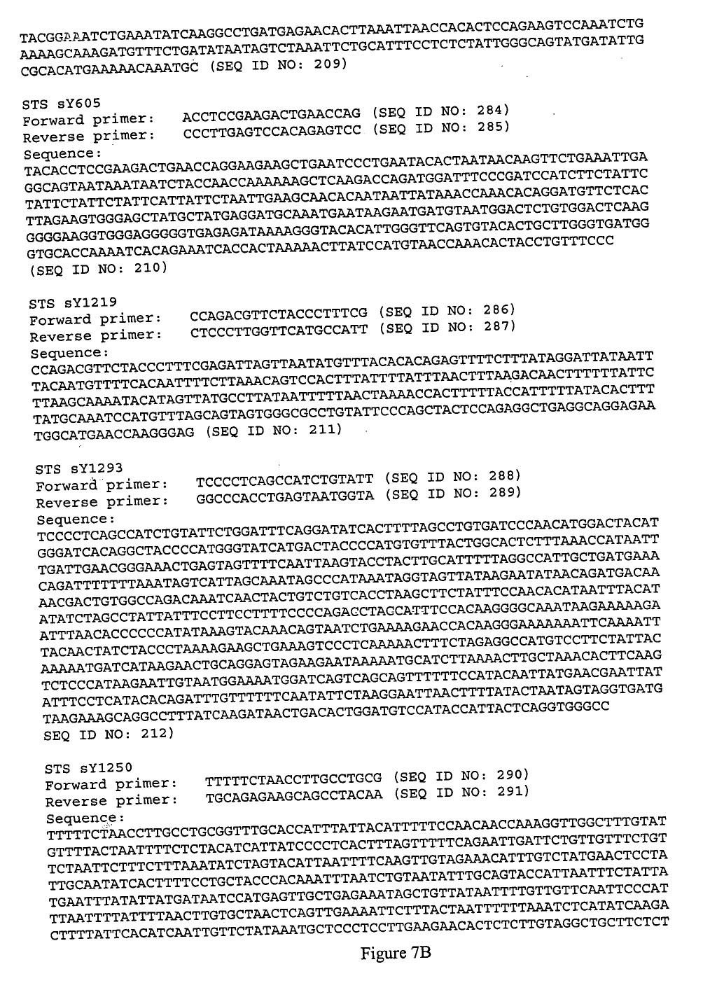

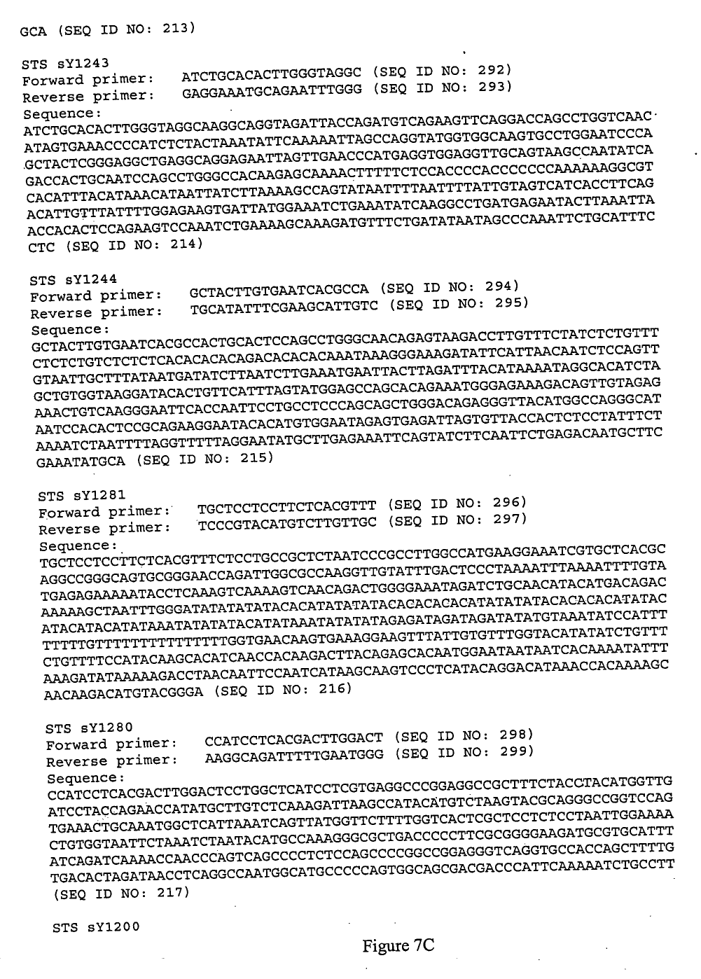

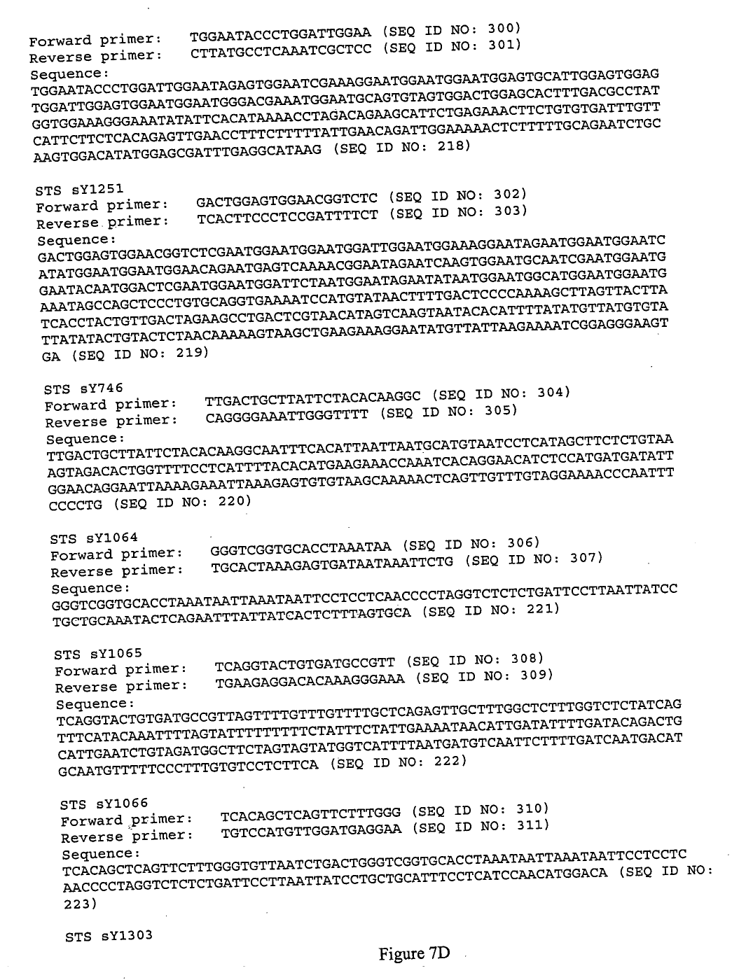

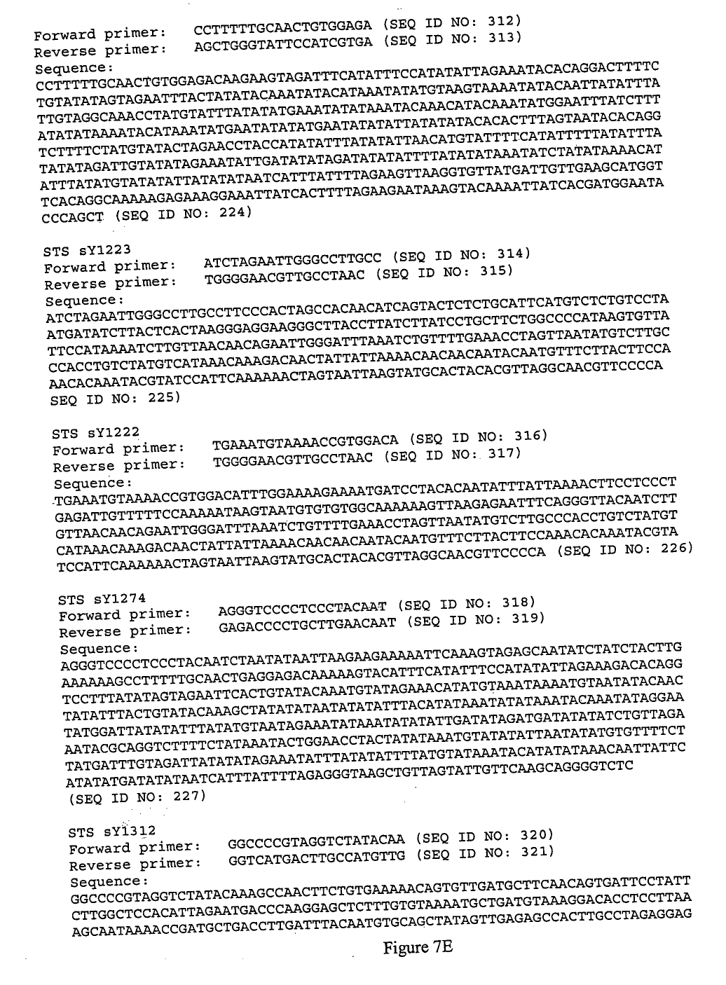

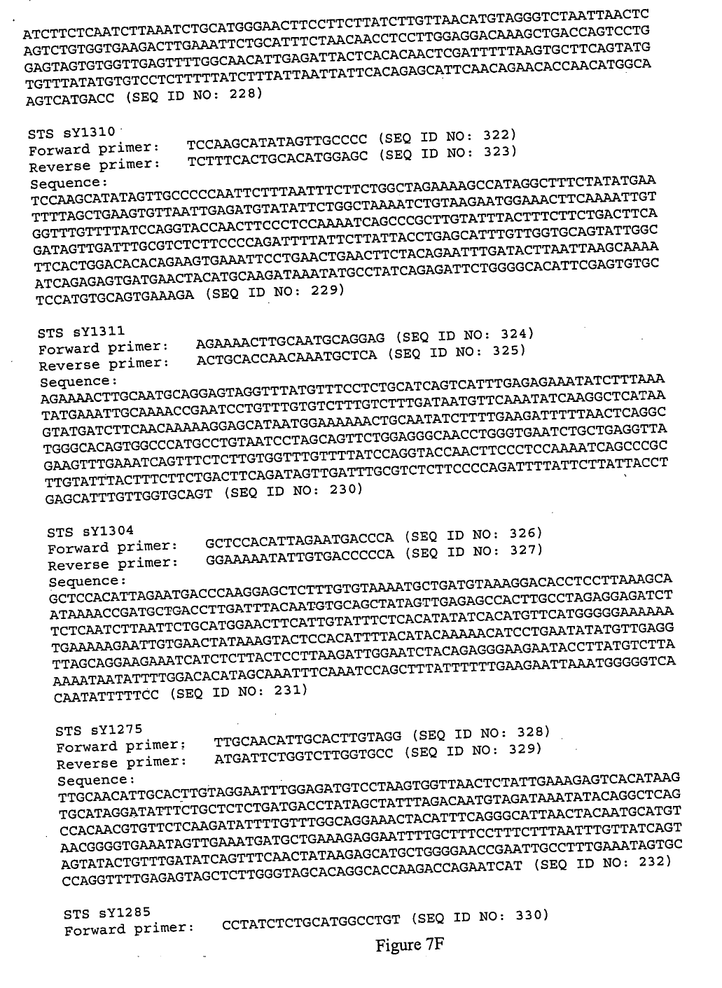

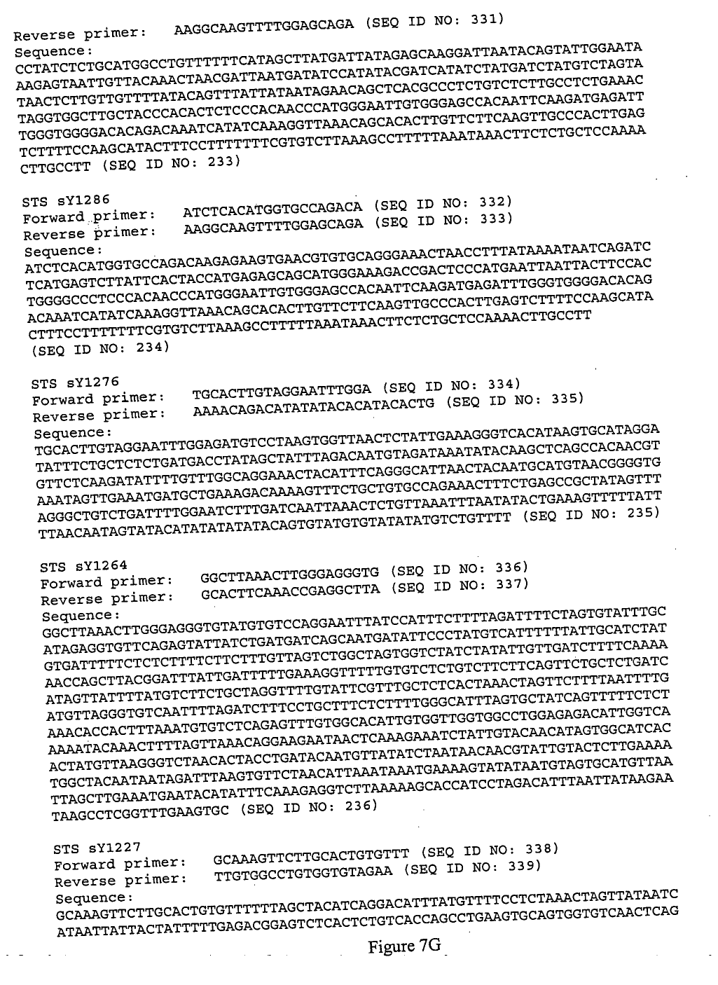

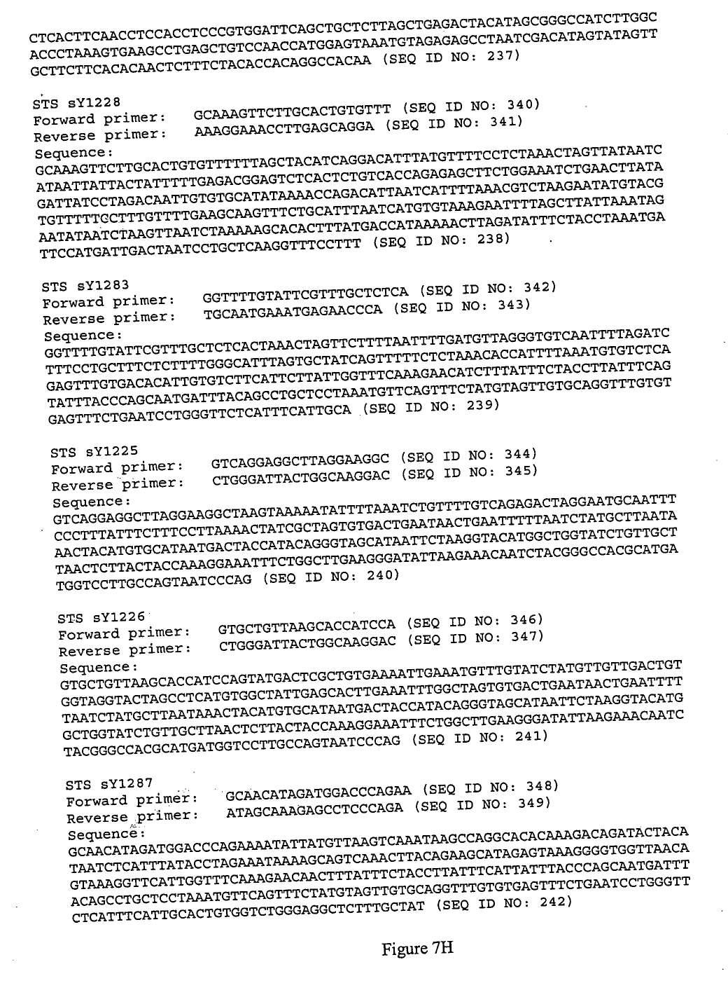

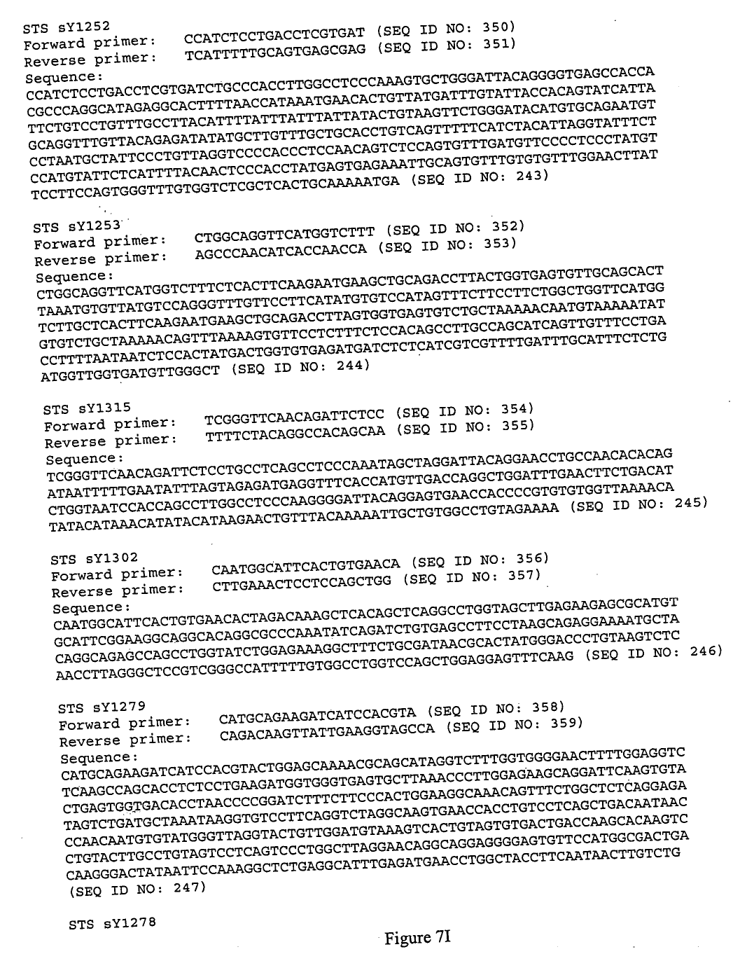

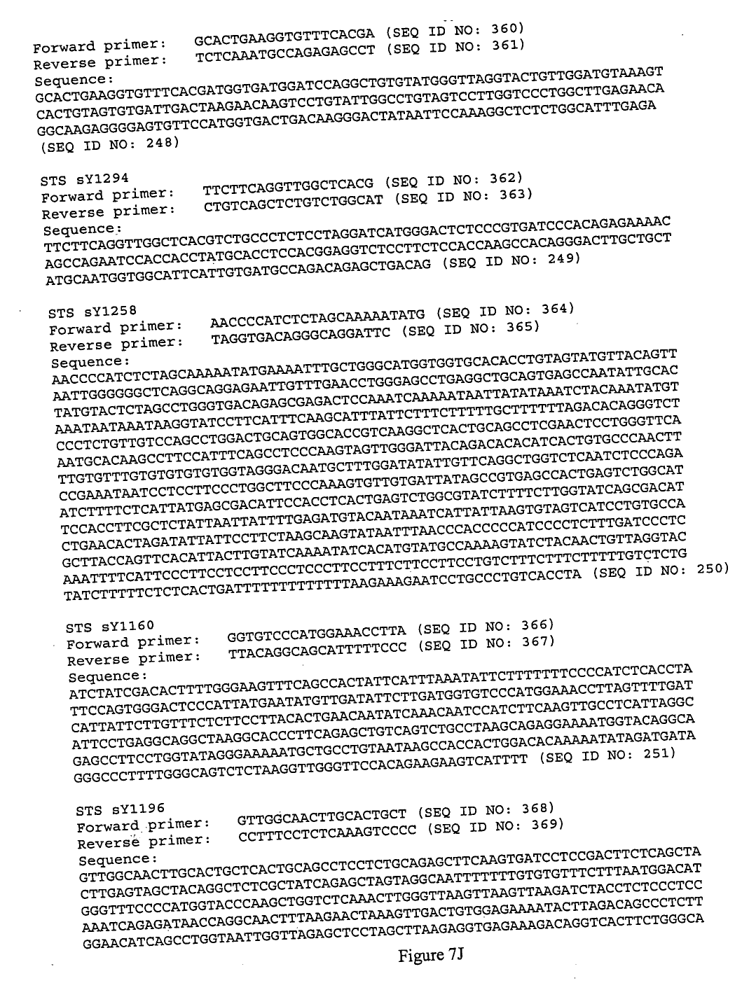

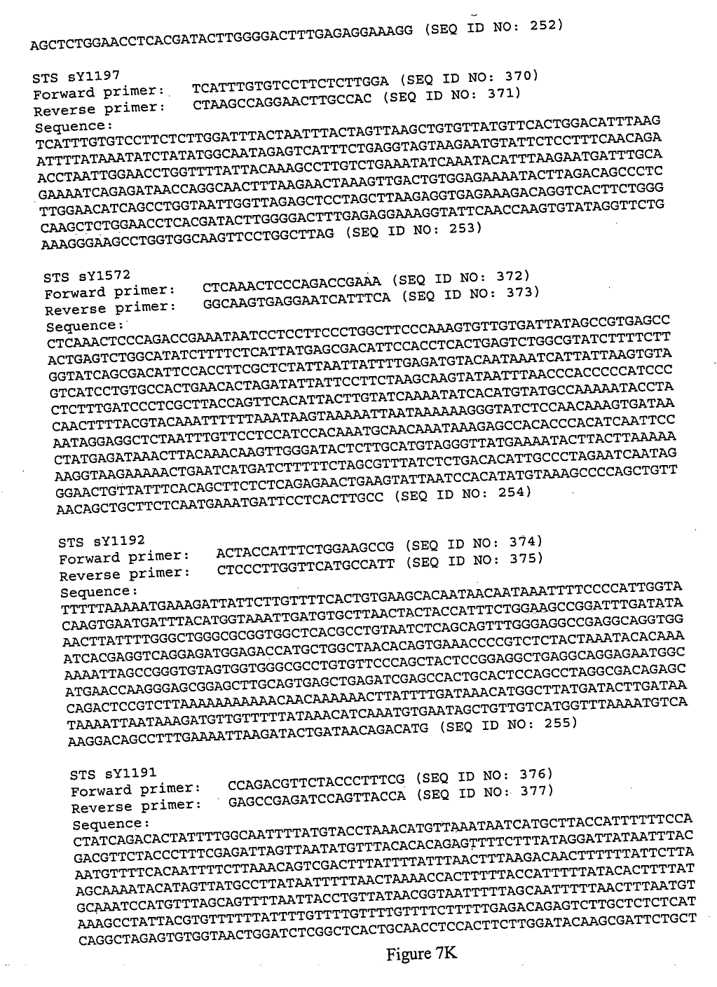

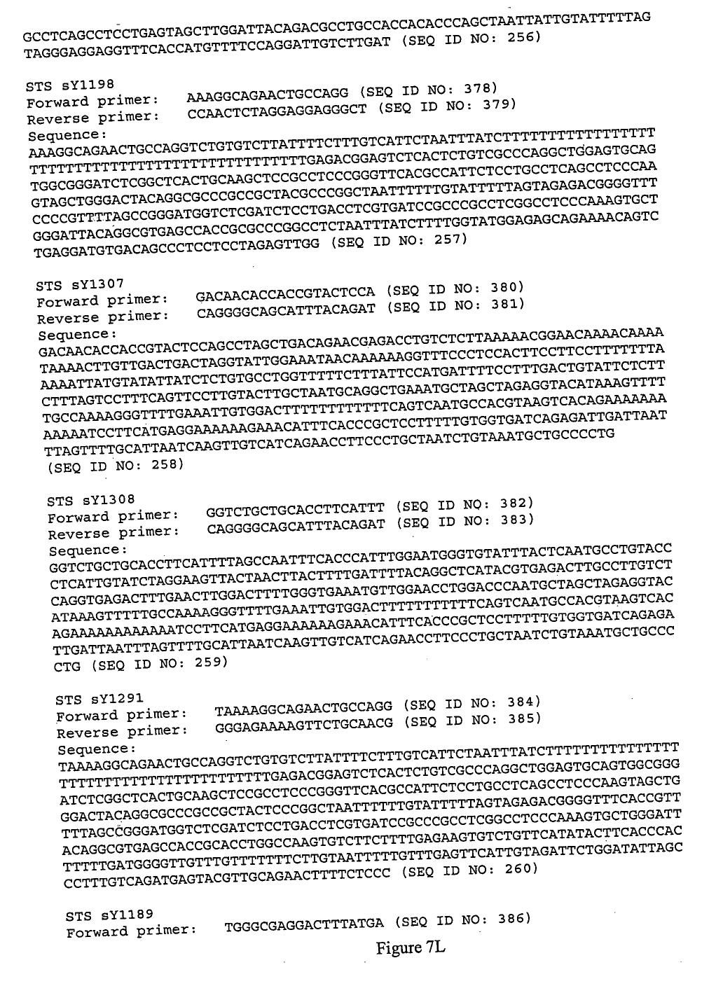

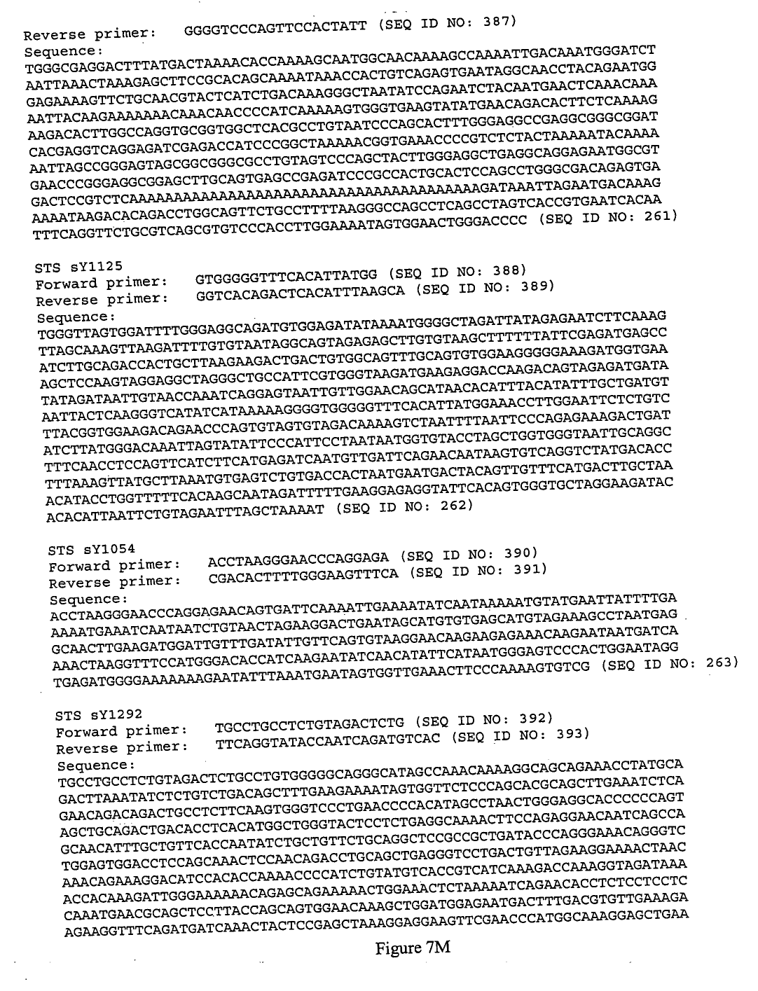

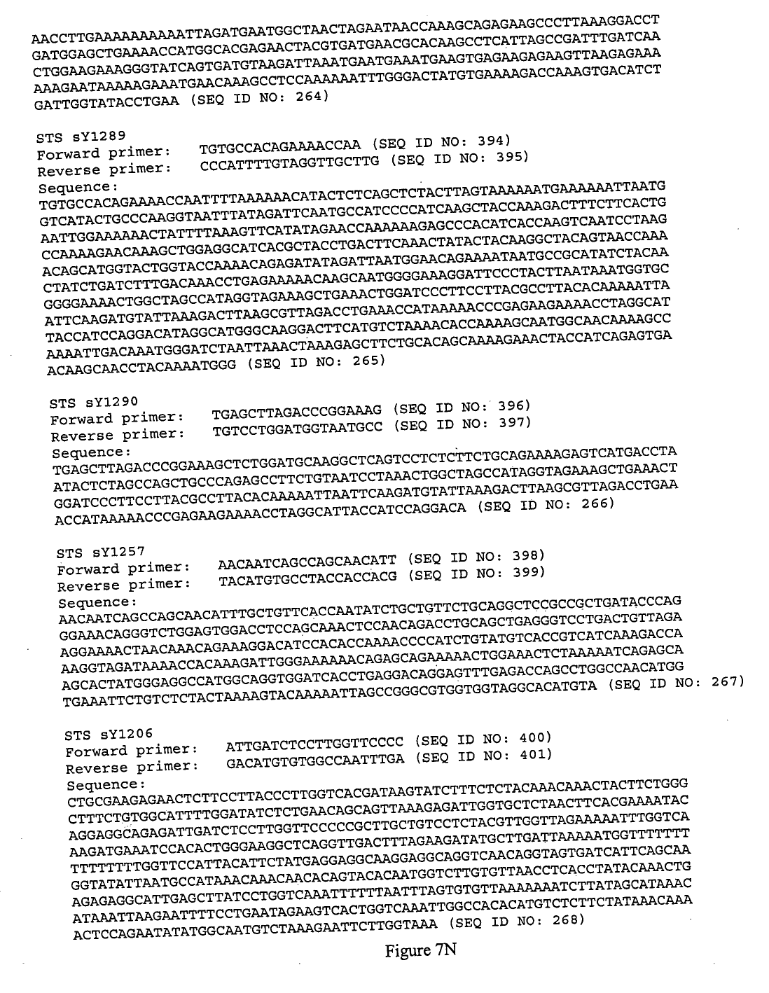

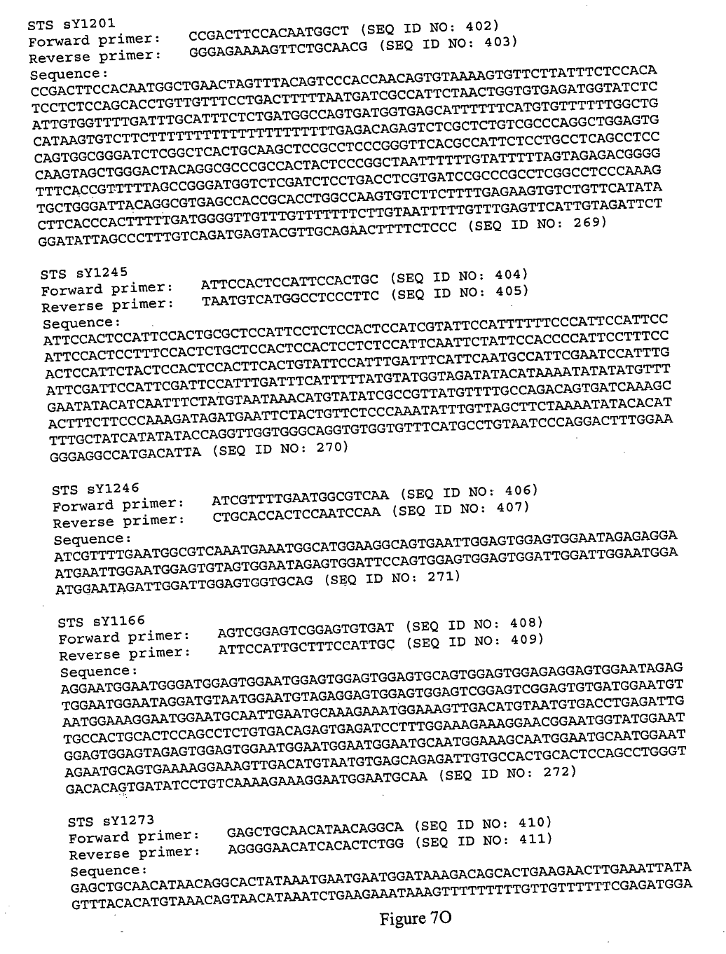

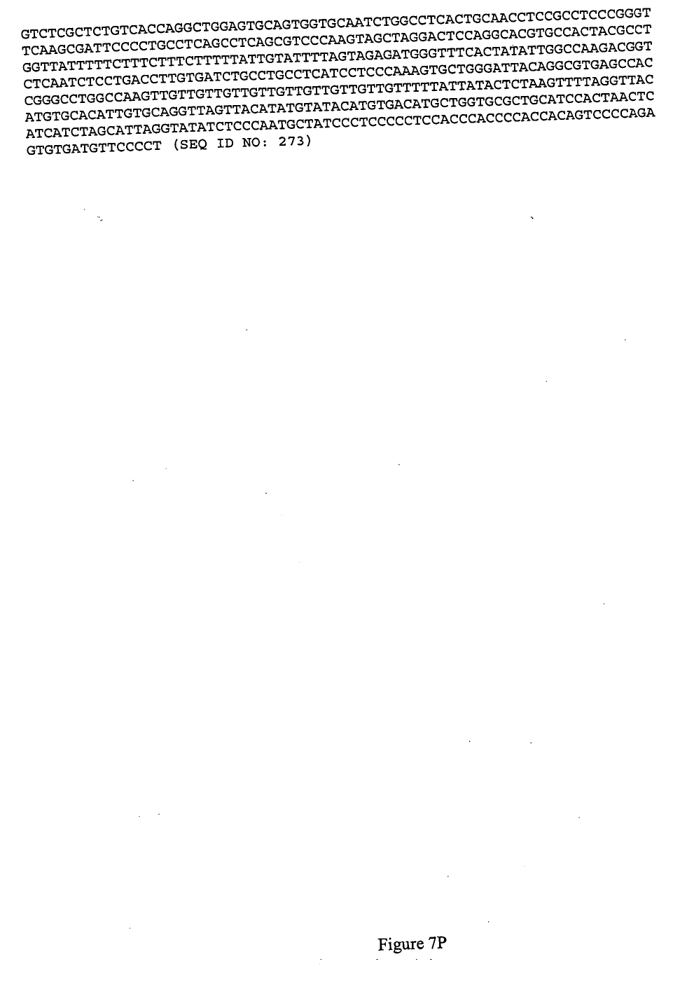

The nucleic acid sequences of markers of the invention are shown in FIGS. 5A-5F, 6A-6K and 7A-7P. In particular methods of the invention the presence or absence of the markers of the invention can be assessed (determined, analyzed) by any methods known in the art. In particular embodiments the presence or absence is determined by hybridization to specific probes and/or by amplification using specific primers. Based on the nucleic acid sequences of the markers shown in FIGS. 5A-5F, 6A-6K and 7A-7P, suitable probes and primers can readily be designed by the skilled artisan.

In one embodiment, the presence or absence of one or more markers is determined by amplification using specific primers. One or more markers of the invention can be amplified using primer pairs that include or flank the marker sequence. Particularly preferred primers identified as specifically priming the markers of the invention are shown in FIGS. 5A-5F, 6A-6K and 7A-7P. Other primers can readily be designed by the skilled artisan. For example, the primers selected from the group consisting of SEQ ID NOS: 21-60 can be used to determine the presence or absence of a corresponding nucleic acid molecule comprising a nucleic acid sequence selected from the group consisting of SEQ ID NOS: 1-20, and the primers selected from the group consisting of SEQ ID NOS: 109-204 can be used to determine the presence or absence of a corresponding nucleic acid molecule comprising a nucleic acid sequence selected from the group consisting of SEQ ID NOS: 61-108. Similarly, primers selected from the group consisting of SEQ ID NOS: 274-411 can be used to determine the presence or absence of a corresponding nucleic acid molecule comprising a nucleic acid sequence selected from the group consisting of SEQ ID NOS: 205-273, and primers selected from the group consisting of SEQ ID NOS: 413-414 can be used to determine the presence or absence of a corresponding nucleic acid molecule comprising SEQ ID NO: 412.

FIGS. 5A-5F, 6A-6K and 7A-7P show the relationship between the primers of SEQ ID NOS 21-60 and 413-414 and the STSs of SEQ ID NOS: 1-20 and 412, respectively, the primers of SEQ ID NOS: 109-204 and the STSs of SEQ ID NOS: 61-108, and the primers of SEQ ID NOS: 274-411 and the STSs of SEQ ID NOS: 205-273, respectively. As used herein, a primer “corresponds” to an STS if it serves as a specific primer for that STS in an amplification reaction. For example, SEQ ID NOS: 21 and 22 are primers which serve as specific primers for SEQ ID NO: 1, and thus SEQ ID NOS: 21 and 22 are primers which correspond to the STS of SEQ ID NO: 1.

Suitable amplification methods include, but are not limited to: polymerase chain reaction, PCR (PCR Protocols, A Guide to Methods and Applications, ed. Innis, Academic Press, N.Y. (1990) and PCR Strategies (1995), ed. Innis, Academic Press, Inc., N.Y. (Innis)); ligase chain reaction (LCR) (Wu (1989) Genomics 4:560; Landegren (1988) Science 241:1077; Barringer (1990) Gene 89:117); transcription amplification (Kwoh (1989) Proc. Natl. Acad. Sci. USA 86:1173); and self-sustained sequence replication (Guatelli (1990) Proc. Natl. Acad. Sci. USA, 87:1874); Q Beta replicase amplification and other RNA polymerase mediated techniques (e.g., NASBA, Cangene, Mississauga, Ontario); see Berger (1987) Methods Enzymol. 152:307-316, Sambrook, and Ausubel, as well as Mullis (1987) U.S. Pat. Nos. 4,683,195 and 4,683,202; Arnheim (1990) C&EN 36-47; Lomell (1989) J. Clin. Chem. 35:1826; Van Brunt (1990) Biotechnology 8:291-294; Wu (1989) Gene 4:560; Sooknanan (1995) Biotechnology 13:563-564. Methods for cloning in vitro amplified nucleic acids are described in Wallace, U.S. Pat. No. 5,426,039. Methods of amplifying large nucleic acids are summarized in, e.g., Cheng (1994) Nature 369:684-685.

The presence or absence of amplification products for one or more markers can be analyzed to identify the presence or absence of one or more markers of the invention. That is, if an amplification product for a particular marker is detected, it can be concluded that that marker is present in the sample, and if an amplification product is not detected it can be concluded that the marker is not present in the sample.

In another method of the invention, the presence or absence of one or more markers of the invention is determined using nucleic acid probes which specifically hybridize to the markers of the invention. The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. A “stringent hybridization” and “stringent hybridization wash conditions” in the context of nucleic acid hybridization (e.g., as in array, Southern or Northern hybridizations) are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, “Overview of principles of hybridization and the strategy of nucleic acid probe assays,” Elsevier, N.Y. (“Tijssen”). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook (1989) Molecular Cloning: A Laboratory Manual (2nd ed.) Vol. 1-3, Cold Spring Harbor Laboratory, Cold Spring Harbor Press, NY (“Sambrook”). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes.

Nucleic acid hybridization assays can be performed in an array-based format. Arrays are a multiplicity of different “probe” or “target” nucleic acids (or other compounds) which hybridize with a sample nucleic acid. In an array format a large number of different hybridization reactions can be run essentially “in parallel.” This provides rapid, essentially simultaneous, evaluation of a large number of loci. Methods of performing hybridization reactions in array based formats are also described in, e.g., Pastinen (1997) Genome Res. 7:606-614; Jackson (1996) Nature Biotechnology 14:1685; Chee (1995) Science 274:610; and WO 96/17958.

Many methods for immobilizing nucleic acids on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials which may be employed include paper, ceramics, metals, metalloids, semiconductive materials, cermets or the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, enhance signal detection or the like. If covalent bonding between a compound and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature. For example, methods for immobilizing nucleic acids by introduction of various functional groups to the molecules is known (see, e.g., Bischoff (1987) Anal. Biochem., 164:336-344; Kremsky (1987) Nucl. Acids Res. 15:2891-2910). Modified nucleotides can be placed on the target using PCR primers containing the modified nucleotide, or by enzymatic end labeling with modified nucleotides. Use of membrane supports (e.g., nitrocellulose, nylon, polypropylene) for the nucleic acid arrays of the invention is advantageous because of well developed technology employing manual and robotic methods of arraying targets at relatively high element densities. Such membranes are generally available and protocols and equipment for hybridization to membranes is well known.

Target elements of various sizes, ranging from 1 mm diameter down to 1 um can be used with these materials. Smaller target elements containing low amounts of concentrated, fixed probe DNA are used for high complexity comparative hybridizations since the total amount of sample available for binding to each target element will be limited. Thus it is advantageous to have small array target elements that contain a small amount of concentrated probe DNA so that the signal that is obtained is highly localized and bright. Such small array target elements are typically used in arrays with densities greater than 10.sup.4/cm.sup.2. Relatively simple approaches capable of quantitative fluorescent imaging of 1 cm.sup.2 areas have been described that permit acquisition of data from a large number of target elements in a single image (see, e.g., Wittrup (1994) Cytometry 16:206-213).

Arrays on solid surface substrates with much lower fluorescence than membranes, such as glass, quartz, or small beads, can achieve much better sensitivity. Substrates such as glass or fused silica are advantageous in that they provide a very low fluorescence substrate, and a highly efficient hybridization environment. Covalent attachment of the target nucleic acids to glass or synthetic fused silica can be accomplished according to a number of known techniques (described above). Nucleic acids can be conveniently coupled to glass using commercially available reagents. For instance, materials for preparation of silanized glass with a number of functional groups are commercially available or can be prepared using standard techniques (see, e.g., Gait (1984) Oligonucleotide Synthesis: A Practical Approach, IRL Press, Wash., D.C.). Quartz cover slips, which have at least 10-fold lower autofluorescence than glass, can also be silanized.

Alternatively, probes can also be immobilized on commercially available coated beads or other surfaces. For instance, biotin end-labeled nucleic acids can be bound to commercially available avidin-coated beads. Streptavidin or anti-digoxigenin antibody can also be attached to silanized glass slides by protein-mediated coupling using e.g., protein A following standard protocols (see, e.g., Smith (1992) Science 258:1122-1126). Biotin or digoxigenin end-labeled nucleic acids can be prepared according to standard techniques. Hybridization to nucleic acids attached to beads is accomplished by suspending them in the hybridization mix, and then depositing them on the glass substrate for analysis after washing. Alternatively, paramagnetic particles, such as ferric oxide particles, with or without avidin coating, can be used.

In this embodiment of the invention, specific hybridization of a probe to one or more markers of the invention in a nucleic acid sample is indicative of the presence of that marker in the sample, and absence of specific hybridization of a probe to one or more markers of the invention in a nucleic acid sample is indicative of the absence of that marker in the sample.

In some embodiments of the invention, probes and primers of the invention are detectably labelled. The term “detectably labelled” as used herein refers to a nucleic acid attached to a detectable composition, i.e., a label. The detection can be by, e.g., spectroscopic, photochemical, biochemical, immunochemical, physical or chemical means. For example, useful labels include ³²P, ³⁵S, ³H, ¹⁴C, ¹²⁵I, ¹³¹I; fluorescent dyes (e.g., FITC, rhodamine, lanthanide phosphors, Texas red), electron-dense reagents (e.g. gold), enzymes, e.g., as commonly used in an ELISA (e.g., horseradish peroxidase, beta-galactosidase, luciferase, alkaline phosphatase), calorimetric labels (e.g., colloidal gold), magnetic labels (e.g. Dynabeads™.), biotin, dioxigenin, or haptens and proteins for which antisera or monoclonal antibodies are available. The label can be directly incorporated into the nucleic acid molecule to be detected, or it can be attached to a probe or antibody which hybridizes or binds to the target.

In various embodiments, the labels may be coupled to the probes and primers in a variety of ways known to those of skill in the art. Methods of labeling nucleic acids are well known to those of skill in the art. In various embodiments, the nucleic acid probes are labeled using nick translation, PCR, or random primer extension (see, e.g., Sambrook). Preferred labels are those that are suitable for use in arrays and in situ hybridization. In one embodiment, the nucleic acid probes or primers of the invention are detectably labeled. Alternatively, a detectable label which binds to a hybridization product may be used. Such detectable labels include any material having a detectable physical or chemical property, such as those in the field of immunoassays. The particular label used is not critical to the present invention, so long as it does not interfere with hybridization of the probe or primer. However, probes directly labeled with fluorescent labels (e.g. fluorescein, Texas red, etc.) are preferred for chromosomal DNA hybridization. In a preferred embodiment, the label is detectible in as low copy number as possible to maximize the sensitivity of the assay and yet be detectible above any background signal. The label preferably has a highly localized signal. Thus, particularly preferred fluorescent labels include fluorescein-12-dUTP and Texas Red-5-dUTP.

The present invention also includes the nucleotide sequences described herein, and their complements, which are useful as hybridization probes or primers for an amplification method, such as polymerase chain reaction (PCR), to show the presence or absence of one or more markers of the present invention. Probes and primers can have all or a portion of the nucleotide sequence (nucleic acid sequence) of the probes and primers specifically exemplified herein or all or a portion of their complements. For example, sequences shown in FIGS. 5A-5F, 6A-6K and 7A-7P can be used.

In addition, the invention pertains to isolated nucleic acid molecules consisting of a nucleic acid sequence selected from the group consisting of SEQ ID NOS: 1-20, 61-108, 205-273 and 412.

This invention also provides diagnostic kits for the detection of chromosomal abnormalities or alterations on the Y chromosome. In a preferred embodiment, a kit includes one or more probes for the markers of the invention. The kits can additionally include blocking nucleic acid (i.e., Cot-1 DNA) and instructional materials describing when and how to use the kit contents. The kits can also include one or more of the following: various labels or labeling agents to facilitate the detection of the probes, reagents for the hybridization including buffers, a metaphase spread, bovine serum albumin (BSA) and other blocking agents, tRNA, SDS sampling devices including fine needles, swabs, aspirators and the like, positive and negative hybridization controls and so forth.

The invention also provides diagnostic kits for the detection of chromosomal abnormalities or alterations on the Y chromosome using amplification methods. In a preferred embodiment, a kit includes one or more primers for the markers of the invention. The kits can additionally include amplification reagents and instructional materials describing when and how to use the kit contents. The kits can also include one or more of the following: various labels or labeling agents to facilitate the detection of the primers or amplification products, and sampling devices including fine needles, swabs, aspirators and the like, positive and negative hybridization controls and so forth.

The present invention is illustrated by the following exemplification, which is not intended to be limiting in any way. The teachings of all publication referenced herein are incorporated herein by reference in their entirety.

Exemplification

There has been a report of a novel deletion of part of the azoospermia factor c (AZFc [MIM 415000]) region of the human Y chromosome (Fernandes et al. 2004). This article reported that the deletion is found only in branchNof the Y-chromosome genealogical tree, occurs through one mutational pathway, is ˜2.2 Mb in size, and has no effect on spermatogenesis. We, too, recently reported this deletion, which Fernandes et al. termed the “g1/g3” deletion and which we termed the “b2/b3” deletion (Repping et al. 2004). Our findings, however, differed from those of Fernandes et al. in several important particulars: (1) our screening of 1,563 men demonstrated that this deletion is not confined to branch N and that it has at least four independent origins; (2) our analysis revealed two mutational pathways, rather than one, that can generate the deletion, and we confirmed the existence of the inverted AZFc organizations that are the intermediate steps in these pathways; (3) on the basis of the reference sequence of the Y chromosome, we concluded that the size of the deletion is 1.8 Mb, rather than ˜2.2 Mb; (4) using interphase FISH, we confirmed the amplicon organization that was postulated in the deletion and also identified three instances of duplication subsequent to the deletion; and (5) because of the possibility of a compensatory factor on Y chromosomes in branch N and because of the limited number of deletions outside this branch, we concluded that a possible effect of this deletion on risk of spematogenic failure cannot be excluded (Repping et al. 2004).

Beyond these differences, however, the characterizations of this and other partial deletions of AZFc (Repping et al. 2003) highlight a more important question. At issue is the relative utility of sequence family variants (Saxena et al. 2000), compared with that of plus/minus STSs, for identification and differentiation of deletions involving AZFc. AZFc is composed entirely of amplicons—repeat units 115-678 kb in length that only differ by ˜1 nt per 3,000 bp. These rare differences are called “sequence family variants” (SFVs). We previously relied on SFVs to map and sequence the AZFc region of one man's Y chromosome (Kuroda-Kawaguchi et al. 2001). The report by Fernandes et al. (2004) emphasized the use of SFVs in identification of the novel deletion, whereas our analysis relied on plus/minus STSs for identification of the deletion, followed, in most instances, by confirmation with FISH.

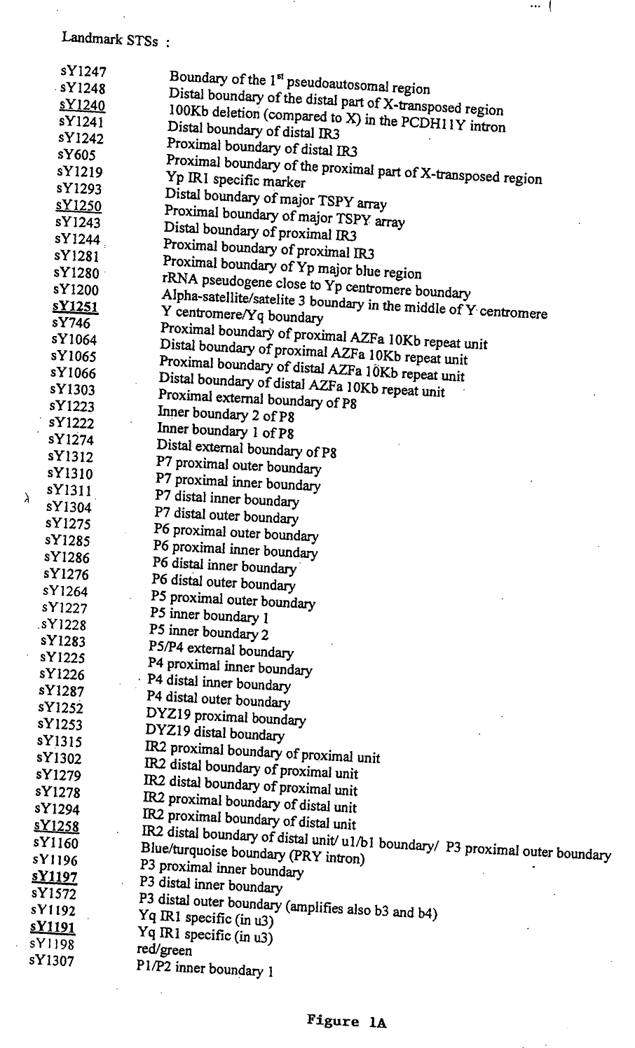

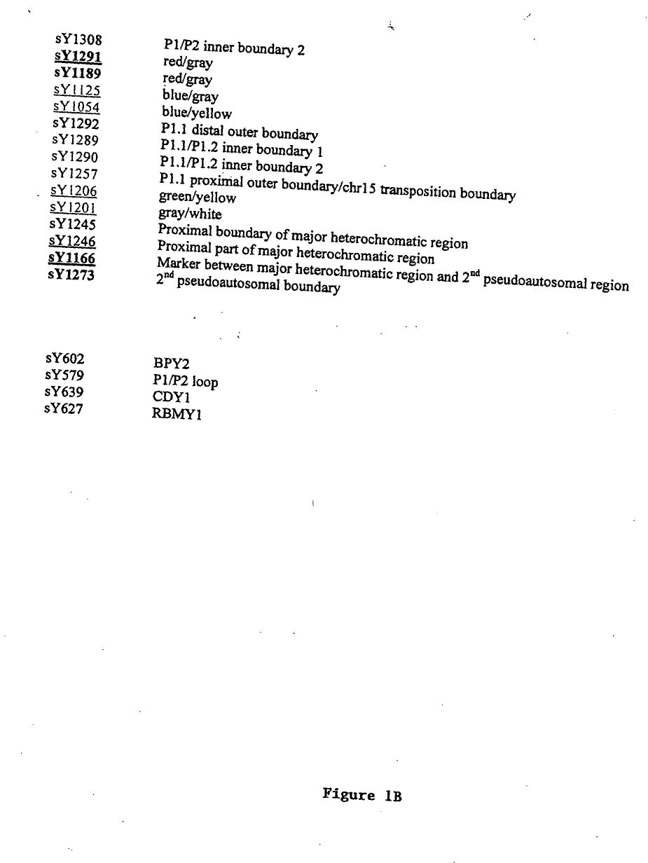

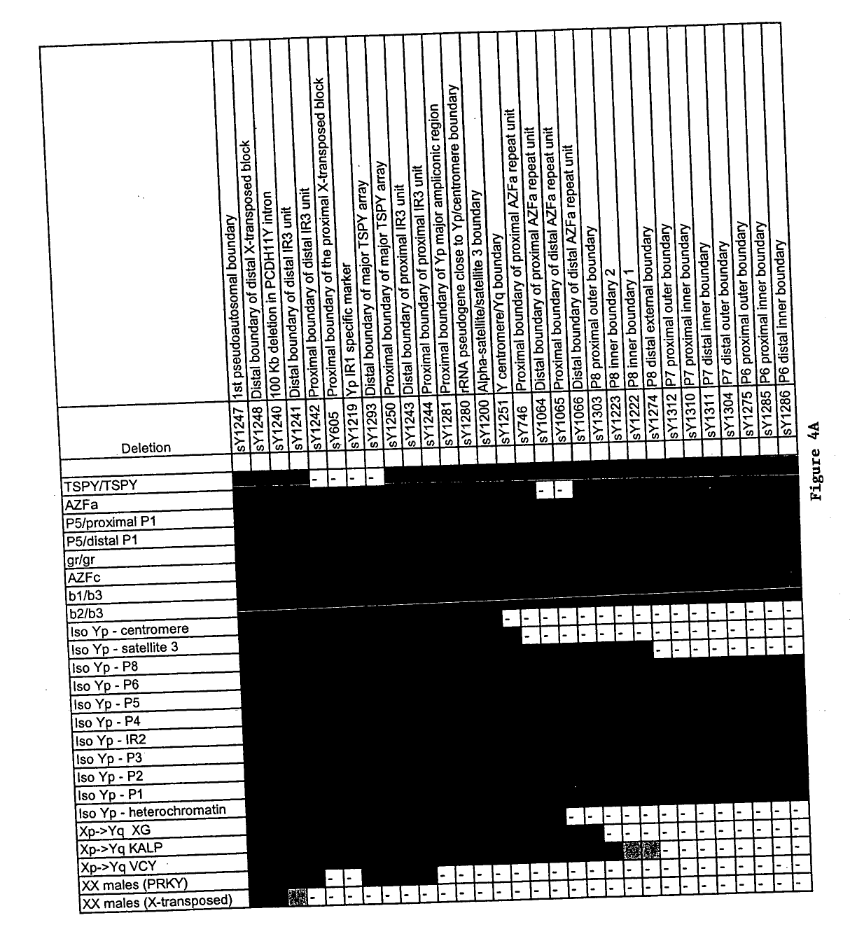

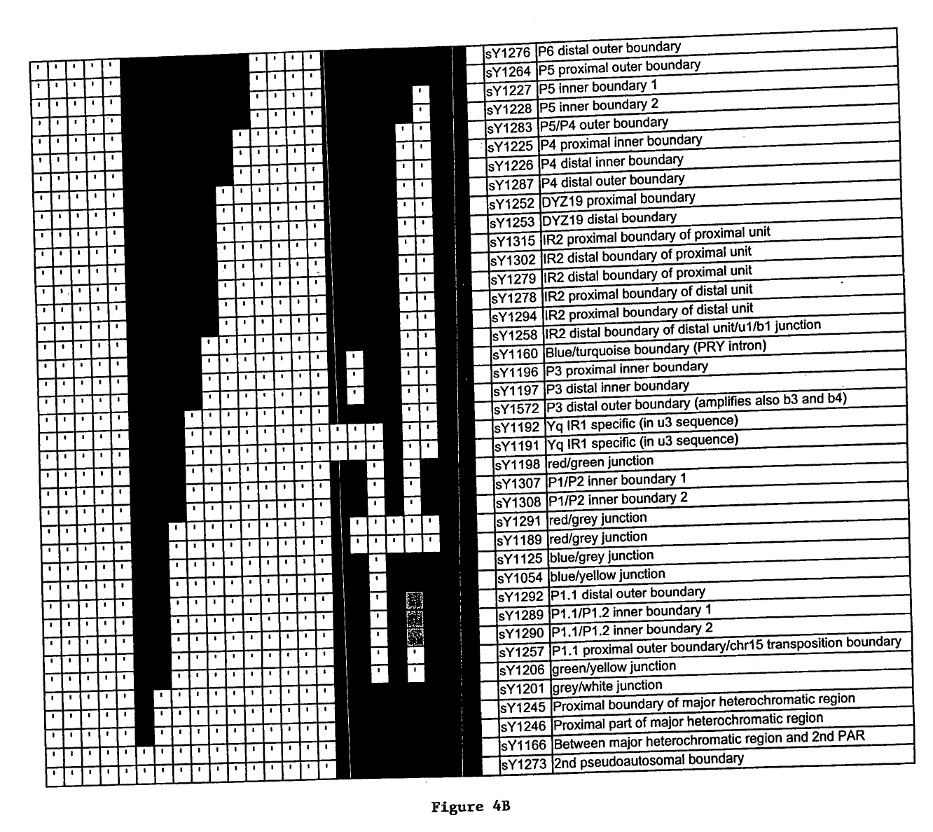

Two observations led us to ask whether SFVs, as opposed to plus/minus STSs, offer the simpler and more robust means of detecting and distinguishing deletions in AZFc (see GenBank Web site for STSs and SFV assays). First, FIGS. 1 and 4 in the report by Fernandes et al. (2004) indicated that negative results at the plus/minus STS sY1192 or 50f2/C combined with positive results at flanking STSs are sufficient to detect the deletion (table 1). Moreover, the b2/b3 deletion and other types of deletions involving AZFc can be distinguished by their plus/minus signatures, without the use of SFVs (table 1). Second, table 2 in the report by Fernandes et al. (2004) showed that the SFV patterns of undeleted chromosomes vary considerably among different branches of the Y chromosome genealogy and that the patterns also vary among individuals within branches. These observations suggested that the link between SFV patterns and particular types of deletions would likely not be consistent across the worldwide diversity of Y chromosomes.

The diversity of SFV patterns in undeleted chromosomes is not surprising, since AZFc is subject to large inversions, deletions, and duplications caused by ectopic homologous recombination between amplicons (Kuroda-Kawaguchi et al. 2001; Repping et al. 2003, 2004). Such events would rearrange the locations of particular variants and would blur the association between SFV patterns and particular types of deletions. The association would likely be further blurred by gene conversion, which frequently erases small sequence differences (i.e., SFVs) between amplicon copies on the Y chromosome (Rozen et al. 2003).

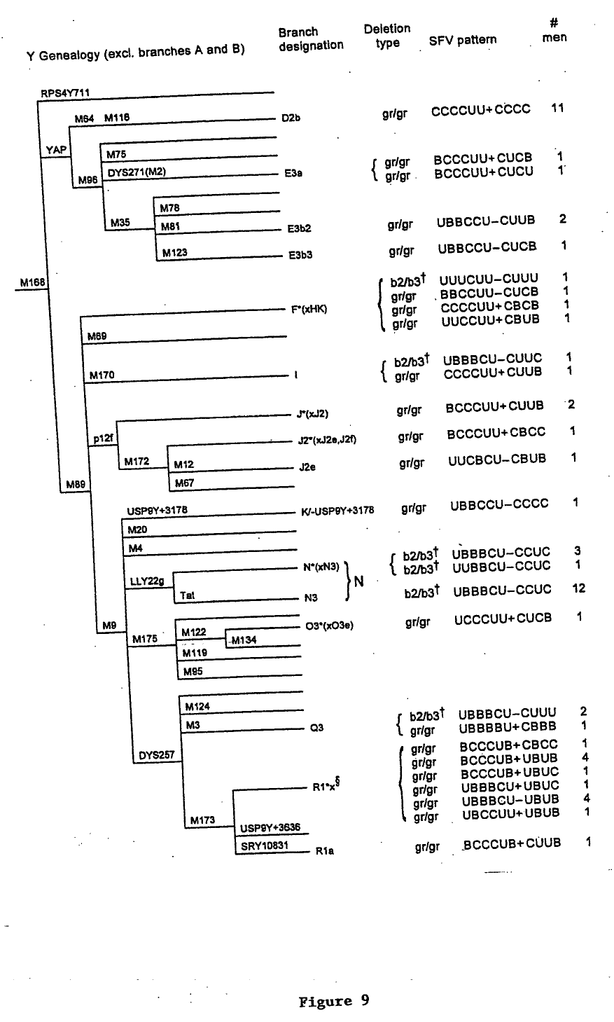

We experimentally investigated the consistency of SFV patterns in different types of deletions involving AZFc. First, using the SFVs employed by Fernandes et al. (2004), we typed 20 men reported elsewhere to have the b2/b3 deletion (Repping et al. 2004). These men represented branch N and three other branches of the Ychromosome genealogy (FIG. 9). Second, using the same SFVs, we typed 40 men reported elsewhere to have the gr/gr deletion, the other common deletion in that part of AZFc (Repping et al. 2003). These men represented 14 branches of the Y-chromosome genealogy (FIG. 9). The b2/b3 deletions outside branch N showed diverse SFV patterns, and the gr/gr deletions showed even greater diversity. This greater diversity was likely due to the larger number of independent gr/gr deletions studied. Two branches, F*(xHK) and R1*x, contained numerous deletions and a high diversity of SFV patterns. In these branches, multiple independent deletion events probably account for the high diversity. By contrast, two other branches, D2b and N, contained numerous deletions but uniform SFV patterns. This uniformity is explained by the fact that all chromosomes in these branches descended from deleted founders (Repping et al. 2003, 2004; Fernandes et al. 2004). Thus, the chromosomes in each of these branches represent single-deletion events.

Our data also showed that the SFV patterns of b2/b3 and gr/gr deletions are not distinct from each other. For example, the b2/b3 pattern UUUCUU-CUUU (branch F*[xHK]) is more similar to the gr/gr pattern UUCCUU+CBUB (branch F*[xHK], four differences [underlined]) than to the b2/b3 pattern UBBBCU-CCUC (branch N, six differences). In another example, the gr/gr pattern UBBBCU-UBUB (branch R1*x) is more similar to the b2/b3 pattern UBBBCU-CUUC (branch I, three differences) than to the gr/gr pattern BCCCUB+CBCC (branch R1*x, 10 differences).

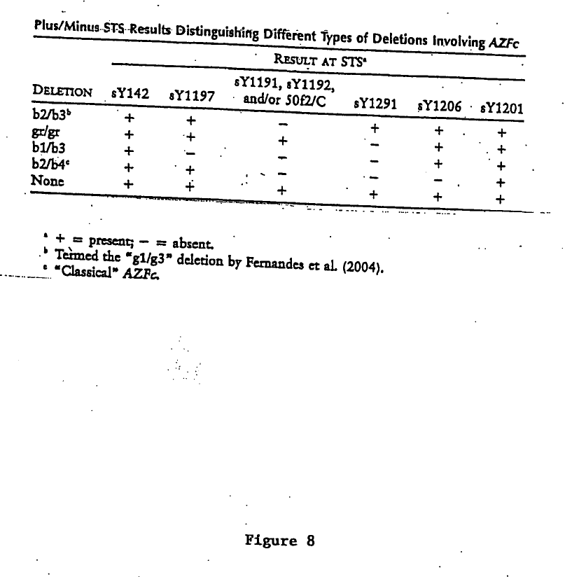

In conclusion, the SFV patterns of b2/b3 and gr/gr deletions vary widely and are not clearly distinct. SFVs can offer insight only if one knows the common SFV organizations in the genealogical branches represented by the Y chromosomes being tested. However, SFV organizations across the Y-chromosome genealogical tree are largely unknown, and SFV patterns vary even among individuals in the same branch. Just as important is that a large number of two-step assays are needed for SFV typing and for determining the Y-chromosome branch. By contrast, six simple plus/minus STSs distinguish between the deletions involving AZFc (FIG. 8). Thus, plus/minus STSs provide a straightforward means of identifying and distinguishing the deletions of part of AZFc, whereas, in most situations, SFVs do not.

### REFERENCES

Fernandes S. Huellen K, Goncalnes J, Dukal H. Zeisler J, Rajpert De Meyts E, Skakkebaek N E, Habermann B, Krause W, Sousa M, Barros A, Bogt P H (2002) High frequency of DAZ1/DAZ2 gene deletions in patients with severe oligozoospermia. Mol Hum Reprod 8:286-298
Fernandes S, Paracchini S, Meyer L H, Floridia G, Tyler-Smith C, Vogt P H (2004) A large AZFc deletion removes DAZ3/DAZ4 and nearby genes from men in Y haplogroup N. Am J Hum Genet 74:180-187
Kuroda-Kawaguchi T, Skaletsky H, Brown L G, Minx P J, Cordum H S, Waterston R H, Wilson R K, Silber S, Oates R, Rozen S, Page D C (2001) The AZFc region of the Y chromosome features massive palindromes and uniform recurrent deletions in infertile men. Nat Genet 29:279-286
Repping S, Skaletsky H, Brown L, van Daalen S K M, Korver C M, Pyntikova T, Kuroda-Kawaguchi T, des Vries J W A, Oates R D, Silber S, van der Veen F, Page D C, Rozen S (2003) Polymorphism for a 1.6-Mb deletion of the human Y chromosome persists through balance between recurrent mutation and haploid selection. Nat Genet 35:247-251.
Repping S, van Daalen S K M, Korver C M, Brown L G, Marszalek J K, Gianotten J, Oates R D, Silber S, van der Veen F, Page D C, Rozen (2004) A family of a human Y chromosomes has dispersed throughout northern Eurasia despite a 1.8 Mb deletion in the azoospermia factor c region. Genomics (advanced online publication)
Rozen S, Skaletsky H, Marszalek J D, Minx P J, Cordum H S, Waterston J H, Wilson R K, Page D C (2003) Abundant gene conversion between arms of palindromes in human and ape Y chromosomes. Nature 423:873-876
Saxena R, de Vries J W A, Repping S, Alagappan R K, Skaletsky H, Brown L G, Ma P, Chen E, Hoovers J M N, Page D C (2000) Four DAZ genes in two clusters found in the AFZc region of the human Y chromosome. Genomics 67:256-267
Skaletsky H, Kuroda-Kawaguchi T, Minx P J, Cordum H S, Hillier L, Brown L G, Repping S, et al (2003) The male specific region of the human Y chromosome is a mosaic of discrete sequence classes. Nature 423:825-837
Underhill P A, Shen P, Lin A A, Jin L, Passarino G, Yang W H, Kauffman E, Bonne-Tamir B, Bertranpetit J, Francalacci P, Ibrahim M, Jenkins T, Kidd J R, Mehdi S Q, Seielstad M T, Wells R S, Piazza A, Davis R W, Feldman M W, Cavalli-Sforza L, Oefner P J (2000) Y chromosome sequence variation and the history of human populations. Nat Gen 26:358-361 Y-Chormosome Consortium (2002) A nomenclature system for the tree of human Y-chromosome binary haplogroups. Genome Res 12:339-348

1. A method of detecting an alteration in the human Y chromosome comprising assessing a nucleic acid sample from an individual to be tested for the presence or absence of one or more nucleic acid molecules comprising a nucleic acid sequence selected from the group consisting of SEQ ID NOS: 1-20, 61-108 and 205-273, wherein the absence of one or more of said nucleic acid sequences is indicative of an alteration in the human Y chromosome in the individual.

2. A method according to claim 1, wherein the AZFc region of the Y chromosome is altered.

3. A method according to claim 1, wherein the alteration is a deletion in the Y chromosome.

4. A method according to claim 3, wherein the deletion is selected from the group consisting of the deletions shown in FIGS. 2, 3A-3B, 4A-4B and 8.

5. A method according to claim 1, wherein the nucleic acid sample is a genomic DNA sample.

6. A method according to claim 1, wherein the individual to be tested is a male with reduced sperm count.

7. A method according to claim 1, wherein the presence or absence of said one or more nucleic acid molecules is determined using one or more probes complementary to the nucleic acid sequence.

8. A method according to claim 7, wherein said one or more probes are immobilized on a solid support.

9. A method according to claim 8, wherein said one or more probes are contained in a microarray.

10. A method according to claim 1, wherein the presence or absence of said one or more nucleic acid molecules is determined by amplification using one or more primers complementary to the nucleic acid sequence.

11. A method according to claim 1, wherein the nucleic acid molecule comprises a nucleic acid sequence selected from the group consisting of SEQ ID NOS: 1-20.

12. A method according to claim 11, wherein the presence or absence of the nucleic acid molecule is determined using one or more corresponding primers selected from the group consisting of SEQ ID NOS: 21-60.

13. A method according to claim 1, wherein the nucleic acid molecule comprises a nucleic acid sequence selected from the group consisting of SEQ ID NOS: 61-108.

14. A method according to claim 13, wherein the presence or absence of the nucleic acid molecule is determined using one or more corresponding primers selected from the group consisting of SEQ ID NOS: 109-204.

15. A method according to claim 1, wherein the nucleic acid molecule comprises a nucleic acid sequence selected from the group consisting of SEQ ID NOS: 205-273.

16. A method according to claim 15, wherein the presence or absence of the nucleic acid molecule is determined using one or more corresponding primers selected from the group consisting of SEQ ID NOS: 274-411.

17. A method of predicting or aiding in the prediction of the likelihood of success of an infertility treatment of a male having reduced sperm count, comprising assessing a nucleic acid sample from said male for the presence or absence of one or more nucleic acid molecules comprising a nucleic acid sequence selected from the group consisting of SEQ ID NOS: 1-20, 61-108 and 205-273, wherein the absence of one or more of said nucleic acid sequences is indicative of an alteration in the human Y chromosome in the individual, and determining the likelihood of success of a fertility treatment in view of the type of alteration present, if any.

18-32. (canceled)

33. An isolated nucleic acid molecule comprising a nucleic acid sequence selected from the group consisting of SEQ ID NOS: 21-60, 109-204 and 274-411.

34. A kit comprising one or more isolated nucleic acid molecules comprising a nucleic acid sequence selected from the group consisting of SEQ ID NOS: 21-60, 109-204 and 274-411, amplification reagents, and instructions for using said nucleic acid molecules and reagents to detect the presence or absence of one or more nucleic acid molecules comprising a nucleic acid sequence selected from the group consisting of SEQ ID NOS: 1-20, 61-108 and 205-273.

35. A method according to claim 1, wherein the nucleic acid molecules assessed are those shown in FIG. 2.

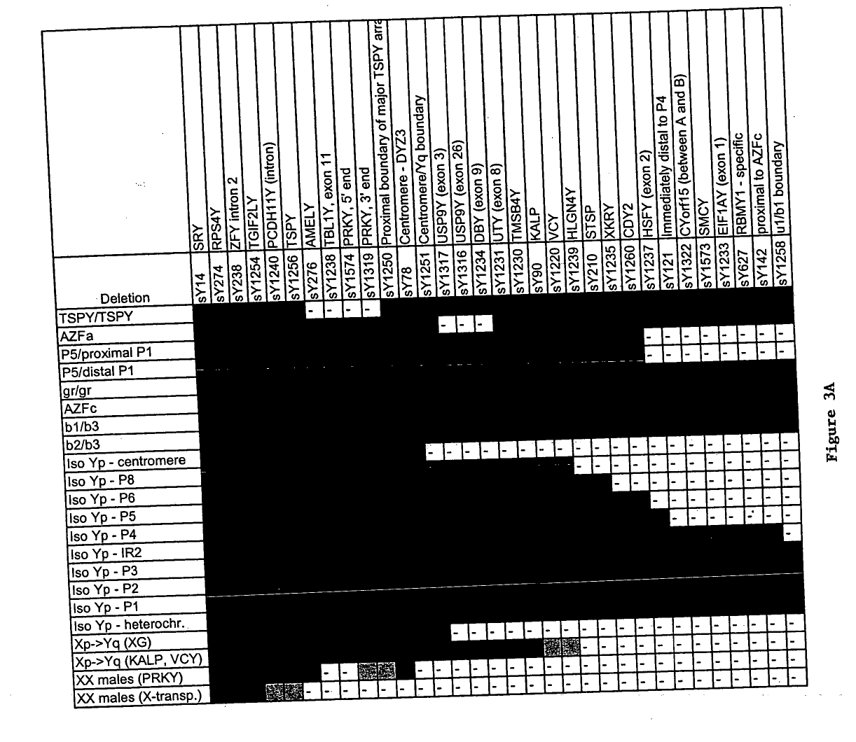

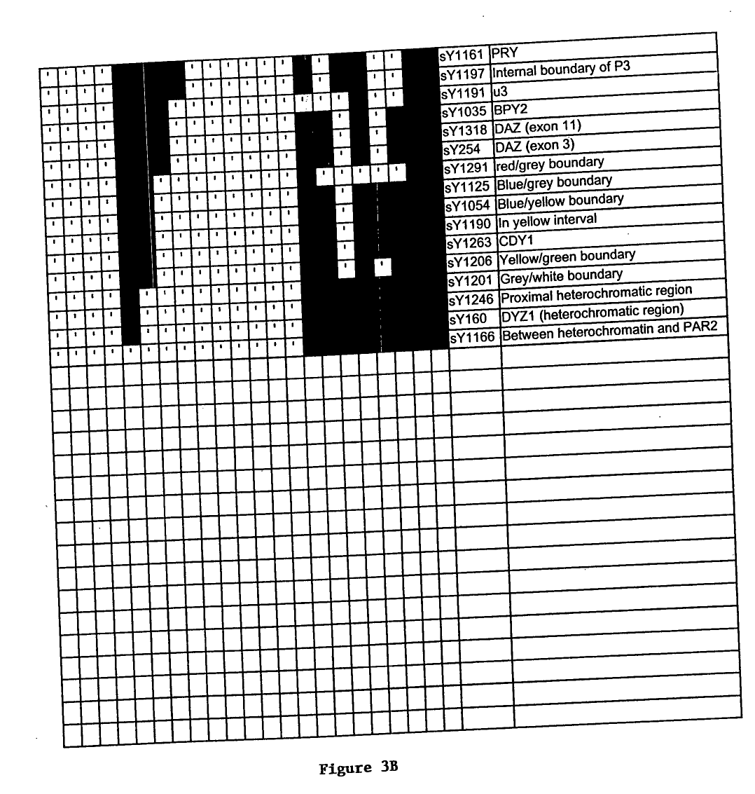

36. A method according to claim 1, wherein the nucleic acid molecules assessed are those shown in FIG. 3A-3B.

37. A method according to claim 1, wherein the nucleic acid molecules assessed are those shown in FIG. 4A-4B.

38. A method of detecting an alteration in the human Y chromosome comprising assessing a nucleic acid sample from an individual to be tested for the presence or absence of one or more nucleic acid molecules comprising a nucleic acid sequence of SEQ ID NO: 412, wherein the absence of said nucleic acid sequences is indicative of an alteration in the human Y chromosome in the individual.

39-48. (canceled)

49. A method of predicting or aiding in the prediction of the likelihood of success of an infertility treatment of a male having reduced sperm count, comprising assessing a nucleic acid sample from said male for the presence or absence of a nucleic acid molecule comprising SEQ ID NO: 412, wherein the absence of said nucleic acid sequence is indicative of an alteration in the human Y chromosome in the individual, and determining the likelihood of success of a fertility treatment in view of the type of alteration present, if any.

50-59. (canceled)

60. An isolated nucleic acid molecule comprising SEQ ID NO: 412.

61. A kit comprising one or more isolated nucleic acid molecules comprising a nucleic acid sequence selected from the group consisting of SEQ ID NOS:413-414, amplification reagents, and instructions for using said nucleic acid molecules and reagents to detect the presence or absence of a nucleic acid molecule comprising SEQ ID NO: 412.
